(19) United States
(12) Patent Application Publication
(10) Pub. No.: US 2024/0161017 A1
(43) Pub. Date: May 16, 2024

(54) Connectome Ensemble Transfer Learning

(71) Applicant: Derek Alexander Pisner, St. Petersburg, FL (US)

(72) Inventor: Derek Alexander Pisner, St. Petersburg, FL (US)

(21) Appl. No.: 18/198,262

(22) Filed: May 16, 2023

Related U.S. Application Data
(60) Provisional application No. 63/342,633, filed on May 17, 2022.

(51) Int. Cl. G06N 20/20

(52) U.S. Cl. CPC G06N 20/20

(57) ABSTRACT

The present disclosure describes a method of Connectome Ensemble Transfer Learning (CETL), which makes connectome-based predictive models useful for precision mental healthcare. CETL comprises a novel transfer learning process that incrementally trains Connectome Ensemble Predictive Models (CEPMs) by leveraging information from source domains to improve predictive performance in target domains. The disclosed methods broadly comprise selecting target and source domains, obtaining network connectivity data from individual persons, sampling source ensemble representations of connectome “views” from the obtained network connectivity data of said persons in the source domain, reducing the dimensionality of the sampled connectome “views”, and transferring the distilled representations to the target domain to train more robust, generalizable, and clinically deployable CEPMs that predict diverse target mental health phenotypes. Implemented through massively parallel distributed computing, a system of synchronized computer hardware implementing this method is also disclosed.

### TECHNICAL FIELD

The present disclosure describes a method implemented in biomedical software intended for usage by experts from a variety of fields such as connectomics, systems biology, neuroimaging, psychology, graph theory, individual differences, machine-learning, transfer learning, ensemble learning, deep learning, bioinformatics, precision psychiatry, precision medicine, digital phenotyping, computer science, cognitive science, and artificial intelligence.

### CROSS REFERENCE TO RELATED APPLICATIONS

The present disclosure claims priority to the provisional application U.S. 63/342,633, filed May 17, 2022.

### 1 Ensemble Transfer Learning for Precision Medicine

### 1.1 What is Precision Medicine?

Broadly construed, precision medicine (alias precision healthcare) is the practice of tailoring medical treatment to the phenotypes of an individual patient. Its purpose is to improve health outcomes by making medical decisions based on the unique genetic, epigenetic, environmental, lifestyle, and demographic characteristics of each patient. Precision healthcare is an approach that uses information about a person's genes, environment, and lifestyle to arrive at more precise diagnoses, forecast disease prognoses, discover new treatments, and tailor existing treatment plans to promote optimal treatment response.

In the specialized context of precision mental healthcare (alias precision psychiatry) specifically, a primary objective is to leverage genetic, epigenetic, psychosocial and other biomarkers (e.g., brain imaging) to identify subgroups of individuals with similar neuropsychological traits, including patterns of therapeutic response. The current standard of care in psychiatry is “one-size-fits-all” treatment, which has demonstrated limited success. For example, the majority of patients with major depressive disorder do not achieve full remission with the first antidepressant they try, the consequences of which is financially and clinically devastating for most. In particular, depressed patients for whom the first round of treatment fails are, on average, likely to experience 7-8 subsequent Major Depressive Episodes (MDEs) [48], spending as much as 21% of their lifetime in a chronically depressed state [86]. Hence, depression, like most forms of mental illness, is distinct as a medical condition in terms of its unique susceptibility to the logistics of treatment selection and delivery. A central goal of precision psychiatry is therefore to match patients to personalized treatment regimens a priori, which preemptively raises the chances of treatment success before treatment delivery. In this way, precision mental healthcare may succeed where previous attempts to develop blunt “silver-bullet” cures have largely failed. It views the fight against mental illness as one that more efficiently translates to developing more intelligent processes of treatment selection and administration, as a necessary complement to more efficacious drugs, surgeries, therapeutic orientations, or neuromodulation technologies outright.

To this end, Artificial intelligence (AI) plays a pivotal role in facilitating precision medicine processes, especially in the context of mental healthcare for which it may be especially lucrative due to the complexity of mental illness itself. In particular, AI is uniquely well-suited to efficiently process and analyze vast amounts of complex and heterogeneous human subjects data in a manner that can facilitate activities like biologically-informed diagnosis, genetically-informed drug contraindication profiling, treatment matching and recommendation, and disease remission, relapse, and periodicity forecasting. Despite these promises, early attempts to apply traditional deep-learning and other machine-learning methods have proven limited due to fundamental constraints on the available data, but also due to the complexity of mental illness in and of itself. Given the scarcity of labeled data points (i.e. human patients), the heterogeneity of those patients, and the inherently high dimensionality of the labels as defined according to ever-fluctuating and often-arbitrary diagnostic criteria, the AI models developed so far in support of precision mental healthcare have tended to under-perform. More specifically, they have tended to both “underfit” (i.e. failing to precisely capture the full complexity of disease mechanisms at work) and “overfit” (i.e. failing to generalize across heterogeneous patient populations and clinical scenarios, even when the full complexity of disease mechanisms have been accounted for). Despite these setbacks, the comparative success of AI in other, more data-abundant and less mechanistically complex health domains (e.g. cancer diagnosis and prognosis) continues to serve as an inspiration of what can be achieved through precision medicine. Consequently, researchers have yet to relinquish hope that, under the right technological circumstances, such a paradigm might still be achievable in the less straightforward context of mental healthcare. In the following sections and en route to the Claimed innovation, we will first delve into an overview of two such existing methods. The first is Ensemble Transfer Learning, which combines the strengths of both ensemble and transfer learning to overcome the inherent challenges of working with limited, complex, and heterogeneous data. The second, is connectome ensemble learning (i.e. the inventor's prior innovation patented in U.S. Ser. No. 11/188,850B2), which capitalizes on the model over-specification problem encountered in traditional connectomics research, and repurposes it as a generative machine learning tool capable of probing hierarchical brain network features that are directly implicated in mental disorder maintenance and development.

### 1.2 Toward an Ensemble Transfer Learning Methodology

### 1.2.1 Defining Ensemble Learning

Ensemble learning is a machine learning technique that combines multiple models or feature representations to improve overall predictive performance, often by reducing overfitting, increasing generalization, and providing more accurate predictions [57, 71]. The broad idea behind ensemble learning is that a group of diverse learning agents can make better predictions together (i.e. as a “committee of experts”) than they can alone, because they can capture distinct albeit complementary views of data. In effect, this creates a stabilizing mechanism of self-correction that curbs the errors and biases of the individual models. There are several popular ensemble learning methods [36], including:
Bagging (Bootstrap Aggregating): Bagging involves training multiple base models, usually decision trees (or so-called “random forests” of decision trees), on different subsets of the training data sampled with replacement (i.e., bootstrapping). The final prediction is usually obtained, for instance, by averaging the predictions of the base models for regression tasks or by taking a majority vote for classification tasks. Bagging helps to reduce overfitting by introducing diversity in the training data for each base model, and it often results in a more stable and accurate ensemble model.
Boosting: Boosting is an iterative technique that adjusts the weights of training instances based on the performance of previously trained models. Initially, all instances have equal weights. In each iteration, a new model is trained on the weighted instances, and the weights of incorrectly classified instances are increased so that the new model focuses more on those challenging instances. The final prediction is obtained by a weighted combination of the base models' predictions. Boosting can improve the accuracy of weak learners by focusing on the most difficult instances in the training data.
Stacking (Stacked Generalization): Stacking involves training multiple base models on the same training data, and then using another model, called the meta-model or meta-learner, to combine the base models' predictions. The base models are trained on the original training data, and their predictions are used as input features for training the meta-model. The meta-model learns to make a final prediction based on the base models' predictions, effectively leveraging their strengths and mitigating their weaknesses.
Multi-view Representation Learning: Multi-view learning is a type of ensemble learning that focuses on exploiting multiple representations or views of the same data to improve prediction accuracy and learning efficiency. This approach assumes that different views can provide complementary information, leading to a more comprehensive understanding of the underlying patterns in the data. This technique combines elements of deep generative modeling with Multiverse Analysis (MA) [76], which involves fostering greater statistical robustness through a “many worlds” interpretation that assumes the existence of a superset of equally plausible features or models that conceivably all capture distinct information, with some measurable degree of bias, about the same underlying latent phenomenon to be learned. Multi-view learning methods can be broadly categorized into three groups:
   Co-training: Co-training is a semi-supervised learning method that trains two or more classifiers on different views of the same labeled data and uses their agreement on the unlabeled data to iteratively refine their predictions. The classifiers are assumed to be conditionally independent given the true label, which allows them to collaboratively learn from each other's confident predictions on the unlabeled data.
   Multi-view Clustering: Multi-view clustering techniques aim to improve clustering performance by integrating information from multiple views of the data. These methods typically involve either co-regularization, where multiple clustering algorithms are regularized to agree on their clustering assignments, or late fusion, where clustering results from individual views are combined to produce a final consensus clustering.
   Multi-view Feature Learning: Multi-view feature learning methods focus on learning a shared or latent representation of the data that aligns or fuses information from multiple views [88]. These techniques, which include canonical correlation analysis (CCA) and its variants, learn a joint embedding space for the different views, allowing for more effective feature extraction and data analysis.

In conclusion, ensemble learning methods offer a powerful way to improve the performance of machine learning models by combining the strengths of multiple models or feature representations, making it a valuable tool in a wide range of applications. Although a decade of research has underscored the vital role of ensemble learning for grappling with the inherent complexity of models in precision mental healthcare [19], it is at best—necessary but not sufficient for training performant AI at scale.

### 1.2.2 Defining Transfer Learning

TL is a broad principle of statistical learning with a wide variety of implementations and applications, but it generally refers to the ability of a predictive model to leverage knowledge learned from one problem instance (a source domain) to more efficiently solve a related but different problem in a target domain [93]. Specifically, given a source domain [symbol] _{S} and learning task [symbol] _{S} in that domain, as well as a target domain [symbol] _{T}, and learning task [symbol] _{T} in that domain, such that [symbol] _{S}≠ [symbol] _{T}, or [symbol] _{S}≠ [symbol] _{T}, transfer learning seeks to boost performance of a target discriminative function ƒ_{T}(·) in [symbol] _{T} using the knowledge from [symbol] _{S} and [symbol] _{S} to “pretrain” or initialize ƒ_{T}(·) [93]. As in ensemble learning, a core motivation behind TL is to foster greater predictive resilience in the face of high problem complexity without abundant training data. But in order to capture a more comprehensive understanding of limited data in a target domain, ensemble learning attempts to combine the strengths of multiple diverse learners, whereas TL effectively reuses previous learning from related domains (i.e. with abundant data) so it can adapt (i.e. “fine-tune”) its learning to the target domain in “few[er]-shot[s]”. Statistically, TL works by exploiting the joint probability of multiple, often heterogeneous, source datasets through an incremental learning process that draws on a priori knowledge and experience [93]. In this sense, TL might be most easily conceptualized in Bayesian terms it seeks to augment model performance by economically building a posterior distribution based on prior knowledge accrued across a variety of source domain(s). Rather than training a separate model for new tasks across large datasets, TL can instead reduce the amount of data needed to perform generalizable deep learning. In essence, this strategy serves to offset the need for massive datasets in a target domain by relegating the responsibility of data abundance to so-called “pre-training” in one or more source domains where collected data is already abundant and/or abundance can be simulated through complementary ensemble learning techniques employed in tandem.

TL can be implemented in a variety of contexts (e.g. computer vision, natural language processing, dialogue systems, recommender systems, activity recognition, and bioinformatics) and in a variety of ways, which broadly fall into one of two loose categories: model-based transfer and feature-based transfer. Model-based TL tends to include instance-based TL, relation-based TL, heterogeneous TL, homogeneous TL, adversarial TL, multi-task TL, transitive TL, AutoTL, privacy preserving and federated TL, deep TL, Bayesian TL, knowledge representation TL, ensemble TL, adaptive TL on graph neural networks, and reinforcement TL). Feature-based TL tends to include semi-supervised TL, discriminability-based TL, Bayesian TL, correlation-based TL, manifold-based TL, subspace-based TL, and multi-view TL. Although now widely used in the field of machine learning, TL originated in educational and learning psychology research tracing back more than a century ago [29, 91]. As in its later machine learning application, transfer of learning in the context of psychology refers to the process of influencing future learning and performance in a target environment based on prior experience gleaned from an external environment [29, 91]. For this reason, TL is sometimes conceptualized in terms of knowledge reuse [39], case-based reasoning (CBR) [1], learning by analogy [17], and domain adaptation [77]. But across all of these cases, the fundamental goal of TL is to learn from experience an especially critical aspect of human intelligence that promotes efficient knowledge encoding (e.g., heuristics) and higher-level abstraction. Indeed, research has shown that these advantages are analogous to those that make TL successful when employed in the context of statistical computation [4, 84, 93].

### 1.2.3 how does Transfer Learning Assist with Predicting Phenotypes?

Since its inception, TL has been applied widely across research settings, particularly in areas of natural language processing, image classification, recommendation systems constrained by data sparsity or cold-start issues, and biomedical research in precision medicine, where limited human-subject pools often precludes the use of traditional deep learning methods [54, 60, 62, 93, 100]. In that context specifically, a variety of exciting TL implementations have recently emerged. Particularly exciting is a technique known as Depth Importance in Precision Medicine (DIPM) [18], which uses decision trees in a source domain to identify subgroups for which a preferred treatment regimen most strongly discriminates them from the overall group. In their seminal 1976 paper, Bozinovski and Fulgosi introduced the notion of applying TL to neural network models, and were the first to provide mathematical proofs for the technique. But it was Lorien Pratt's paper on discriminability-based transfer (DBT) that underscored just how much classification models would perform with TL, even beyond its original neural network formulation [61]. Pratt's idea was simple, but powerful boost learning by optimizing any classifier using a discriminative optimization objective. He achieved this using a decision tree based information gain metric that is now popularly referred to as discriminability, separability, or identifiability in the neuroimaging literature [13, 30, 31]. In essence, it expresses the proportion of between-subject (dis)similarity relative to within-subject (dis)similarity. DBT therefore transfers one of the most fundamental and phenotypically useful properties that a learning agent can have variance that optimally separates individual data into classes. Over a series of small experiments, Pratt found that when parameters of neural networks (features and models) were initialized in a source domain using a discriminability optimization objective that learning translated to substantially greater classification performance than when learning was initialized randomly. Recently, this benchmarking technique has gained traction as powerful tool for studying individual differences in precision medicine [18, 40, 58, 80, 97]. Like with ensemble learning, recent research has highlighted the great potential for transfer learning to help address the inherent complexity of models in precision mental healthcare [19], it is similarly not a cure-all solution for the performance issues plaguing AI efforts in precision mental health. One reason for this is that the success of TL relies heavily on the relevance and similarity between the source and target domains. In some cases, moreover, the transfer of knowledge may not be straightforward or even beneficial, leading to “negative transfer” or reduced performance. Another reason why TL alone is likely insufficient is that its success largely hinges on the quality and quantity of the source data, which remains fundamentally difficult to obtain from a single provider, and challenging to harmonize across multiple providers without also introducing new sources of error.

### 1.3 Ensemble Transfer Learning (ET)

### 1.3.1 how does Ensemble Learning Boost the Utility of Transfer Learning for Predicting Phenotypes?

As we have seen, both ensemble learning and transfer learning paradigms are useful for grappling with the limited, complex, and heterogeneous data typically encountered in AI research performed in precision mental healthcare. On the other hand, ensemble and transfer learning techniques attempt to overcome these obstacles differently. Broadly speaking, ensemble learning focuses on capturing diverse views of data and leveraging the strengths of multiple models or feature representations to improve overall predictive performance, while transfer learning aims to reuse and adapt knowledge gained from one or more source domains to enhance the learning efficiency and generalization capabilities in a target domain. Given their complementary nature, it is natural to consider combining ensemble learning and transfer learning to form a more robust and powerful learning framework for precision mental healthcare, wherein the target domain typically has limited labeled data, there are distribution shifts between source and target domains, and/or learning must be transferred across disparate data modalities that encode multiple dimensions of phenotypic complexity. Some of the many ways that ensemble and transfer learning can be used synergistically include:
Combining Pre-trained Models: Ensemble learning techniques like bagging, boosting, or stacking can be used to combine predictions from multiple pre-trained models obtained through transfer learning, taking advantage of their diversity to enhance generalization and robustness in the target domain.
Domain Adaptation [6]: Transfer learning can initialize an ensemble model, which is then fine-tuned to the target domain using ensemble learning techniques. This process allows the model to benefit from both shared patterns across source domains and domain-specific information in the target data.
Model Selection and Meta-Learning: Ensemble learning can be employed to select the most suitable pre-trained models for transfer learning in a given target domain using meta-learning techniques, maximizing the benefits of transfer learning by identifying the most relevant source of knowledge for a given target task.
Diversity in Model Architectures: Combining models with different architectures or machine-learning hyperparameters in the ensemble can create a diverse and robust representation of the source domain knowledge, capturing complementary information useful for transferring knowledge to the target domain.
Weighted Ensemble: Ensemble learning can assign weights to individual models based on their performance in the target domain, emphasizing more transferable models and de-emphasizing less transferable ones, leading to improved overall performance.
Stacking for Domain Adaptation: In stacking-based ensemble learning, the meta-model can be trained to recognize and adapt to the differences between the source and target domain data, bridging the gap between the domains and facilitating more effective transfer learning.
Multi-task Learning: Recent work has even advocated for the application of more radical ensemble learning strategies in support of TL, including the use of multi-view neural network models [45], to be learned in a source domain, whose latent representation layer generates predictions based on multiple, nonredundant classifiers trained in parallel or orthogonally to one another [44, 49]. In settings where multiple related learning tasks are needed simultaneously, such as those involving learning agents across multiple modalities of disease maintenance, multi-task learning can facilitate sharing information and knowledge among different tasks, improving generalization and adaptability when transferring to new tasks or domains.
Incremental Learning: Ensemble learning can facilitate incremental learning when combined with transfer learning, allowing the model to continuously learn and adapt to the evolving target domain, improving its predictive performance over time.
Robustness to Domain Shift: Ensemble learning provides a more robust solution when there is a distribution shift between the source and target domains, as the diverse set of models can capture different aspects of the data, making it more resilient to variations in the data distribution.

By combining ensemble learning and transfer learning, it becomes possible to train more robust and adaptable models for precision medicine AI that are capable of correctly predicting multidimensional phenotypes with lower generalizability error across diverse populations of individuals. In particular, the addition of ensemble learning to transfer learning helps to buffer against problem of “negative transfer”, where batch effects emerge across datasets due to global methodological or domain discrepancies. More specifically, ensemble learning can help to mitigate this by leveraging the diversity and complementary strengths of multiple models, which reduces the impact of individual model biases and limitations on overall performance. Furthermore, ensemble transfer learning can improve the stability of the learning process, making it more robust to changes in the data distribution and more capable of handling complex and heterogeneous data typically encountered in precision mental healthcare.

### 1.3.2 Multimodal Domain Adaptation

Multimodal domain adaptation is an advanced transfer learning technique that aims to leverage the information from multiple modalities to adapt models across different but related domains. This approach is particularly useful when dealing with heterogeneous data sources, such as images, text, audio, or physiological signals, which may have different feature spaces, data distributions, or diverging levels of complexity. Some popular domain adaptation methods include Maximum Mean Discrepancy (MMD) [34], Domain Adversarial Neural Networks (DANN) [72], or Adversarial Discriminative Domain Adaptation (ADDA) [79]. In general, the objective of multimodal domain adaptation is to learn a shared latent representation that captures the underlying structure and relationships across different modalities, allowing for the transfer of knowledge between source and target domains. To achieve this, multimodal domain adaptation typically involves the following steps:
Feature engineering: Extract modality-specific features from the source and target domain data using appropriate feature extraction techniques or deep learning architectures tailored for each modality.
Alignment: Align the source and target domain features by learning a shared latent representation that captures the common structure across modalities. This can be accomplished using techniques such as canonical correlation analysis (CCA), deep autoencoders, or adversarial training approaches.
Adaptation: Fine-tune the model using the aligned features and the available target domain data, incorporating modality-specific or shared information to improve the performance of the model in the target domain.
Evaluation: Assess the performance of the adapted model on the target domain data, typically using metrics relevant to the specific task, such as classification accuracy, regression error, or clustering quality.

By effectively exploiting the complementary information available across multiple data modalities, multimodal domain adaptation can enhance the transferability of models across domains, leading to improved performance and generalization in real-world applications. Domain adaptation can be particularly useful when large multimodal datasets are not available for direct model training (e.g. neuroimaging and genetic data are not available from the same patients), in which case leveraging the shared latent representations can enable effective knowledge transfer and provide more accurate and robust predictions in the target domain, even with limited or scarce data.

### 1.4 Summary

As we have seen, there is great potential in combining ensemble and transfer learning techniques for enhancing the performance of machine learning models in precision medicine. Indeed, a recent study in oncology serves as a prime example of how these techniques can be leveraged in tandem to improve the performance of drug response prediction models. By extending the classic transfer learning framework through the use of ensemble learning, the authors demonstrate the general utility of their approach using three representative prediction algorithms, including a gradient boosting model and two deep neural networks. Tested on benchmark in vitro drug screening datasets, the results show that the ensemble transfer learning framework broadly improved “out-of-sample” prediction performance across all three drug response prediction applications and with all three prediction algorithms. Still, it remains less straightforward to employ this technique in the context of precision mental healthcare specifically, where the challenges are unique due not only to the scarcity of labeled data points (i.e., human patients), but also the inherently complex, heterogeneous, and dynamic nature of the labels themselves (i.e. constantly evolving mental disorder diagnostic criteria). In this context, traditional deep learning and other machine learning methods have often under-performed, struggling to both capture the full complexity of disease mechanisms and generalize across diverse patient populations and clinical scenarios. For this reason, ensemble transfer learning alone, as applied to routine clinical records data, self-report or interview-style behavioral measures [50], and even unprocessed brain image data [3], is not fully equipped to provide a level of robustness needed to facilitate a precision mental healthcare paradigm shift. In fact, regardless of the modeling approach taken, a more robust form of feature engineering will almost certainly be unavoidable. Such features would need to be capable of reliably capturing neuropsychological phenotypes at an unprecedented level of detail. More likely than not, the best chance of achieving this would be to learn the features themselves during a pre-training phase as a feature-based transfer learning strategy. Standard behavioral data (e.g. IQ tests, mood questionnaires, genome-wide-association) are not well-suited for this, however, because they are scalar measures; they are not capable of encoding and decoding weights and biases that can be encapsulated and reused across domains. To be an entity that both learns and can be learned in the context of explaining domain-relevant variance in a mental health phenotype requires being something else altogether—another layer of models. This brings us to the notion of a connectome—a graphical model—that provides a rich and intricate representation of an individual's brain connectivity. As we will next further discuss connectomes, when themselves modeled as ensembles, are uniquely well-suited to activate promising techniques like ensemble transfer learning for precision mental healthcare.

### 2 Connectome Ensembles for Precision Medicine

### 2.1 What is a Connectome?

The term “connectome” has assumed multiple definitions since it was first coined in 2005 [35, 75]. In its original conception, it refers to the comprehensive map of the network connections that comprise an individual's nervous system, specifically the brain. In recent years, the term has been rebranded to more broadly describe network traits and states (i.e. connectivity phenotypes or “connectotypes”) which span genetic, molecular, cognitive, behavioral, and social dimensions of individual persons. Mathematically, these interrelationships can be expressed using a graphical model (alias graph, network model, adjacency matrix, connectivity matrix, CGM). A CGM comprises network connectivity data elements (NCDE), which further comprises nodes (alias vertices, parcels) and edges (alias links, connections). At a higher level, the “topology” of a CGM refers to the layout and arrangement of its nodes and edges. The topology of a CGM provides insights into the relationships between its NCDE and can help reveal important features about its latent structure, such as the emergence of communities, hubs, motifs, and other patterns of network organization that can be used to characterize the connectivity “building blocks” of an individual connectotype.

### 2.1.1 Non-Brain Connectomes

Non-brain connectomes extend the concept of connectomes to incorporate various other data modalities that contribute to our understanding of individual phenotypes. These non-brain connectomes can be categorized into behavioral, genomic, molecular, and social connectomes. Each of these connectomes captures specific aspects of an individual's phenotype, providing valuable information for precision medicine applications.

### Behavioral Connectomes

Behavioral connectomes capture the patterns of individual behavior, encompassing various aspects such as physical movement, social interactions, and physiological responses [32, 66, 92, 98]. Digital phenotyping, for example, involves the analysis of physical movement patterns, social interactions, and usage patterns of electronic devices. Additionally, the autonomic nervous system can be modeled as a network using physiological data acquired from wearable sensors, including measures of heart rate variability, skin conductance response, and body temperature.

### Social Connectomes

Social connectomes capture the structure and dynamics of an individual's social network, which can be extracted from online platforms or offline interactions. This includes demographic and socioeconomic data, social network structure, and the strength and direction of social connections. The analysis of social connectomes can reveal important insights into the impact of social factors on an individual's phenotype and their relationship with brain connectivity.

### Cognitive Connectomes

Semantic, sentiment, and symptom connectomes represent different aspects of an individual's mental and cognitive functioning, as well as their emotional and psychological states.
1. Semantic Connectomes: Semantic connectomes focus on the relationships between concepts and ideas, often represented as nodes and edges in a network. These networks can be generated through natural language processing (NLP) techniques, which analyze text information from various sources such as interviews, questionnaires, or written communication. Semantic connectomes can help reveal the underlying structure and organization of an individual's cognitive and conceptual landscape.
2. Sentiment Connectomes: Sentiment connectomes capture the emotional aspects of an individual's mental state, often derived from text or speech data. Sentiment analysis techniques can be utilized to quantify the emotional content of communication and represent it as a network of emotional connections. This can provide insights into the emotional dynamics of an individual and their relationship with brain connectivity and other phenotypic traits.
3. Symptom Connectomes: Symptom connectomes focus on the relationships between various psychological symptoms and their underlying neural correlates. These networks can be built using data from clinical assessments, self-report measures, or diagnostic interviews, capturing the complex interplay between different symptoms and their associations with brain connectivity patterns. Symptom connectomes can contribute to a better understanding of the etiology and progression of mental disorders, as well as inform personalized treatment strategies.

### Molecular Connectomes

Molecular connectomes encompass the analysis of metabolomic and proteomic profiles obtained from biological samples [70], such as blood or cerebrospinal fluid [14, 89]. These molecular profiles can provide crucial information about the biochemical processes occurring within an individual and their relationship with brain connectivity.

### Genomic Connectomes

Genomic connectomes focus on the genetic aspects of an individual's phenotype. This includes single nucleotide polymorphism (SNP) genotype data, gene expression profiles, gene regulatory networks (GRN), and DNA methylation patterns. For instance, recent work has shown that neural phenotypes can be linked to gene networks by leveraging brain-wide atlases of gene expression [63, 65]. The analysis of genomic connectomes can reveal important insights into the genetic underpinnings of various neurophenotypic traits and their relationships with spatially distributed network properties of brain connectivity, which helps bridge the gap between the transcriptome and connectome of the brain.

### 2.1.2 Brain Connectomes

Brain connectomes are unique among the multiple dimensions of network phenotypes that can be used to distinguish the traits and states of individual persons. The reason for the uniqueness of brain connectomes stems from the inherent multidimensional encoding within brain networks. Node NCDE can be defined based on multiple parcellation schemes, at multiple granularities, and based on multivariate signal properties of the underlying neural tissue. Edge NCDE can similarly be defined based on a wide variety of connectivity properties of underlying neural communication, at different scales and degrees of sparsity, and yet further still as weighted (assigned some continuous value) or unweighted (binarized/boolean), and directed or undirected. Whereas the edges of structural CGM are typically estimated based on the microstructural properties of neural white-matter, for instance, the edges of functional CGM are typically estimated based on patterns of spatiotemporal co-activation among nodes. In this context, edge are typically but not always undirected (i.e. directional information is not encoded and connections are bidirectionally invariant). Effective edges are a special case of NCDE that are directed (i.e. they encode directionality of information flow in the functional case and afferent/efferent connections in the structural case). Due to the deep multidimensional encoding of brain networks in particular, and their fundamentally direct proximity to the mental phenomena that give rise to the psychological traits and states that the present invention seeks to perturb, brain connectomes can be considered the “anchor” of Connectome Ensemble Transfer Learning (CETL) and accordingly will be covered most centrally throughout the present specification. When used to specifically model brain network dimensions of individual persons, CGM can describe structural, functional, or effective NCDE. The NCDE used to construct CGM can be measured through a variety of methods that vary depending on the relevant data modality available. Structural NCDE can be measured through histological dissection and tract-tracing techniques, but are more often inferred using neuroimaging modalities; in particular, Magnetic Resonance Imaging (MRI) and diffusion Magnetic Resonance Imaging (dMRI). Other ways of measuring structural NCDE include Magnetic Resonance Spectroscopy (MRS), Infrared Imaging (IR), Single Photon Emission Computed Tomography (SPECT), Computed Tomography (CT), and future neuroimaging modalities as of yet undiscovered. Functional NCDE is often inferred using Blood-Oxygen Level Dependent (BOLD) signal using functional Magnetic Resonance Imaging (fMRI) or functional Near-Infrared Spectroscopy (fNIRS), but also glucose metabolism using Positron Emission Tomography (PET) as well as scalp or subscalp electrical activity using Electroencephalography (EEG), Magnetoencephalography (MEG), Electrocorticography (ECoG), and single/multi-cell depth electrode recording. Along with other emerging neuroimaging modalities and future modalities that are as of yet undiscovered, functional connectomes can also be modeled based on high-density, subcortical Application-Specific Integrated Circuits (ASIC) for analog-to-digital conversion of neural spikes directly to byte-code. Though most high-resolution neuroimaging modalities are non-portable, there are also portable neuroimaging systems or mobile neuroimaging devices that can be used to obtain functional NCDE in more accessible, ambulatory settings. These portable systems include, but are not limited to, wearable EEG systems, fNIRS devices, and dry-electrode-based ECoG systems. The spatiotemporal data used to estimate functional NCDE (e.g. fMRI, EEG) are typically stored as a 4-dimensional time-series consisting of 3-dimensional image volume sets collected in series over time, whereas dMRI is also stored as a 4-dimensional dataset, but one that is fixed in time such that the 4^{th} dimension instead represents multiple directions and magnitudes of diffusion weighting sampled across a sphere (i.e. “q-space”). As such, dMRI is accompanied by a gradient table that captures the magnitude and orientation of each directional weighting (i.e. “b-values” and “b-vectors”). Effective NCDE can be inferred using various computational modeling techniques that integrate both structural and functional connectivity data. Methods for inferring effective connectivity include dynamic causal modeling (DCM), Granger causality, transfer entropy, and other data-driven or model-based approaches. These methods typically aim to reveal the underlying causal relationships and directionality of information flow between brain regions or nodes in the network.

### Brain Connectomes have Multidimensionally Encoded Node Attributes

In both functional and structural CGM, nodes can be defined using any of several parcellation methods: (1) atlas-defined (based on some a priori digital brain atlas composed of sulci and gyri cortical surface representations, or subcortical volumes, whereby each relevant brain region is assigned some index as an intensity value); (2) anatomically-defined, based anatomical parcels of grey-matter (typically represented as 3D surfaces or volumes) that have been digitally parcellated on a person-by-person basis; or (3) cluster-defined, based on spatially-distinct clusters of functional activation or structural homophily. Accordingly, nodes can be defined based on labels (represented as irregular, 3-dimensional geometrical volumes) or even more simply as spherical volumes of a radius r whose centroids correspond to x, y, z coordinates. Importantly, network neuroscientists are often interested in characterizing the properties of spatially restricted subnetworks (vis-à-vis whole-brain networks), which are sometimes referred to as Resting-State Networks (RSNs) if they were defined on the basis of their underlying patterns of functional connectivity specifically. Although nodes can be defined in several ways, they can further be ‘reduced by affinity’ (vis-à-vis selecting a subset of nodes that fall within the spatial constraints of RSNs or some manually-defined restricted network. For example, a core set of both 7 and 17 RSNs, as defined by Yeo et al. 2011 and redefined recently by Shaefer et al. 2018, have become key networks of interest for both research and clinical purposes. Ultimately, the complete umbrellas of node definition techniques falls into the category of a parcellation map, which is typically represented as a single 3-dimensional array of consecutive integer intensity values, each of which corresponds to a unique spatial assignment of nodes.

### Brain Connectomes have Multidimensionally Encoded Edge Attributes

In functional CGM, the edges are determined using a connectivity model ‘estimator’ applied to some individual X's time-series data (extracted predefined nodes using co-registered image signal from fMRI, fNIRS, EEG, MEG, or another functional neuroimaging modality). Typically, these connectivity models are based on one of two ‘families’ of statistical relation correlation and covariance. The correlation family consists of both parametric and non-parametric approaches, such as Pearson's and Spearman's rho correlation and partial correlation. Importantly, the use of these correlational approaches requires additional normalization schemes, such as a Fisher's r-to-z transformation of graph edge weights following connectivity model estimation. The covariance family consists of both traditional covariance estimation and a variety of Gaussian Graphical Models, in which the joint distribution of a set of random variables is assumed to be Gaussian and the pattern of zeros of the covariance matrix is encoded in terms of an undirected graph. The most common GGM is the inverse of the covariance matrix, also called the “precision” matrix, which is inherently sparse and thereby capable of representing only direct (as opposed to “indirect”) connections between nodes. In structural CGM, on the other hand, edges are most commonly determined by estimating the number, density, and/or integrity of white-matter fibers subtending nodes. These measures can be inferred from a tractogram, for instance, which is reconstructed using various tractographic traversal methods such as deterministic or probabilistic. This tracking process intimately depends both on the type of diffusion model fit to the data, and the method of tractography used once the model is fit. Examples of diffusion models include but are not limited to tensor, ball-and-stick, and spherical harmonic models. Whereas various methods of deterministic and probabilistic tractography also exist, the majority of them share a common set of tracking hyperparameters including step size, curvature threshold, tissue classification approach, number of samples, length threshold, and others not stated herein.

### 2.2 Connectome-Based Predictive Models (CBPM

Connectome-Based Predictive Modeling (CBPM) is an emerging research field in the domain of computational neuroscience and artificial intelligence that leverages the rich, multidimensional information embedded within connectomes to predict individual-level traits and states relevant to mental health. CBPMs aim to establish data-driven relationships between brain connectivity patterns and various cognitive, behavioral, and clinical outcomes, thereby enabling precision mental healthcare through personalized diagnosis, prognosis, and treatment planning.

### 2.2.1 What Features of Connectomes can be Used for CBPM? Deriving Connectome Feature Vectors (CFVs) from Graph Theory

One approach to CBPM involves the application of graph theory to analyze the topological properties of CGM. Graph-theoretic measures can be used to quantify the organization and efficiency of connectomes at multiple scales, encompassing global network characteristics such as modularity and small-worldness, as well as local properties like node centrality, clustering coefficient, and path length. These measures can then be utilized as features in predictive models to identify relationships between brain network topology and various mental health-related outcomes. For instance, researchers have used graph theory-based CBPMs to investigate the association between network properties and cognitive performance, psychiatric disorders (e.g., Alzheimer's disease, schizophrenia), and treatment response. While global graph metrics provide valuable insights into the network organization of CGMs, they are often less useful for CBPMs in that they can be overly reductionistic, yielded scalar values that may not capture the full complexity of the underlying connectome. In contrast, local graph metrics can provide more granular, region-specific information about the connectivity patterns of individual nodes within the network, which can be highly informative for understanding the neurobiological basis of mental health outcomes. Their chief limitation is that they can be overly specific to singular properties of a connectome's topology that may not fully capture the complex, nonlinear relationships of the multidimensional networks encountered in the human connectome.

### Deriving Connectome Feature Vectors (CFVs) from Representation Learning

Representation learning aims to automatically discover and learn meaningful features from raw data, allowing for the construction of more complex and informative representations of connectomes. In this context, representation learning involves transforming a graph into an embedding—a compact yet informative representation of its network connectivity elements (nodes, edges, or subgraphs) represented in a continuous, low-dimensional vector space that is compatible with most machine-learning models, while still preserving relevant structural, topological, or feature-based properties. Representation learning approaches offer several advantages over traditional graph-theoretic methods for CBPMs. First, they can automatically learn task-relevant features from the data without relying on a priori assumptions about specific network properties. Second, they can capture the complex, nonlinear relationships within connectomes. Lastly, they can be easily integrated with other modalities (e.g., genetic, behavioral) to form multimodal predictive models that provide a more comprehensive understanding of individual differences in mental health.

Various representation learning methods have been proposed for connectomes, including unsupervised, supervised, and semi-supervised techniques that involve spectral clustering, matrix factorization, or deep learning. Unsupervised representation learning approaches can include, for example, Laplacian Eigenmaps, Spectral Clustering, autoencoders and deep embedding algorithms (e.g. node2vec, GraphSAGE, Graph Autoencoders (GAEs), Variational Graph Autoencoders (VGAEs), Structural Deep Network Embedding (SDNE)), all of which learn a low-dimensional representation of connectomes by capturing the inherent structure and patterns within the connectivity data. The ensuing embeddings can then be consumed by traditional machine learning models (e.g., support vector machines, random forests, neural networks) to predict mental health-related outcomes. Supervised representation learning approaches, on the other hand, include things like Graph Convolutional Networks (GCNs), Graph Attention Networks (GATs), and Graph Neural Networks (GNNs), and integrate the process of feature extraction and prediction within a single end-to-end model. These approaches operate directly on the graph structure and leverage node-level information to learn a hierarchical representation of connectomes. GCNs, for example, apply convolutional filters on local graph neighborhoods, while GATs use attention mechanisms to weigh the contribution of neighboring nodes for learning node embeddings. The learned representations are then used to predict mental health-related outcomes through an appropriate output layer.

### 2.2.2 Challenges

Despite the promise of CBPMs in precision mental healthcare, several challenges remain to be addressed. One major concern is the limited availability of large-scale, high-quality datasets that are necessary to train robust and generalizable models. Collaborative efforts and data sharing initiatives are crucial to enable the development of more accurate CBPMs that can be applied across diverse populations. Another challenge pertains to the interpretability of CBPMs. While deep learning-based approaches can achieve superior predictive performance compared to traditional methods, their “black-box” nature often makes it difficult to understand the underlying relationships between brain connectivity patterns and mental health outcomes. Developing more interpretable models is essential for gaining insights into the neurobiological basis of mental disorders and for facilitating clinical adoption of these techniques. Additionally, methodological advancements are required to better account for individual differences in connectomes and to improve the personalization of CBPMs.

Perhaps one of the greatest limitations of CBPMs, though, is that connectome graphical models are fundamentally over-parameterized representations and therefore have a profound tendency to destabilize CBPMs. In particular, a major caveat of the multidimensional encoding of brain connectomes is that the added complexity comes at the cost of reproducibility. Put another way, the same brain network, modeled with different edge densities or node granularities that can confer strikingly different topological features [90]. Some of this overparameterization is a product of the calculus of graph theory itself [28], such as the classic “threshold-dependent problem” [33, 43], which was identified early in the history of connectomics research and refers to the instability of a CGMs topology as a consequence of thresholding—one of many feature hyperparameter that determines a CGMs sparsity/density of edge connections. Importantly, these feature hyperparameters should not be confused with machine-learning hyperparameters. A conservative definition of a hyperparameter commonly accepted by those skilled in the art is any parameter of a model that is set before the model estimation process begins. In the case of machine-learning hyperparameters such as regularization strength, learning rate, or the number of hidden layers in a neural network, they are set prior to the model training process and directly affect the model's performance during training and validation. Feature engineering hyperparameters, on the other hand, refer to parameters that are set during the preprocessing and feature extraction stages of the modeling pipeline, which directly affect the nature and properties of the input data used by the machine-learning models. These feature engineering hyperparameters can have a significant impact on the model's performance, as they determine the quality and representation of the input data. For this reason, in fact, most all feature engineering processes imply the use of hyperparameters, but they are not always meaningful nor is their variation always impactful. In the context of connectome based predictive modeling, feature hyperparameters constitute methodological decision junctures in the CGM feature-generating process, rather than the downstream machine-learning model that consumes those features. Since connectome features are relatively abstract data structures, usually the end result of many methodological decision junctures, they recruit an unusually wide variety hyperparameters. The hyperparameters of graphical features like CGM are also distinct in that they either directly or indirectly determine the attributes that are assigned to a CGMs nodes and edges.

As emerging studies specific to neuroimaging modalities continue to reveal, the thresholding-problem was just the “tip of the iceberg”; in the best-case scenario, there at least a dozen impactful CGM attributes that lack universal defaults [2, 5, 9, 10, 20-22, 24, 38, 51, 52, 56, 59, 73, 74, 78, 81, 82, 87, 94, 96]. Connectomics researchers have traditionally sought to address these attributes (alias feature hyperparameters) through a variety of methods, including: (1) imposing universal defaults anyway (e.g. “probabilistic tractography is always better than deterministic”) despite widespread disagreement and lack of consensus as to which defaults are “best”; (2) deferring to the defaults of connectome software or those found in existing literature [8], despite little or no justification provided for hard-coding those defaults; (3) exploiting defaults (or other hyperparameter values) willfully and non-transparently as a pernicious form of p-hacking and “over-hyping” [38]. When handled as such, the multidimensional encoding (i.e. a “multiverse”) of CGM attributes create a “perfect storm” for reproducibility failure and high test-error variance in CBPM predictions. When left uncontrolled, these attribute therefore pose a major hurdle for realistically translating connectomics into clinical theory and practice [12, 64].

In light of these challenges and limitations, future research in the field of connectomics and precision mental healthcare must focus on addressing the overparameterization problem and developing more stable and interpretable CBPMs. One potential solution lies in the development of Connectome Ensemble Learning (CEL) approaches that can effectively leverage the rich information contained within multiple connectome representations, while accounting for the variability introduced by different node and edge definitions. By integrating multiple connectome graphical models in a robust and cohesive manner, CEL has the potential to improve the predictive performance and generalizability of CBPMs, while also shedding light on the underlying neurobiological mechanisms that drive mental health outcomes. Moreover, the incorporation of non-brain connectomes, such as behavioral, genomic, molecular, and social connectomes, can provide a more comprehensive understanding of individual phenotypes and enable more personalized mental healthcare interventions.

### 2.3 Connectome Ensemble Learning

### 2.3.1 Definition

Connectome ensemble learning (CEL) is an advanced representation learning approach for connectome-based predictive modeling that addresses the limitations of traditional methods. Simply put, CEL entails systematically perturbing or varying the computational methods used to assign attributes to the nodes and edges of a CGM, which in turn generates a plurality of connectomes (i.e. an ensemble representation or “CER”) for further analysis. By subsequently applying ensemble learning techniques to the ensuing CER, CEL is able to stabilize a CBPM in the face of uncertainty resulting from a connectome's inherent complexity. In other words, by exploiting, rather than ignoring a CGMs variability, CEL transcends the limitations of traditional unitary connectome estimates in order to pave the way for more generalizable models that can be used for diagnosis, prognosis, and treatment matching in precision mental healthcare. For the remainder of this specification and subsequent claims, we will therefore refer to the fusion of ensemble learning with CBPM as Connectome Ensemble Predictive Modeling (CEPM).

Fundamentally, CEL capitalizes on the dual-role of connectomes as being both discriminative and generative representations: they are discriminative in that they represent identifiable biological and behavioral network features of individuals, that can be meaningfully compared across individuals. They are generative in that their graphical architecture allows them to learn and adapt to different problem domains. In other words, this dual-role exploits the unique properties of graphical ensembles as a tool for radical representation learning (i.e. feature engineering), while simultaneously capturing biologically real intricacies of brain networks underlying mental health phenotypes.

### Multi-View Biological Neural Networks (MV-BNNs

Multi-View Biological Neural Networks (MV-BNNs) are an embodiment of a hypergraphical model in connectome ensemble learning that leverages the complementary information available in multiple views of biological neural networks to enhance the predictive performance and generalizability of CEPMs. MV-BNNs aim to capture the diverse and complex patterns of connectivity present in brain networks across different spatiotemporal scales, data modalities, and computational representations. By integrating multiple connectome graphical models into a unified framework, MV-BNNs can overcome the limitations of traditional single-view methods and provide a more comprehensive understanding of the neurobiological mechanisms underlying mental health outcomes.

The core principle of MV-BNNs is to exploit the rich, multidimensional information embedded within connectomes by simultaneously learning from multiple views of the same data. Each view or representation of the data captures a unique aspect of the underlying biological neural network, and by combining these views, MV-BNNs can achieve better performance and generalization in CEPMs. This is accomplished by constructing an ensemble model of CER, each representing a different view of the brain network, and then using ensemble learning techniques as a fusion mechanism to combine the learned representations into a unified representation. The fusion mechanism can be implemented in various ways, such as concatenation, weighted sum, or self-attention mechanisms.

### Theoretical Basis

Through connectome ensemble learning, the overparameterization of CGM need not pose a threat to the reproducibility of CEPM; rather, it can be opportunistically exploited to curb measurement and prediction error so as to ultimately boost learning. In fact, Network Control Theory contends that the inherently non-linear connective topology of a network warrants that its multidimensional attributes be subjected to rigorous sensitivity-style analysis [23]. Performing such analyses further requires the capability to perturb CGM attributes in a controlled and systematic fashion with end-to-end reproducibility of methods. Without end-to-end reproducibility, the automatic differentiation necessary for most forms of machine-learning, including deep-learning, are neither possible nor valid. The base invention U.S. Ser. No. 11/188,850B2, currently implemented in open-source as PyNets, exposes an end-to-end connectome-generating decision-tree (‘DAG’), governed by freely-varying CGM attributes that are exposed to the user at runtime for tuning and grid/random-search. On the other hand, any methodological choices that can be reasonably hard-coded are done so to ensure total computational reproducibility of all other processes in a connectome-generating workflow. The hyperparameter grid that defines that DAG then becomes a recipe for a Connectome Ensemble Representation (CER), whose constituent CGMs refer to a common underlying network trait or state of one or more individual persons, can then be iteratively and incrementally refined by strategically selecting or discarding candidate CGM attributes based on any of a variety of feature-optimization criteria. For instance, a hyperparameter search (e.g. ‘grid search’, ‘random search’, ‘hyperband’) can be conducted using cross-validation to determine those networks that maximally contribute to explained variance (R2) in a machine-learning prediction. Conversely, error optimization criteria like MSE can be used to ‘prune’ away unpredictive CGM features at each ‘leaf’ of the DAG. These approaches should not be mistaken as being exclusive to machine-learning specifically, however. Connectome hyperparameter optimization methods can be more broadly construed as belonging to a broader class of multiverse-analytic methods. Like traditional sensitivity analysis [16, 67], MA involves studying the relative influence of modeling decisions on analytic results to foster greater statistical robustness and research transparency when “many worlds” of results are equally plausible [27, 46, 76]. Through MA, perturbing CGM attributes can enable researchers to gauge how any statistical results change as CGM attributes change. In fact, connectome ensemble learning can be implemented in the context of multiverse reliability analysis, multiverse structural equation modeling, and even Bayesian multiverse analysis, where one or more other kinds of target optimization metrics might be used (e.g. ICC, similarity, entropy, discriminability, AIC, BIC).

Perturbing a grid of CGM attributes to produce a CER has a compelling phenotypic basis in the science of real-world networks. By what the inventor has termed the Multiverse Attributed Connectome model, a connectotype is it inextricable from the attributes ascribed to its nodes and edges (either intentionally or incidentally) through the cumulative decision-tree of methods used to generate those nodes and edges, which constitute CGM attributes. This conceptualization is partially inspired by the Multiplicative Attributed Graphical model [42], which similarly argues that through their interactions, CGM attributes reveal a multiverse of topologies that a network “could” realistically take on, such that each node and edge of a network model (i.e. like a connectome) can be more flexibly represented by a probabilistic tensor of categorical attributes. This is true for social/mobile networks, genomic networks, molecular/cellular networks, semantic/knowledge networks, and especially brain networks. Brain networks, accordingly have a multidimensional encoding in time [15, 69, 78, 85, 96], space [99], and across scales [7, 25]. By this framework, single connectome estimates provide mere snapshots of dynamic, multidimensionally-encoded network neurophenotypic properties to which any computational model inherently lacks direct epistemic access.

### 2.3.2 Connectome Ensemble Feature Engineering

Feature engineering entails creating or selecting relevant features for improving a predictive model's performance. Using a variety of techniques such as extraction, feature elimination, selection, and transformation, together with the guidance of a variety of intermediate optimization criteria, meaningful features can be extracted from CERs without increasing the risk of overfitting.

### Computational Requirements

Even the estimation of unitary connectome is a challenging procedure that requires significant computational resources. Connectome ensemble learning is 3-4 orders of magnitude more computationally expensive, because it involves sampling the Cartesian product of many methodological choices used to produce a connectome. Nevertheless, connectome ensemble learning has been made possible with advances in massively parallel computing and the use of directed acyclic graphs (DAGs). Its high computational demands present unique challenges for routine implementation, but are also more formidable in that can be solved through increasingly powerful hardware beyond CPUs (e.g. GPUs, TPUs, QPUs) and grid-computing. As such, the substantial boost in performance of CEPMs make connectome ensemble learning justifies the added computational expense.

### Connectome Ensemble Feature Extraction

To initialize connectome ensemble feature engineering, one or more variants of connectome ensemble feature extraction (alias connectome ensemble sampling, connectome multiverse sampling) is needed. Connectome ensemble feature extraction is the process of generating multiple connectome representations by perturbing the computational methods used to assign attributes to the nodes and edges of a CGM. In practice, this process comprises leveraging one or more DAGs comprising one or more graph generators from the input network connectivity data of one or more individual persons, each with different CGM attributions. The output of this process is a connectome ensemble representation (CER), which consists of a plurality of CGM that capture the variability and uncertainty introduced by the different CGM attributions. In the case of brain connectomes, for instance, this might involve varying node definitions, edge definitions, connectivity models, parcellation schemes, and preprocessing steps to generate a diverse set of connectomes that capture different aspects of the underlying neural architecture.

### 2.3.3 Connectome Ensemble Representation Learning

Once a connectome ensemble has been extracted and its features have been reduced (i.e. selected or eliminated), its constituent CGM can either be represented separately or jointly. In the latter case, the CGMs can, for instance, be “matched” to one another using any one of a variety of graph matching techniques. Their corresponding network connectivity elements can also be represented in the form of a hypergraph, and their covariance can be represented using representational dissimilarity matrices. In the case of multimodal CER (e.g. comprising both structural and functional brain CGM), the constituent multimodal CGM can also be represented separately in the form of multi-layer graphs, or jointly in the form of multimodal hypergraphs or multimodal representation dissimilarity matrices.

### Connectome Ensemble Feature Selection

In determining which CGM attributes to perturb, and how much to perturb them, the user must take various considerations into account. The criteria of biological plausibility provides an ideal first conceptual filter in this regard, and amounts to a form of so-called “knowledge representation learning”. This approach is valuable because it can be employed at virtually no computational cost because it is driven by domain expertise. In one embodiment of brain connectomes, for instance, it may comprise the broad selection of one or more domain-relevant parcellations of nodes for representing one or more candidate brain networks. In another sense, it comprises the selection of CGM attributes to systematically perturb in pursuit of CER. The latter decision is driven by empirically-supported reasoning such that a candidate CGM attribute satisfies three minimal requirements: (1) it can be systematically controlled; (2) its perturbation produces measurable downstream variance in CGM topology; (3) the variance that its perturbation produces corresponds to biologically justifiable variation across dimensions of the multidimensional encoding inherent to the one or more brain networks of interest. More generally, domain knowledge can be leveraged as an initial feature-selection step through various forms, including Bayesian priors, constraints, or regularization terms in the optimization process.

Beyond domain knowledge, the user also has the option to perform the connectome ensemble feature extraction process with differing degrees of determinism vs. randomness. In the case where the user exerts greater control over the process, there are often relatively straightforward heuristics that can be used to guide that control, such as whether the software supports variation in the CGM attributes of interest, the range of variation supported for a given attribute, whether the degree of variation is computationally tractable or cost-effective, and other pragmatic optimization criteria about the intended use of the CER for downstream tasks (e.g. training CEPMs, clinical acquisition). Beyond pragmatic considerations, however, various scientific and statistical optimization criteria can be used.

Taken a step further, supervised dimensionality reduction techniques like Linear Optimal Low-rank projection, which can be used to maximize between-class variance while minimizing within-class variance relative to one or more specific intermediate optimization objectives. One example is to use domain-relevant phenotypes as the basis for auxiliary learning or multi-task learning. Such auxiliary phenotypes might include treatment mechanisms of action, etiological mechanisms of disease and recovery, and demographic characteristics, for instance. By leveraging one or more auxiliary variables that are non-redundant with the final outcome variable of interest, it is possible to ensure that select only those branches a CER which capture phenotypically meaningful information relevant to the predictive problem at hand and/or that which would be practically useful for a clinically deployable CEPM. Another example of a supervised feature-selection criterion is a domain alignment objective like Maximum Mean Discrepancy (MMD), which aims to align the distribution of features between different datasets or domains, thus enabling more generalizable representations of the underlying networks. Usually once all supervised methods have been exhausted, unsupervised methods can further be used to group similar features and subsequently select representative features from each cluster. Examples of unsupervised methods include PCA, ICA, and various forms of spectral, hierarchical, and k-means clustering. Once a small subset of candidate features of a CER have been isolated through iterative reduction and selection steps irrespective of the outcome variable of interest for a CEPM, formal hyperparameter optimization techniques such as grid-search, random-search, hyperband, gradient-based methods, or other evolutionary algorithms can then be used to learn which connectome views of CGM attribution yield the highest performing predictive models.

### Connectome Ensemble Feature Elimination

Dimensionality reduction plays a crucial role in refining the connectome ensemble representation by eliminating redundant or irrelevant features. Biological plausibility and computational feasibility, for instance, often provide a convenient and informal initial filter for reducing the dimensionality of brain CER. Using a biological plausibility criterion involves selecting node parcellations and CGM attributes based on existing scientific knowledge, while ensuring that the perturbation of these CGM attributes produces computationally feasible biologically justifiable variation in brain networks of interest. Using a computational feasibility criterion might similarly involve selecting a subset of connectome-generating methods that are currently implemented in well-tested software. Supervised methods, on the other hand, leverage labeled data to identify and retain the most informative features for the target outcome. For example, Discriminability-Based Transfer Learning can be used initially to select only those CGM attributes that most contribute to the identifiability of CGM across individual subjects. This optimization criterion is model-independent and simply serves to promote CER that are less redundant and more discriminatory in general.

Several unsupervised learning techniques are also available and offer a means to reduce the complexity of the connectome ensemble representation, ultimately leading to better fitting CEPM, while alleviating the high computational cost of connectome ensemble learning. To that end, another technique for pruning the “nodes” of a CER decision tree is to leverage reinforcement learning, where a learning agent interacts with the environment and adjusts the CGM attributes in a trial-and-error manner. The agent is rewarded for achieving better model performance based on the optimization targets, such as improved phenotypic discriminability, domain specificity, or biological plausibility. By continuously adapting and adjusting the CGM attributes based on the received rewards, the learning agent can effectively optimize the connectome ensemble learning model.

### 2.3.4 Connectome Ensemble Feature Reduction (i.e. “Embedding”

There are several algorithms that have been proposed to embed the plurality of graphs of a CER into a Euclidean feature-space that is compatible with machine-learning models. On the one hand, this can simply be performed by separately embedding each constituent CGM using the embedding methods described in prior sections. On the other hand, many of the same embedding algorithms can be modified to integrate information from multiple views of the same underlying network into a single embedded output vector. These latter embedding algorithms fall within a class of emerging techniques that we will refer to as “omnibus” embedding algorithms. In the simplest cases, for instance, Laplacian Eigenmaps and Spectral Clustering can be extended to omnibus forms—i.e. Multi-view Laplacian Eigenmaps or Co-Regularized Multi-view Spectral Clustering, respectively. Laplacian Eigenmaps can be especially valuable because they can also be extended to both hypergraphs and multigraphs, where they can be used to embed higher-order relationships across hyper-nodes/edges or parallel nodes/edges across multiple layers, respectively. Other techniques, such as Graph Neural Networks (GNNs) are particularly well-suited for a joint embedding scenario because they do not require node alignment or matching across graphs. GNNs can also be extended to both hypergraphs and multi-layer graphs, for example, in the form of Hypergraph Neural Networks or Hypergraph Attention Networks, and Multi-layer Graph Convolutional Networks or Multi-Aspect Graph embeddings, respectively.

### Multi-View Graph Neural Networks (MV-GNNs

MV-GNNs are an advanced machine learning technique designed to process and analyze graph-structured data from multiple views or modalities [47]. MV-GNNs aim to capture the complementary information available in each view and learn a unified, comprehensive representation of the underlying data. The primary idea behind MV-GNNs is that each view offers unique insights into the data, and by combining these views, the model can achieve better performance and generalization. To accomplish this, MV-GNNs typically consist of multiple graph neural network (GNN) branches, each responsible for processing a specific view. These branches independently learn representations for each view, and then a fusion mechanism combines the learned representations into a unified representation. The fusion mechanism can be implemented in various ways, such as concatenation, weighted sum, or self-attention. Self-attention, (alias “intra-attention”), is a type of attention mechanism encountered in deep learning that involves relating different positions of a single sequence in order to compute a representation of the same sequence [37]. In the context of learning MV-GNNs, self-attention provides a convenient form of multi-view fusion that efficiently and losslessly weighs the importance of different views, rather than simply summarize them or consider all of their elements equally [68, 83]. Once the multi-view representation is obtained, it can be used for various tasks like classification, clustering, or link prediction. By exploiting the complementary information in different views, MV-GNNs can enhance the performance of traditional GNNs, particularly in cases where individual views may not provide a complete understanding of the data.

### 2.3.5 Connectome Ensemble Based Predictive Modeling

Once the feature engineering process has been completed, and a refined CER has been generated, the connectome ensemble predictive modeling can begin. This stage involves employing machine learning algorithms to build models that can effectively predict the selected connectotypes of interest. The choice of machine learning algorithm depends on the specific problem and the desired outcomes. Examples of algorithms that can be used include, but are not limited to, support vector machines, random forests, neural networks, and Bayesian networks. These algorithms can be trained using supervised, unsupervised, or semi-supervised learning techniques, depending on the availability of labeled data and the desired level of interpretability of the resulting models. During the training process, the machine learning models are optimized to reduce prediction errors on the training data, which consists of a set of known connectotypes and their corresponding CER features. Various performance metrics, such as accuracy, precision, recall, and F1-score, can be used to evaluate the performance of the models and guide the optimization process. Cross-validation techniques, such as k-fold cross-validation or leave-one-out cross-validation, can be used to assess the model's generalization ability, i.e., its performance on new, unseen data. This helps to prevent overfitting and ensures that the model is robust and reliable when applied to real-world scenarios. Once the models have been trained, fine-tuned, and validated, they can be applied to new, unseen data, such as data from individual subjects outside of the training set, to predict the connectotypes of interest. The resulting predictions can be used for various applications, including diagnosis, prognosis, treatment planning, and personalized medicine.

### 2.3.6 Conclusion

Connectome ensemble learning offers a promising approach for developing more robust CEPMs in precision mental healthcare. By exploiting the multidimensional encoding of brain networks and systematically perturbing CGM attributes, this method provides a mechanism for deep generative feature engineering. This approach bridges the gap in leveraging ensemble transfer learning for constructing more accurate and generalizable AI models for precision mental healthcare. The proposed technology, as an extension and refinement of the connectome ensemble feature engineering framework initially described in U.S. Ser. No. 11/188,850B2, aims to overcome existing AI model limitations in precision mental healthcare and unlock the full potential of CEPM for a new generation of mental healthcare solutions.

### BRIEF SUMMARY

The present disclosure describes a set of computer-implemented methods of Connectome Ensemble Transfer Learning (CETL) capable of continuously improving the performance of Connectome Ensemble Predictive Models (CEPMs). CETL involves a transfer learning process that facilitates the distillation of high-dimensional Connectome Ensemble Representations (CERs) into a compressed latent form, which is conducive to more performant and pragmatic CEPM for precision mental healthcare. This novel combination of methods, implemented through massively parallel distributed computing, enables the development of more accurate, generalizable, and biologically plausible predictive models that can fill existing methodological gaps while addressing a wide range of clinical applications in precision mental healthcare.

CETL leverages experience from source domains by employing Connectome Ensemble Feature Engineering in those domains to produce source CER, and then incrementally reducing the complexity of the CER over incremental learning steps. This approach strikes a balance between exploration and exploitation, using iterative filters in ascending order of cost complexity to avoid an otherwise unmanageable multiverse of CGM-gene hyperparameter. The subsequent phase of CETL involves transferring the pre-trained CER models, usually comprising the smallest possible CER subset, from one or more source domains into one or more intermediate or final target domains for additional learning or prediction, respectively.

Another core innovations of CETL include a transfer learning process that iteratively reduces the complexity of the CER, methods of transferring CERs from high-dimensional or expensive connectome modalities like Magnetic Resonance Imaging to more economical, lower-dimensional modalities, and methods of pretraining Connectome Ensemble Transformer Models (CETMs) that can be fine-tuned for multiple research and clinical use-cases. These innovations support AI efforts in precision mental healthcare by stabilizing CEPMs, making more efficient use of limited data, enhancing the domain-specificity of connectome features, and making CEPMs more flexible for clinical deployment.

Importantly, CETL is not merely an embodiment of a machine learning with connectomes, nor is it merely an application of existing transfer learning techniques to connectome data. Rather, CETL comprises a novel set of methods that combines disparate analytic techniques from graph theory, ensemble learning, and transfer learning to effectively predict neuropsychological phenotypes of interest in target domains while leveraging knowledge from related source domains. In so doing, CETL aims to correct the generalizability failure tendency of existing methods for connectome-based predictive modeling, making CEPM both accurate, dependable, and safe for precision mental healthcare research, products, and services. Even further, CETL allows for effective integration and distillation of high-dimensional connectome data drawn from diverse data sources and modalities, thereby also facilitating the development of more pragmatic, scalable, equitable, and accessible connectome-based predictive models for precision healthcare. Similarly, CETL is not merely an embodiment of connectome ensemble feature engineering. On the one hand, CETL methods are indeed closely predicated on the abstraction of a connectome ensemble representation (CER) introduced in U.S. Ser. No. 11/188,850B2, since methods of applying machine-learning models to singular conntectome estimates have recently been subjected to a deluge of scientific criticism due to their unreliability and inability to generalize [2, 5, 9, 10, 12, 20-22, 24, 26, 33, 38, 43, 51-53, 55, 56, 59, 64, 73, 74, 78, 81, 82, 87, 90, 94, 96]. On the other hand, the present disclosure draws upon the process of engineering hierarchical CER features by reducing it to a single sampling step, within in a novel sequence of steps that leverage the engineered CER outputs to facilitate an incremental method of transfer learning, across various embodiments of source and target domains and various embodiments of person-specific network connectivity data modalities. Given that this process requires more than three orders of magnitude greater computational expense than existing methods to perform, the disclosed methods are not only non-obvious, they are also not computationally possible to achieve without a highly specialized and flexible grid computing system to support their implementation. Such a system is therefore also enclosed.

All software operations used to execute the disclosed processes and methods are conducted using one or more processing devices, where a processing device refers to a computing device or system that is capable of compiling and executing machine-readable instructions, as well as processing, analyzing, or modeling data in memory. This can include general-purpose computers or servers with Central Processing Units (CPUs) and Random Access Memory (RAM), as well as specialized hardware for graphical modeling and machine learning such as GPUs (Graphics Processing Units), TPUs (Tensor Processing Units), or Quantum Processing Units (QPUs).

At the time of this application, the software methods of CETL have been reduced to practice in the form of multiple tools, both closed-source and open-source. The open-source workflow PyNets, built with Python, SQL, CUDA, and C++, is used to perform the connectome ensemble feature engineering, and closed-source toolbox CETLearn, built with Python and R, is used to perform all other steps. CETLearn has been engineered to implement all learning steps of the CETL method, from selecting source and target domains, to fusing multiple modalities of connectome ensembles, training and evaluating the performance of CEPMs, in the target domain using the embedded CFVs. CETLearn offers built-in tools for data preprocessing and quality control, connectome ensemble feature extraction and engineering, connectome ensemble transfer learning, connectome ensemble predictive modeling, and model evaluation and performance monitoring. CETLearn is compatible with various neuroimaging data formats, supports parallel computing and can be run on a wide range of hardware configurations.

### 3 Embodiments

CETL can be applied to diverse data modalities, facilitating the integration of various data sources for a more comprehensive understanding of individual patient needs. Overall, CETL can be applied to diverse data modalities and holds high potential for advancing our understanding of brain networks and their associations with mental health, as well as for developing personalized interventions for mental health ailments. By addressing the limitations of current omic AI models and economically harnessing the power of high-dimensional connectome data, CETL paves the way for a new era of precision mental healthcare that is both scalable and adaptable.

Embodiments of the present disclosure may include a method of connectome ensemble predictive modeling, including selecting one or more target domains. In some embodiments, the selected one or more target domains may include one or more neuropsychological phenotypes of interest. Embodiments may also include obtaining one or more modalities of target network connectivity data (TNCD) from one or more pluralities of individual subjects.

Embodiments may also include selecting one or more source domains. In some embodiments, the selected one or more source domains may include one or more source phenotypes related to the one or more target neuropsychological phenotypes of interest. In some embodiments, the relationship constitutes a basis for transferring one or more connectome views between the selected source and target domains.

Embodiments may also include obtaining one or more modalities of source network connectivity data (SNCD) from one or more pluralities of individual subjects. Embodiments may also include sampling, by a processing device, one or more pluralities of source Connectome Graphical Models (sCGM) from the obtained SNCD. In some embodiments, the sampling includes one or more subprocesses of automated connectome ensemble feature engineering. In some embodiments, the one or more subprocesses initializes one or more connectome views to assign attributes to the Network Connectivity Data Elements (NCDE) of each sCGM in the one or more sampled pluralities of sCGM, wherein the sampled one or more pluralities of sCGM constitutes one or more source connectome ensemble representations (sCER).

Embodiments may also include ‘pruning’ or reducing dimensionality of the one or more sampled sCER. In some embodiments, the dimensionality reduction may include embedding, by the processing device, the one or more sCER as one or more source Connectome Feature Vectors (sCFVs). In some embodiments, the embedding projects the NCDE of the one or more pluralities of sCGM, from the one or more sCER, into one or more lower-dimensional feature-spaces. Embodiments may also include transferring the one or more connectome views of the one or more reduced sCER from the one or more source domains to the one or more target domains, the transfer further including sampling, by a processing device, one or more pluralities of target CGM (tCGM) from the obtained TNCD.

In some embodiments, the sampling may, as in the source domain, include one or more subprocesses of automated connectome ensemble feature engineering. In some embodiments, the one or more subprocesses initializes the one or more reduced connectome views from the source domain to assign attributes to the Network Connectivity Data Elements (NCDE) of each tCGM in the one or more sampled pluralities of tCGM. In some embodiments, the sampled one or more pluralities of tCGM constitutes one or more target CER (tCER). In some embodiments, the one or more source and target CER may include one or more hypergraphical or multigraphical models.

Embodiments may also include embedding, by the processing device, the one or more tCER as one or more target Connectome Feature Vectors (tCFVs). In some embodiments, the embedding projects the NCDE of the one or more pluralities of tCGM, from the one or more tCER, into one or more lower-dimensional feature-spaces. Embodiments may also include selecting one or more statistical learning models in the target domain. In some embodiments, the selected one or more statistical learning models constitutes one or more Connectome Ensemble Predictive Models (CEPMs).

Embodiments may also include training, by the processing device, the selected one or more CEPMs in the target domain using the one or more embedded tCFVs. In some embodiments, the training may include providing the one or more tCFVs of the one or more transferred tCER, for the TNCD obtained from the one or more pluralities of individual subjects as training data into the initialized one or more CEPMs. Embodiments may also include selecting one or more cost functions that measures error between the trained model's predictions and ground truth labels or data-points associated with the one or more target phenotypes. Embodiments may also include adjusting one or more parameters of the initialized one or more CEPMs based on the selected cost function while recursively partitioning the provided training data. Embodiments may also include selecting one or more testing subsets of the target phenotype data, selecting one or more independent validation datasets in the target domain, and monitoring the predictive performance of the CEPMs on the selected one or more testing subsets.

Embodiments may also include fine-tuning, by the processing device, the one or more CEPMs by configuring one or more CEPM hyperparameters. In some embodiments, the fine-tuned CEPM hyperparameters optimize predictive performance of the one or more CEPMs on the selected one or more testing subsets. Embodiments may also include evaluating, using one or more loss metrics, the predictive performance of the target neuropsychological phenotype of interest by the one or more trained, adjusted, and fine-tuned CEPMs on selected one or more independent validation datasets in the target domain. In some embodiments, the evaluation may include assessing the quality of domain adaptation and alignment.

Embodiments may also include storing, on one or more forms of non-transitory machine-readable storage media, the one or more trained outputs. In some embodiments, the stored outputs may include the weights or parameters of the one or more trained CEPM. In some embodiments, the stored outputs may include the one or more reduced connectome views of the one or more tCFVs. Embodiments may also include deploying the stored CEPM in the target domain for prediction of the one or more target neuropsychological phenotypes of interest. In some embodiments, the stored outputs may be provided to a user interface or user experience that allows the user to interact with the CEPMs, visualize the results of the evaluation, and make informed decisions about precision mental healthcare interventions based on the insights gained from the predictive performance of the CEPM.

In some embodiments, the one or more target neuropsychological phenotypes of interest may include one or more of the following: diagnostic categories of neuropsychological disease, prognostic trajectories of neuropsychological disease, or neuropsychological symptom severity; neural, cognitive, behavioral, or emotional traits or states; neuropsychological treatment outcomes, response profiles, or side-effect profiles; biomarkers of neuropsychological disease; neurodevelopmental milestones or neurodegenerative stages; genetic or environmental risk factors for neuropsychological disease; or cognitive or emotional abilities. In some embodiments, the one or more source phenotypes may include one or more of the following: neuropsychological disease or treatment mechanisms; risk or protective factors for neuropsychological disease; or genetic, epigenetic, neural, molecular, physiological, social, demographic, environmental, developmental, cognitive, emotional, or behavioral traits or states.

In some embodiments, the dimensionality reduction may include selecting one or more sCGM, or its NCDE, from the one or more sampled sCER, the selection further including isolating or identifying one or more sCGM, or its NCDE, using one or more isolation or identification criteria: biological, functional, statistical, clinical, or practical significance; explanation of variance, importance, influence, or predictability; interpretability or explainability; reliability, validity, or discriminability; covariance or mutual information; or domain expertise or prior beliefs. In some embodiments, the dimensionality reduction may include eliminating one or more sCGM, or its NCDE, from the one or more sampled sCER. Embodiments may also include pruning or removing one or more redundant, invariant, outlying, or artifactual sCGM, or its NCDE, according to one or more filtering criteria, including the following: biological, statistical, or theoretical implausibility; domain unspecificity, unpredictability, or unimportance; computational, financial, or practical infeasibility; unreliability, invalidity, or indiscriminability; bias, unfairness, or inequity; abnormality, invariance, uncertainty, sparsity, error, noise, or unrepresentativeness; or uninterpretability or unexplainability. In some embodiments, the selection or elimination may be guided by auxiliary or multi-task learning, or intermediate inferential analyses, such as Bayesian analysis, multiverse analysis, Factor Analysis, sensitivity analysis, Structural Equation Modeling, or paired model comparisons.

In some embodiments, the dimensionality reduction may include transforming one or more CGM, or its NCDE, from the one or more sampled sCER. In some embodiments, the transformation may include quantizing, normalizing, standardizing, or scaling the NCDE. Embodiments may also include fusing two or more sCGM views from the one or more sCER using one or more self-attention mechanisms, the fusion including learning a vector of attention weights for each view of the two or more sCGM views. Embodiments may also include multiplying the learned attention weights by the NCDE of the corresponding two or more sCGM views. Embodiments may also include aggregating the weighted NCDE across the two or more sCGM views. Embodiments may also include computing a fused sCER of the two or more sCGM views based on the aggregated weighted NCDE.

In some embodiments, the transferring of the one or more connectome views from the one or more source domains to the one or more target domains may include ensemble learning, the ensemble learning further including selecting one or more subsets of CGM from the one or more sCER. Embodiments may also include embedding, by the processing device, the selected one or more subsets of CGM as one or more source Connectome Feature Vectors (sCFVs), whereby the embedding projects the NCDE of the one or more pluralities of sCGM, from the one or more sCER, into one or more lower-dimensional feature-spaces. Embodiments may also include selecting one or more tree-based learning algorithms including decision trees, random forests, gradient-boosted trees, or extreme gradient-boosted trees. Embodiments may also include initializing the selected one or more tree-based learning algorithms. Embodiments may also include providing the one or more embedded sCFVs from the one or more pluralities of individual subjects as training data into the initialized one or more CEPMs. Embodiments may also include training, by the processing device, the selected one or more tree-based learning algorithms. Embodiments may also include aggregating the outputs of the trained one or more tree-based learning algorithms to generate one or more tree ensemble models. Embodiments may also include adjusting the one or more generated tree ensemble models by combining the individual predictions of the trained tree-based learning algorithms.

In some embodiments, the transferring of the one or more connectome views from the one or more source domains to the one or more target domains may include co-training with multiple CGM views in a CER, the co-training further including selecting two or more CGM views from the one or more sCER. Embodiments may also include embedding, by the processing device, the two or more selected CGM views as two or more source Connectome Feature Vectors (sCFVs). In some embodiments, the embedding projects the NCDE of the one or more pluralities of sCGM, from the one or more sCER, into one or more lower-dimensional feature-spaces. Embodiments may also include initializing one or more CEPMs in the target domain for each of the selected CGM views. Embodiments may also include providing the two or more embedded sCFVs as training data into the initialized one or more CEPMs. Embodiments may also include training, by the processing device, the initialized one or more CEPMs. Embodiments may also include iteratively updating, by the processing device, the one or more CEPMs using the predictions from the other CEPMs in the target domain. In some embodiments, each CEPM may be updated based on the predictions of the other CEPMs for the corresponding CGM view. In other embodiment, the updated predictions from the one or more CEPMs (for each of the pruned CER views) are aggregated to produce a combined prediction.

In some embodiments, the embedding of the one or more tCER as one or more target Connectome Feature Vectors (tCFVs) may include applying one or more global or local graph theory algorithms, matrix factorization algorithms, spectral clustering algorithms, manifold learning algorithms, encoders, decoders, or autoencoders to the NCDE one or more tCGMs of the one or more tCER to generate one or more corresponding tCFVs. Embodiments may also include applying one or more omnibus, multi-view, or multi-aspect graph embedding algorithms, hypergraph embedding algorithms, or multi-layer graph embedding algorithms to the complete set, a subset, or plurality of subsets of tCGM constituents of the one or more tCERs to generate one or more corresponding tCFVs.

In some embodiments, the trained one or more CEPMs may include one or more Connectome Ensemble Transformer Models (CETMs). As in the primary, non-transformer case, CETM training may include embedding, by the processing device, the one or more sCER as one or more source Connectome Feature Vectors (sCFVs). Embodiments of training the one or more CETMs may also include pretraining, by the processing device, one or more CETMs on the one or more embedded sCFVs. In some embodiments, the one or more CETMs may include one or more neural networks, and the pretraining of the CETMs may further include unsupervised, supervised, semi-supervised, self-supervised, or reinforcement learning methods. Similar to the primary, non-transformer case, the CETM embodiments may also include storing, by the processing device and on one or more forms of non-transitory machine-readable storage media, the one or more trained outputs, wherein the outputs may include the weights one or more pretrained CETM, its weights, its parameters, its corresponding one or more reduced and transferred connectome views, or its corresponding one or more embedded sCFVs. In the CETM case, embodiments include transferring the pretrained CETMs and associated model parameters from the one or more source domains to the one or more target domains. Embodiments may also include fine-tuning, by the processing device, the one or more pretrained CETMs to the target domain, whereby the fine-tuning may include a variation of training as in the primary, non-transformer case. In some embodiments of the fine-tuning, the method further includes initializing the one or more pretrained CETMs, providing the one or more embedded tCFVs from the one or more pluralities of individual subjects as training data into the initialized one or more CEPMs, selecting one or more cost functions that measure error between the model's predictions and ground truth labels or data-points associated with the selected one or more target neuropsychological phenotypes of interest. Embodiments may also include adjusting one or more parameters of the initialized one or more pretrained CETMs based on the selected cost function while recursively partitioning the provided training data.

Embodiments of the present disclosure may also include a method of transferring CERs across data modalities, the method including selecting one or more target domains including one or more target neuropsychological phenotypes of interest. Embodiments may first include obtaining source network connectivity data (SNCD) from one or more pluralities of individual subjects in one or more source domains. In some embodiments, the obtained SNCD may include a plurality of functional, structural, or effective brain Network Connectivity Data Elements (NCDE) sampled from one or more neuroimaging modalities.

Embodiments may then also include sampling, by a processing device, one or more pluralities of sCGM from the obtained SNCD. In some embodiments, the sampling may include one or more subprocesses of automated connectome ensemble feature engineering, wherein the one or more subprocesses constitute one or more respective connectome views for assigning attributes to the NCDE of each sCGM in the one or more sampled pluralities of sCGM. In some embodiments, the sampled one or more pluralities of sCGM constitutes one or more source connectome ensemble representations (sCER).

Embodiments may also include obtaining one or more proxy modalities of TNCD from one or more pluralities of individual subjects in one or more target domains. In some embodiments, the obtained TNCD may include behavioral, genomic, molecular, social, genomic, or portable neuroimaging data modalities. In some embodiments, behavioral modalities include Digital phenotypes such as physical movement patterns, social interactions, or usage patterns of electronic devices. Embodiments may also include Demographic and socioeconomic data. Embodiments may also include Speech, language, or text data obtained from interviews, questionnaires, or natural language processing analysis of written or verbal communication. In some embodiments, the obtained TNCD may include Physiological data obtained from wearable sensors, such as heart rate variability, skin conductance response, or body temperature. In some embodiments, genomic modalities may include Single nucleotide polymorphism (SNP) genotype data, Gene expression profiles, Gene Regulatory Networks (GRN), or DNA methylation patterns. In some embodiments, the obtained TNCD may include molecular modalities, including Metabolomic or proteomic profiles obtained from blood, cerebrospinal fluid, or other biological samples. In some embodiments, the obtained TNCD may include social NCDE, including Social network structure and dynamics data sampled from online platforms or offline interactions. In some embodiments, portable neuroimaging modalities include Electroencephalography (EEG), Magnetoencephalography (MEG), Functional Near-Infrared Spectroscopy (fNIRS), Transcranial Magnetic Stimulation (TMS), or Application-Specific Integrated Circuits (ASIC).

Embodiments may also include sampling, by a processing device, one or more pluralities of tCGM from the obtained TNCD. In some embodiments, the sampling may include one or more subprocesses of automated connectome ensemble feature engineering, wherein the one or more subprocesses constitute one or more respective connectome views for assigning attributes to the NCDE of each tCGM in the one or more sampled pluralities of tCGM. In some embodiments, the sampled one or more pluralities of tCGM constitutes one or more target connectome ensemble representations (tCER). Embodiments may also include aligning, by the processing device, the sampled sCER with the sampled tCER. In some embodiments, the alignment may include selecting, eliminating, or transforming one or more sCGM, or its NCDE, of the one or more sCER or tCER, based on one or more alignment optimization objectives. In some embodiments, one or more optimization objectives for the alignment may include minimization of domain discrepancy, information content, biological or functional significance, statistical significance, contribution to explained variance, compatibility with the one or more statistical learning models, or relevance to the one or more target neuropsychological phenotypes.

Embodiments may also include jointly embedding, by the processing device, the aligned sCER and tCER. In some embodiments, the joint embedding produces one or more multimodal embeddings of network connectivity (mCFV). In some embodiments, the joint embedding may include applying one or more multimodal graph representation learning algorithms. In some embodiments, the applied one or more algorithms may include multi-view graph embedding algorithms, hypergraph embedding algorithms, multi-layer graph embedding algorithms, multi-aspect graph embedding algorithms, distance algorithms, multi-modal graph embedding algorithms. In some embodiments, the one or more jointly embedded mCFVs may include one or more multimodal graph neural networks, multimodal multi-task models, Domain Adversarial Neural Networks, Canonical Correlation Models, or representational similarity models. In some embodiments, the user initializes, by the processing device, the one or more stored training outputs to opportunistically predict the one or more target neuropsychological phenotypes of interest using one or more partial mCFVs including only TNCD.

Embodiments may also include selecting one or more multimodal statistical learning models from the one or more target domains. In some embodiments, the selected one or more multimodal statistical learning models constitutes one or more multimodal Connectome Ensemble Predictive Models (mCEPMs). Embodiments may also include training, by the processing device, the selected one or more mCEPMs in the target domain using the one or more jointly embedded mCFVs. In some embodiments, the training may include providing the one or more mCFVs of the one or more aligned sCER and tCER, for the TNCD obtained from the one or more pluralities of individual subjects into the initialized one or more mCEPMs. Embodiments may also include selecting one or more cost functions that measure error between the model's predictions and ground truth labels or data-points associated with the one or more target phenotypes. Embodiments may also include adjusting one or more parameters of the initialized one or more mCEPMs based on the selected cost function while recursively partitioning the provided training data. Embodiments may also include selecting one or more testing subsets of the target phenotype data. Embodiments may also include selecting one or more independent validation datasets in the target domain. Embodiments may also include monitoring the predictive performance of the mCEPMs on the selected one or more testing subsets. Embodiments may also include fine-tuning, by the processing device, the one or more mCEPMs by configuring one or more mCEPM hyperparameters. In some embodiments, the fine-tuned hyperparameters optimize predictive performance of the one or more mCEPMs on the selected one or more testing subsets. Embodiments may also include evaluating, using one or more loss metrics, the predictive performance of the target neuropsychological phenotype of interest by the one or more trained, adjusted, and fine-tuned mCEPMs on selected one or more independent validation datasets in the target domain. Embodiments may also include storing, by the processing device and on one or more forms of non-transitory machine-readable storage media, the one or more trained outputs. In some embodiments, the outputs may include the one or more trained mCEPM, its weights, or its parameters. In some embodiments, the outputs may include the one or more connectome views of the one or more mCFVs. Embodiments may also include deploying the stored CEPM in the target domain for prediction of the one or more target neuropsychological phenotypes of interest.

the transferring of CERs can comprise transferring CERs across one or more pairs of individual persons, where the tCER constitutes the CER of the other individual person in the pair. The alignment and joint embedding of the sCER and tCER constitute the alignment of phenotypes across the pairs of individual persons. The method further includes predicting the one or more target neuropsychological phenotypes of interest in the target domain using the multimodal connectome ensemble transfer learning method with the aligned phenotypes across the pairs of individual persons.

In other embodiments, the transferring of CERs can comprise transferring CERs across one or more pairs that include an individual person and an artificially intelligent agent, where the tCER constitutes the CER of the artificially intelligent agent. Additionally, the transferring of CERs can comprise transferring CERs across one or more pairs that include an individual person and an artificially intelligent agent, where the tCER constitutes the CER of the individual person. The alignment and joint embedding of the sCER and tCER constitute the alignment of phenotypes across the pairs. The method further includes aligning one or more neuropsychological phenotypes from the individual person with the artificially intelligent agent in the pairs using the multimodal connectome ensemble transfer learning method.

Embodiments of the present disclosure may also include a system of synchronized computer hardware that implements CETL, the system including one or more processing devices for executing the various embodiments of CETL such as automated connectome ensemble feature engineering, CER pruning and any intermediate analyses in support of the pruning, CER embedding, multimodal CER fusion, domain adaptation and alignment, training CETL machine learning models, and deploying the trained models for clinical use. In some embodiments, the one or more processing devices may be configured using a distributed or parallel computing architecture, facilitating ‘batch-mode’—efficient processing of large-scale, high-dimensional, or multi-modal network data. In some embodiments, the processing devices initialize and execute one or more Directed Acyclic Graphs in support of sampling the CER.

Embodiments may also include one or more non-transitory machine-readable storage media for storing the obtained target and source network connectivity data, sampled CERs, CFVs, mCFVs as well as CEPMs, CETLs, and mCEPM, including their intermediate training data, training logs, weights and biases, parameters and hyperparameters, and any other supportive data structures.

Embodiments may also include one or more communication interfaces for facilitating the transfer of data and information between the one or more processing devices and the storage media. In some embodiments, the one or more communication interfaces may include one or more gateways configured to receive and process data from a wide variety of neuroimaging modalities, data formats, and hardware configurations.

Embodiments may also include one or more input devices for enabling users to interact with the system and provide input for selecting target and source domains, obtaining and preprocessing network connectivity data, and configuring the statistical learning models. Embodiments may also include one or more output devices for displaying the results of the evaluations, visualizations, and predictions generated by the CEPMs, enabling users to make informed decisions about precision mental healthcare interventions.

Embodiments may also include one or more user interfaces for providing a user-friendly environment to access and interact with the system, input data, configure settings, and view the results. Embodiments may also include an operating system configured to manage the system resources, execute the processes, and coordinate the activities of the one or more processing devices, communication interfaces, input devices, output devices, and user interfaces. In some embodiments, the one or more operating systems may be configured to adapt its processes and workflows dynamically based on available computational resources, data quality, and user-defined constraints or requirements.

Embodiments may also include in an embodiment, the system may include software applications or tools that enable users to monitor the performance of the CEPMs, visualize the results, and generate reports or summaries of the evaluations and predictions for use in clinical decision-making or research purposes. Embodiments may also include in an embodiment, the system may include security and privacy mechanisms to protect the confidentiality, integrity, and availability of the data, models, and results, including encryption, access controls, and data privacy-preserving techniques.

In some embodiments, the one or more communication interfaces are connected to or integrated with one or one or more electronic health record (EHR) systems, clinical decision support systems (CDSS), or health information exchange (HIE) platforms for seamless data exchange, interoperability, and real-time deployment of the CEPMs in clinical settings. In some embodiments, the system of synchronized computer hardware may include one or more cloud-based services that provide access to the connectome ensemble transfer learning methods and CEPMs through web-based interfaces, APIs, or other remote access methods. In some embodiments, the one or more output devices may be configured to provide real-time, personalized predictions and recommendations for precision mental healthcare interventions based on the CEPMs, supporting the delivery of tailored treatments and strategies for individual patients or populations.

### BRIEF DESCRIPTION OF THE DRAWINGS

FIG. 1A is a flowchart of active steps illustrating a method, according to a first main embodiment of the CETL method.

FIG. 1B is a flowchart of active steps extending from FIG. 1A and further illustrating the method, according to some embodiments of the present disclosure.

FIG. 1C is a flowchart of active steps extending from FIG. 1B and further illustrating the method from FIG. 1A, according to some embodiments of the present disclosure.

FIG. 1D is a flowchart of active steps extending from FIG. 1C and further illustrating the method from FIG. 1A, according to some embodiments of the present disclosure.

FIG. 2 is a flowchart of active steps further illustrating the method from FIG. 1A, according to some embodiments of the present disclosure.

FIG. 3 is a flowchart of active steps further illustrating the method from FIG. 1A, according to some embodiments of the present disclosure.

FIG. 4 is a flowchart of active steps further illustrating the method from FIG. 1A, according to some embodiments of the present disclosure.

FIG. 5A is a flowchart of active steps further illustrating the method from FIG. 1A, according to some embodiments of the present disclosure.

FIG. 5B is a flowchart of active steps extending from FIG. 5A and further illustrating the method, according to some embodiments of the present disclosure.

FIG. 6 is a flowchart of active steps further illustrating the method from FIG. 1A, according to some embodiments of the present disclosure.

FIG. 7A is a flowchart of active steps further illustrating the method from FIG. 1A, according to some embodiments of the present disclosure.

FIG. 7B is a flowchart of active steps extending from FIG. 7A and further illustrating the method, according to some embodiments of the present disclosure.

FIG. 8 is a flowchart of active steps further illustrating the method from FIG. 1A, according to some embodiments of the present disclosure.

FIG. 9 is a flowchart of active steps further illustrating the method from FIG. 1A, according to some embodiments of the present disclosure.

FIG. 10A is a flowchart of active steps illustrating a method, according second main embodiment of the CETL method.

FIG. 10B is a flowchart of active steps extending from FIG. 10A and further illustrating the method, according to some embodiments of the present disclosure.

FIG. 10C is a flowchart of active steps extending from FIG. 10B and further illustrating the method from FIG. 10A, according to some embodiments of the present disclosure.

FIG. 10D is a flowchart of active steps extending from FIG. 10C and further illustrating the method from FIG. 10A, according to some embodiments of the present disclosure.

FIG. 10E is a flowchart of active steps extending from FIG. 10D and further illustrating the method from FIG. 10A, according to some embodiments of the present disclosure.

FIG. 11 is a flowchart of optional active steps further illustrating the method from FIG. 10A, according to some embodiments of the present disclosure.

FIG. 12 is a flowchart of optional active steps further illustrating the method from FIG. 10A, according to some embodiments of the present disclosure.

FIG. 13 is a flowchart of optional active steps further illustrating the method from FIG. 10A, according to some embodiments of the present disclosure.

FIG. 14B-FIG. 14J are the panels of a flow diagram of embodiments, as organized according to the layout map in FIG. 14A. The flow diagram includes active steps, optional steps, and transitions according to the first main embodiment of the CETL method.

FIG. 15B-FIG. 15G are the panels of a flow diagram of embodiments, as organized according to the layout map in FIG. 15A. The flow diagram includes active steps, optional steps, and transitions according to the second main embodiment of the CETL method.

FIG. 16 is a block diagram illustrating a system that supports the first and second main embodiments of the CETL method.

FIG. 17 is a block diagram further illustrating the system from FIG. 11, according to some embodiments of the present disclosure.

FIG. 18 is a block diagram further illustrating the system from FIG. 11, according to some embodiments of the present disclosure.

FIG. 19 is a system diagram of some embodiments of the present disclosure, illustrating the components and processes involved in implementing connectome ensemble transfer learning, including processing devices, storage media, communication interfaces, input and output devices, user interfaces, and their interactions to enable feature engineering, pruning, embedding, domain adaptation, and training of Connectome Ensemble Predictive Models (CEPMs) for predicting neuropsychological phenotypes of interest. In the diagram, the system is implemented using the semantics of Amazon Web Services (AWS)—one of several possible cloud-hosting services that might be used to deploy the system. The system components include AWS Step Functions, AWS Glue, AWS SageMaker, EC2 instances, GPU, CPU, QPU, Cloud TPU, AWS SQL Database and Neptune Graph Databases, Athena, FSx for Lustre, AWS Batch, Docker and Kubernetes, Amazon API Gateway, Identity and Access Management (IAM), AWS KMS, and AWS Managed Workflows (e.g. for Apache Airflow DAG's). The diagram also illustrates the various workflows and subprocesses involved in connectome ensemble transfer learning, such as (m)CER pruning, (m)CER alignment, (m)CER embedding, (m)CEPM training, and (m)CEPM validation. Users can access the system through web-based interfaces or SSH remote terminal sessions and the system provides visualization and monitoring capabilities for the performance of the trained CEPMs. The system diagram also depicts ingoing and outgoing data streams. Ingested data can include anything from Health Information Exchange data, to private or public research data repositories or open datasets, PACS, or even directly acquired data from patients' mobile devices. Outgested data can include predictions, recommendations, outcomes, and visualizations generated by the CEPMs, which can be used for clinical decision-making, research purposes, or patient engagement through user-friendly interfaces, electronic health record systems, clinical decision support systems, or health information exchange platforms. The system architecture is designed to be adaptive, scalable, and secure, ensuring efficient processing of unimodal or multimodal network connectivity data while preserving data privacy and ensuring interoperability with various data formats and hardware configurations.

FIG. 20 is a violin plot that, according to brain-based embodiments of CETL in the present disclosure, depicts distributions of predictive R2 (i.e. the black jitter within each violin) that represent the association between each behavioral phenotype of interest (shown in the adjacent legend) and connectome features as estimated in the source domain.

FIG. 21 is a mosaic that, according to brain-based embodiments of CETL in the present disclosure, depicts stacked bar plots of discriminability decision-sensitivity. The decision-sensitivity is here faceted across columns by ‘network definition’ (also dropped from the x-axis) with functional connectomes on the top row and structural connectomes on the bottom row. In each plot in the mosaic, the x-axis contains perturbed connectome-generating attributes of interest, and the y-axis is the relative variable importance (VIP) index of discriminability sensitivity, according to Classification and Regression Trees (CART), ANOVA (F-Statistic), Kolmogorov-Smirnov (KS), along with first-order, interaction, and total indices as estimated by the Fourier Amplitude Sensitivity Test. FIG. 16 illustrates the effectiveness of the proposed connectome ensemble transfer learning method in identifying and selecting discriminative CGM attributes for various target neuropsychological phenotypes, as mentioned in the claims. The figure highlights the potential of the method to enhance the predictive performance of Connectome Ensemble Predictive Models (CEPMs) across different network definitions and connectome-generating attributes, ultimately demonstrating its applicability and adaptability in addressing a wide range of neuropsychological phenotypes and scenarios.

FIG. 22 is a bar plot depicting an embodiment of auxiliary learning informing the dimensionality reduction step of CETL, according to brain-based embodiments of CETL in the present disclosure. The height of each bar reflects the extent to which perturbation of select connectome attributes of interest in the source domain (x-axis) modulated predictions of auxiliary phenotypes (rumination and depression severity and inertia), where the neuropsychological phenotype of interest in the target domain was chronic depression. The y-axis depicts the relative variable importance (VIP) index of R2 sensitivity as defined by a Classification and Regression Trees (CART) model. FIG. 17 demonstrates the potential of using auxiliary learning to inform the dimensionality reduction step in the connectome ensemble transfer learning method, as described in the claims. By showing the impact of perturbing selected connectome attributes in the source domain on the predictions of auxiliary phenotypes, the figure highlights the importance of considering the relationships between the target neuropsychological phenotypes and auxiliary phenotypes when selecting, eliminating, or transforming CGM attributes. This approach contributes to enhancing the predictive performance and generalizability of the resulting CEPMs in the target domain.

FIG. 23 is a cobweb plot that, according to brain-based embodiments of CETL in the present disclosure, depicts quartiles of predictive R2, of each of five source data phenotypes, as ribbons that span a multiverse of connectome attributes perturbed in the source domain via a multi-task learning embodiment of the dimensionality reduction step.

FIG. 24 is a star-plot that depicts a multiverse analysis of prediction confidence interval widths (y-axis) derived from the orthogonal R2/MSE benchmarks associated with distinct structural (right) and functional (left) connectome feature-spaces as a function of their known discriminability-error estimates. Here, each point corresponds to an associated feature extraction recipe of CGM attribution, where the shape of said point corresponds to the temporal dimension of that phenotype (circle=persistence, triangle=severity).

FIG. 25 is a diagram that, according to brain-based embodiments of CETL in the present disclosure, depicts a Structural Equation Model used for intermediate analyses of CER in a source domain.

FIG. 26 is a figure mosaic that, according to brain-based embodiments of CETL in the present disclosure, illustrates both the promise of incorporating Bayesian approaches in the CETL method. In particular, Bayesian paired t-tests with Bayes factor and Leave-One-Out (LOO) log predictive density are used with N=100 posterior predictive draws of ROC AUC from Bayesian logistic regression classifiers of chronic depression and depression conversion. The figure demonstrates the potential of leveraging alternative benchmarks of model evaluation and the benefits of leveraging connectome ensembles to transfer feature-encoded priors of a key etiological mechanism of depression maintenance en route to classifying those at risk of chronic depression in the target domain.

FIG. 27 illustrates the process of fusing a plurality of graphical model views from an ensemble of connectome graphical models using self-attention mechanisms. The figure shows the learning of a vector of attention weights for each view of the plurality of sCGM views, and the multiplication of the learned attention weights by the network connectivity data elements of each of the corresponding plurality of sCGM views. Additionally, the figure demonstrates the encoding of weighted network connectivity data elements across the plurality of views and the computation of a multi-head attention fused ensemble of connectome graphical models from the plurality of views based on the encoded weighted network connectivity data elements.

FIG. 28 depicts the process of pretraining and fine-tuning one or more connectome ensemble transformer models (CETMs) using multiple source domains and a target domain. The figure illustrates the embedding of sCGM views from pruned sCER, the pretraining of CETMs using multi-view neural network architectures and unsupervised, semi-supervised, self-supervised, or reinforcement learning algorithms, and the fine-tuning of pretrained CETMs to the target domain using supervised learning algorithms. The resulting fine-tuned CETMs, referred to as Connectome Ensemble Predictive Models (CEPMs), are then stored and deployed for use in the target domain. This process demonstrates the adaptability and effectiveness of the CETL method in addressing various neuropsychological phenotypes and scenarios.

FIG. 29 is a diagram that, according to brain-based embodiments of CETL in the present disclosure, illustrates initial connectome ensemble sampling steps of multimodal connectome ensemble domain adaptation.

FIG. 30 is a diagram that, according to brain-based embodiments of CETL in the present disclosure, demonstrates an embodiment of multimodal distance-based transfer of a functional CER of an exemplar Attention Control Network (ACN), as modeled using an fMRI-based source modality (BOLD) to a target EEG-based modality (ERP). The figure illustrates the process of transferring the functional connectome model from the fMRI-based source domain to the EEG-based target domain using a straightforward representational similarity analysis, ultimately enabling the prediction of a depression phenotype in the target domain based on jointly learned representations that persist in the portable EEG modality, even when the fMRI modality has been removed.

FIG. 31 is a diagram that, according to passive behavioral-based embodiments of CETL in the present disclosure, illustrates initial connectome ensemble sampling steps of multimodal connectome ensemble domain adaptation.

FIG. 32 is a diagram that, according to active behavioral-based embodiments of CETL in the present disclosure, illustrates initial connectome ensemble sampling steps of multimodal connectome ensemble domain adaptation.

FIG. 33 is a diagram that, according to genetic-based embodiments of CETL in the present disclosure, illustrates initial connectome ensemble sampling steps of multimodal connectome ensemble domain adaptation.

FIG. 34 is a flow diagram illustrating an embodiment of the Connectome Ensemble Transfer Learning (CETL) method for multimodal domain adaptation, according to various embodiments of CETL in the present disclosure. The flow diagram begins from four exemplar modalities (brain, passive behavioral, active behavioral, and genomic), each with their own corresponding multi-view connectome ensemble representations (CER), per subject, stemming from the multidimensional attributes available from each modality. The diagram then proceeds to jointly embed the CGMs of each CER for each subject into a latent feature space. In a cross-validated fashion, the flow then seek to learn a best-fitting shared representation that selects the CGM (or sets of CGM) from each modality that best minimizes domain discrepancy (and other transfer-learning evaluation measures). Importantly, the method used to accomplish this might ideally accommodate for missing data and enable the possibility of removing a source modality (e.g., brain/neuroimaging) but still using its previously transfer-learned representations by proxy. The method then proceeds to train CEPMs in the target domain using the jointly learned representations and evaluate their predictive performance on independent validation datasets. The flow diagram demonstrates the overall structure and flow of the CETL method for multimodal domain adaptation, highlighting its potential for developing more accurate, generalizable, and biologically plausible predictive models for a wide range of clinical applications in precision mental healthcare.

### DETAILED DESCRIPTION

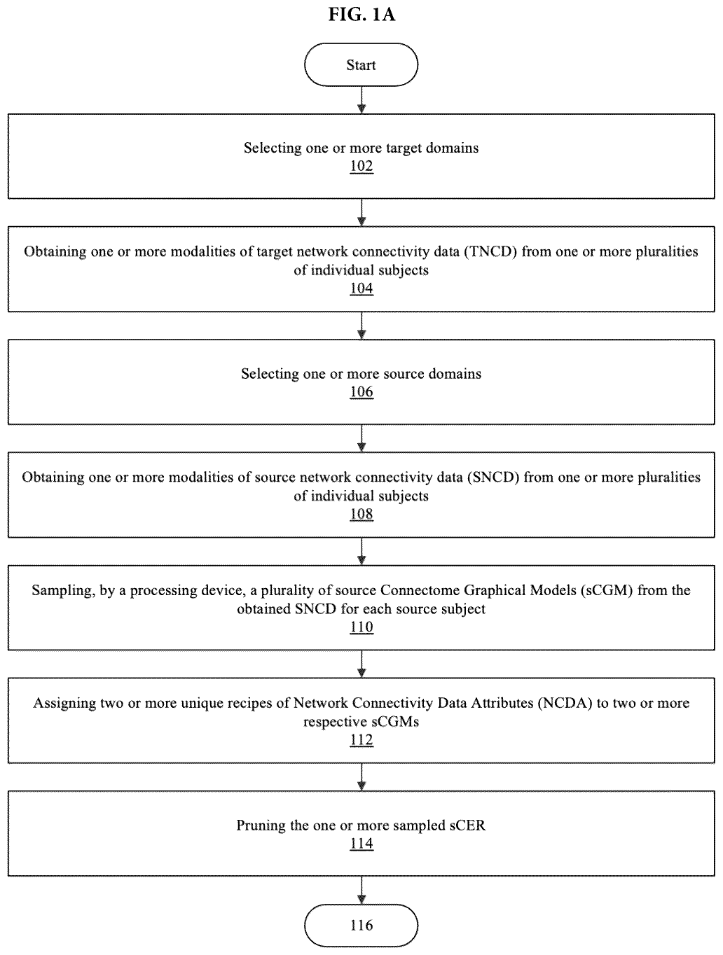

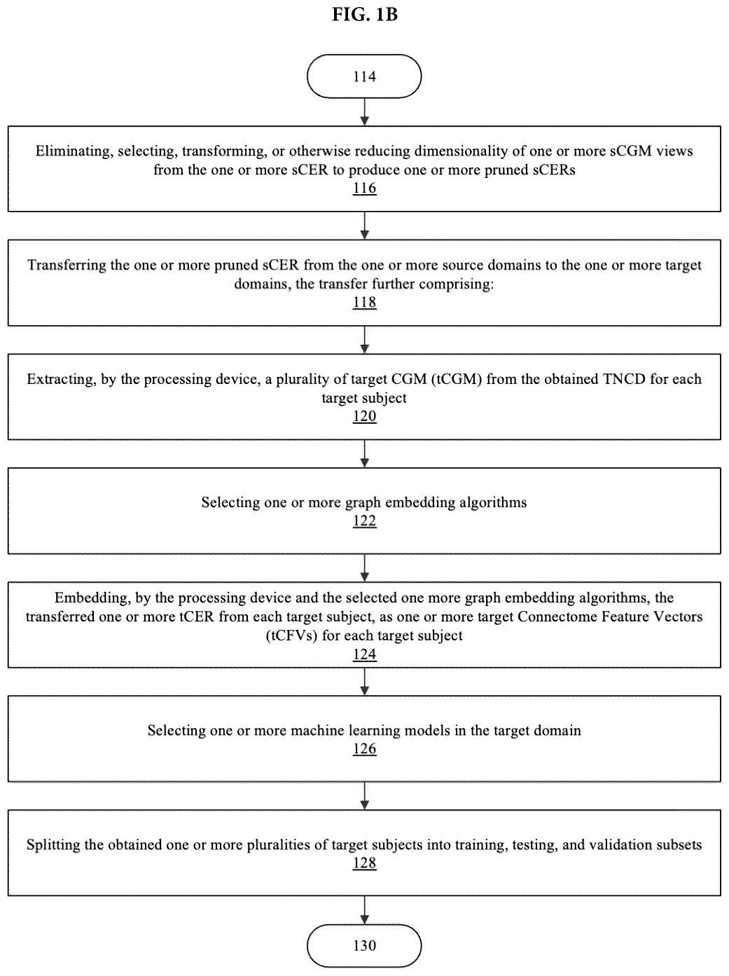

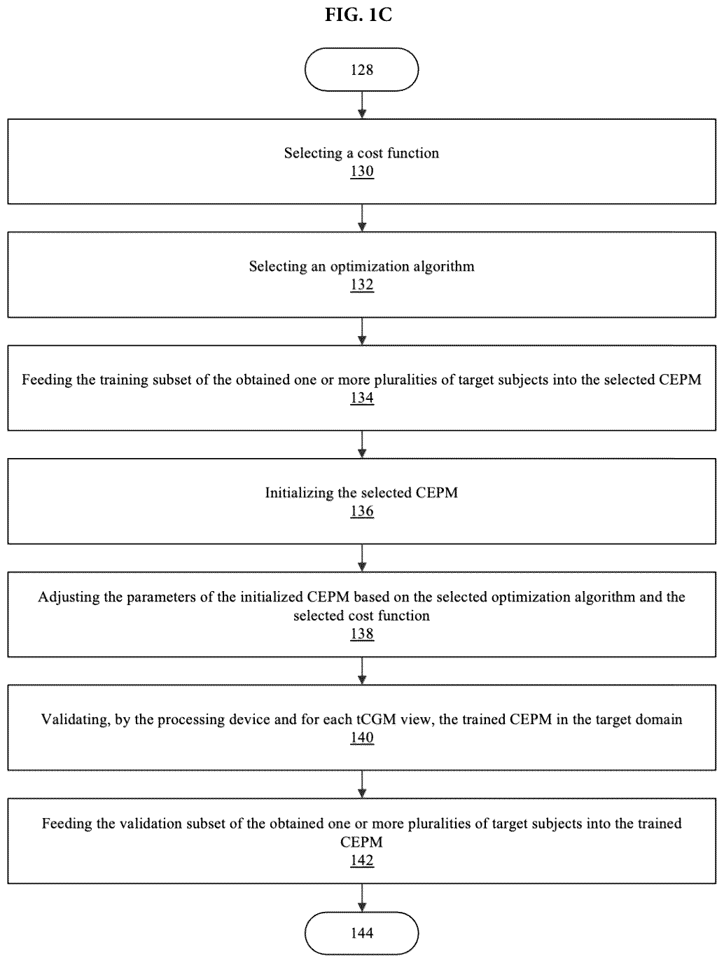

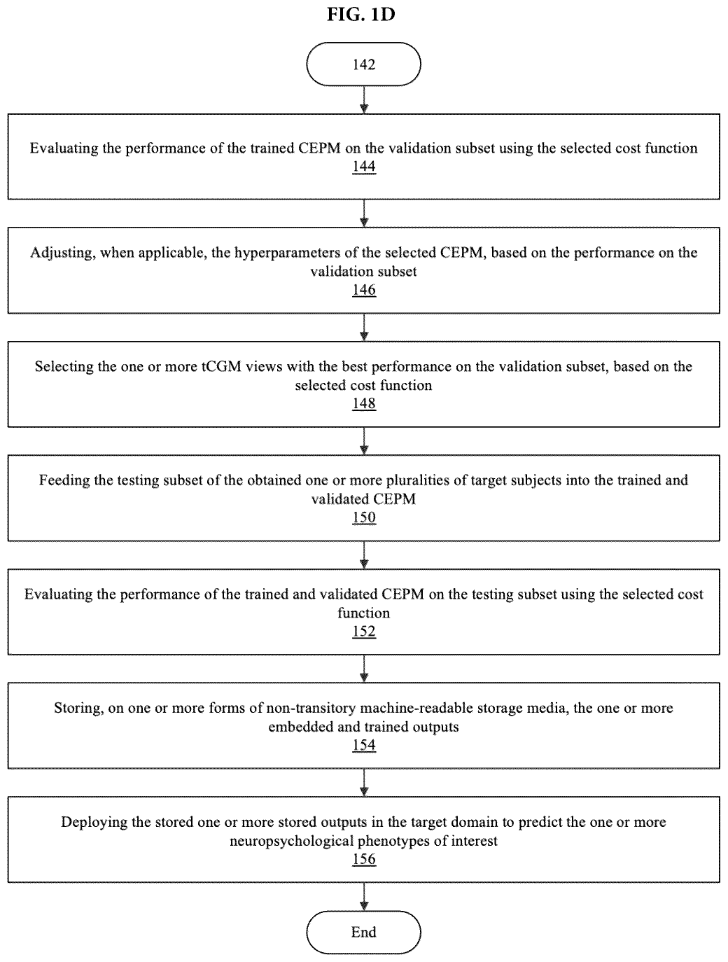

FIG. 1A to FIG. 1D are flowcharts that describe a method, according to some embodiments of the present disclosure. The method involves a series of steps that enable the prediction of neuropsychological phenotypes of interest in the target domain using Connectome Ensemble Transfer Learning (CETL).

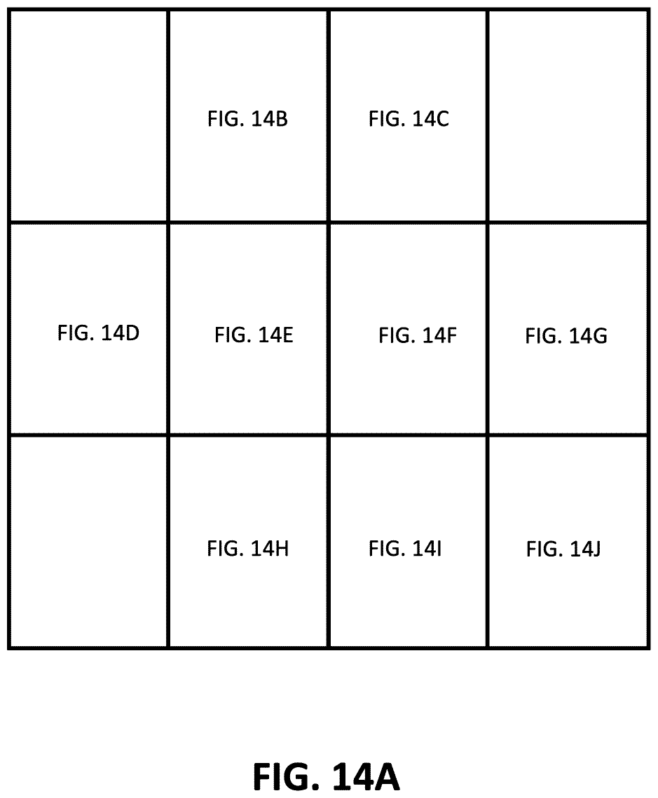

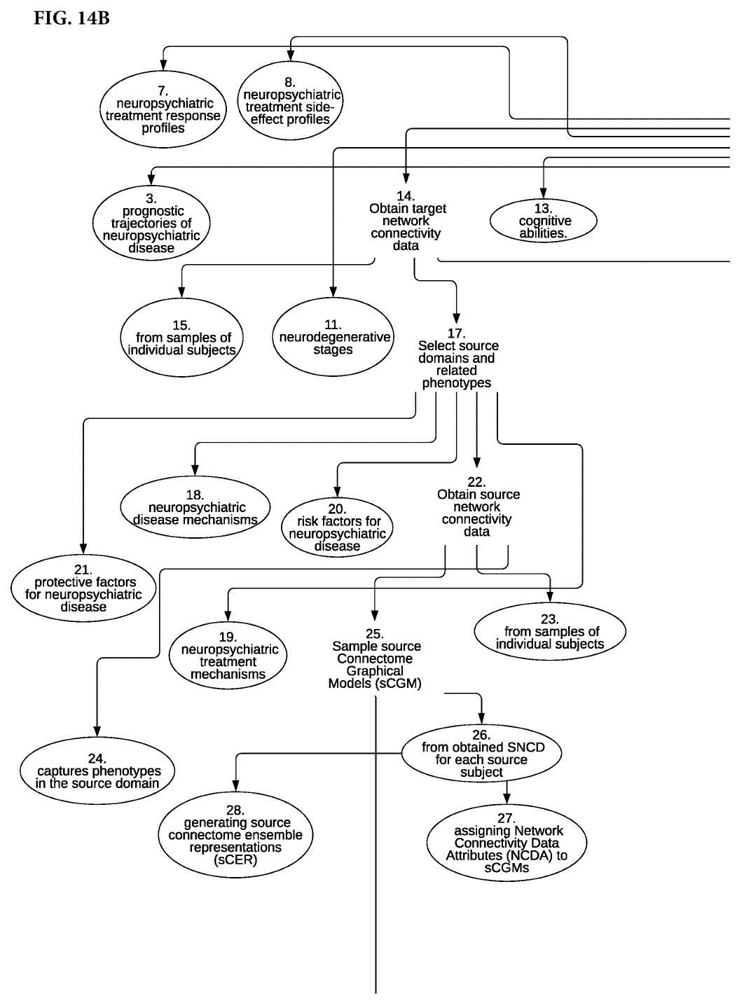

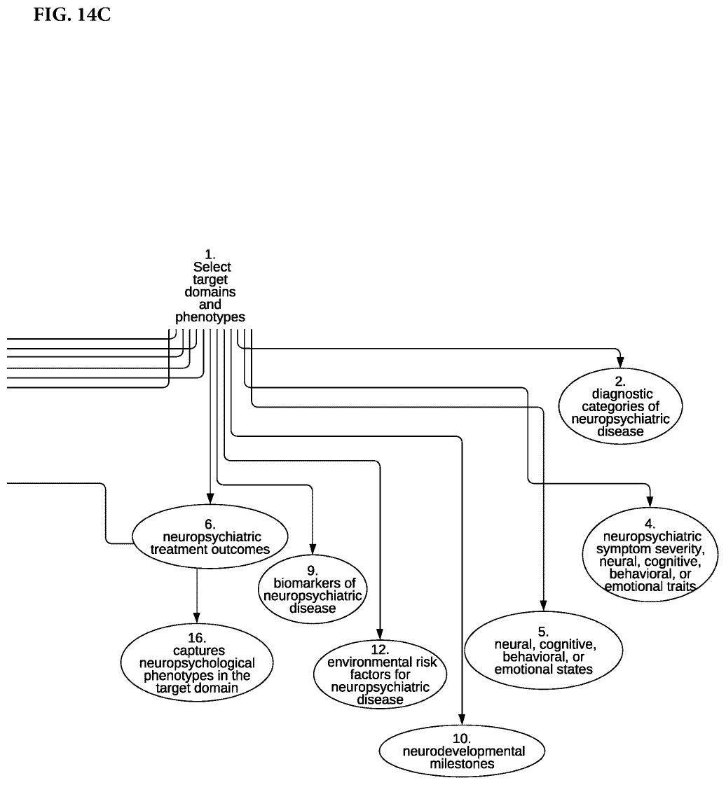

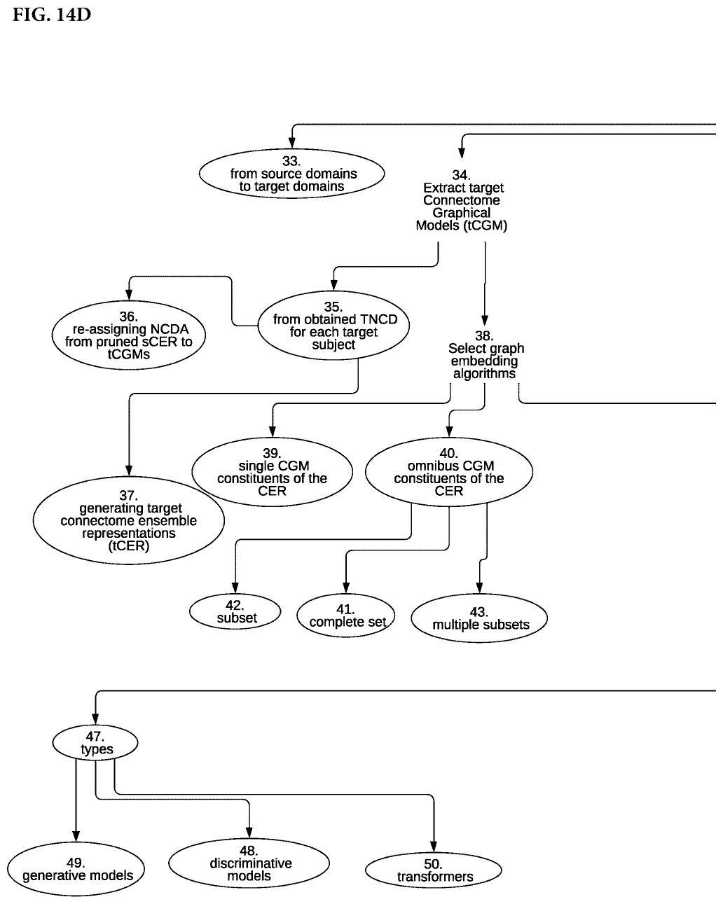

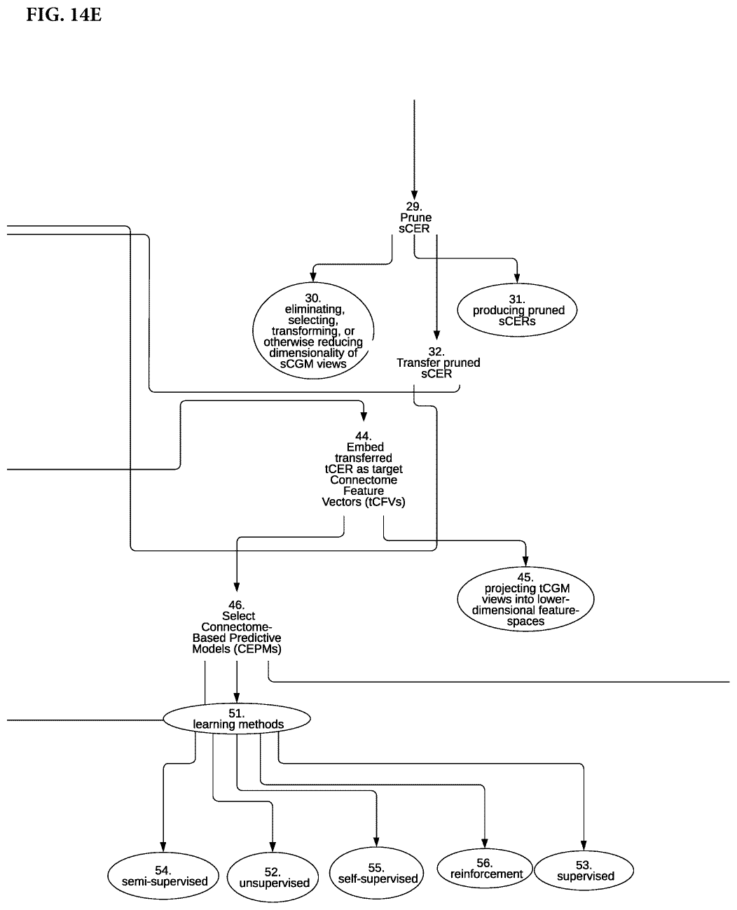

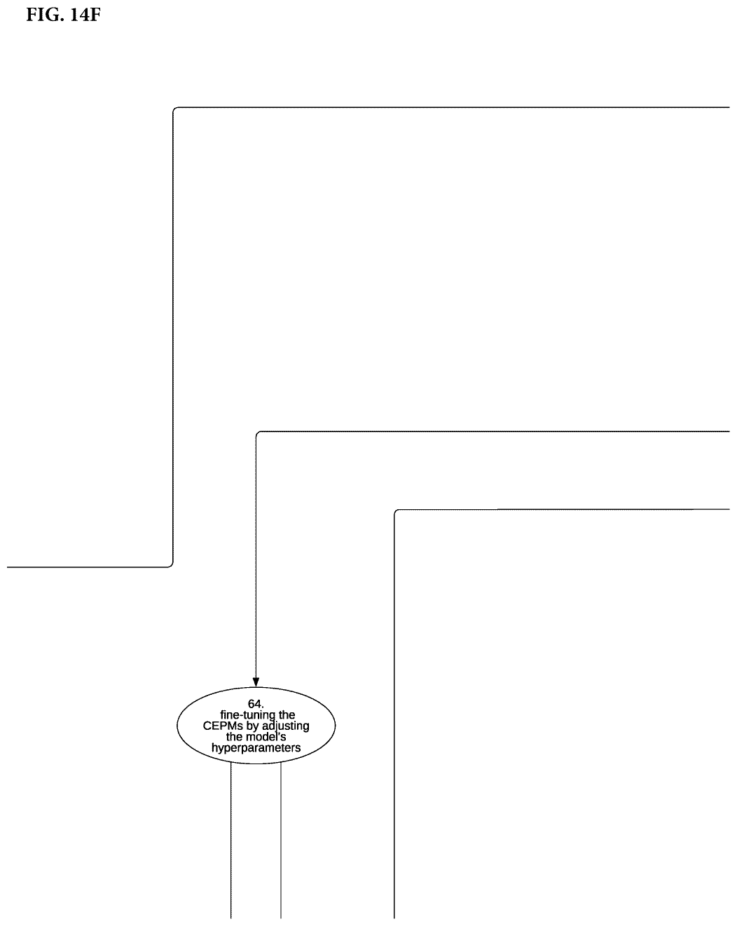

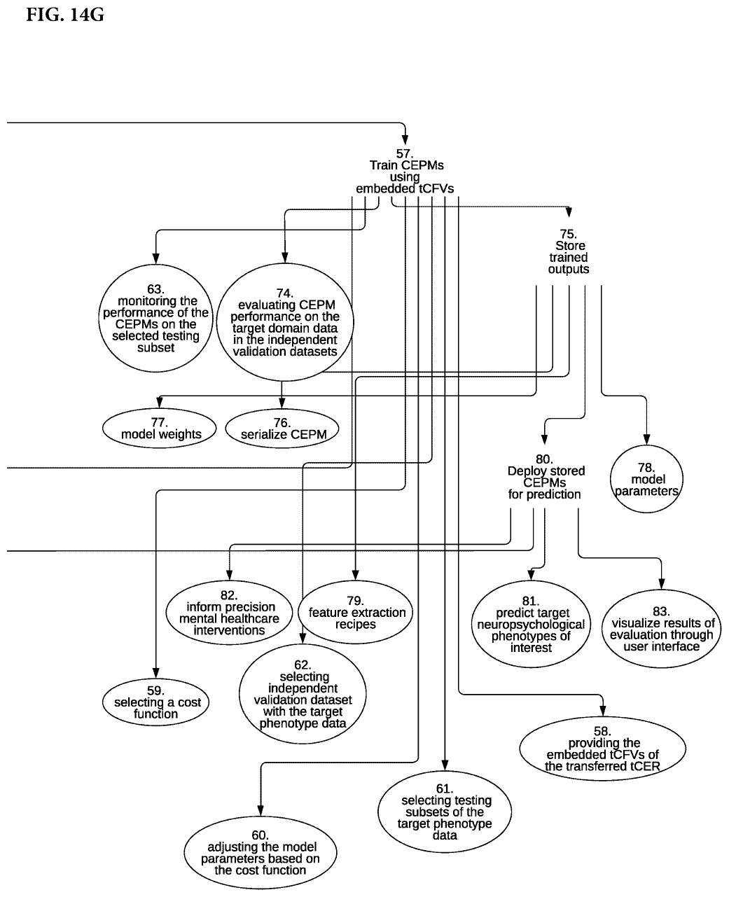

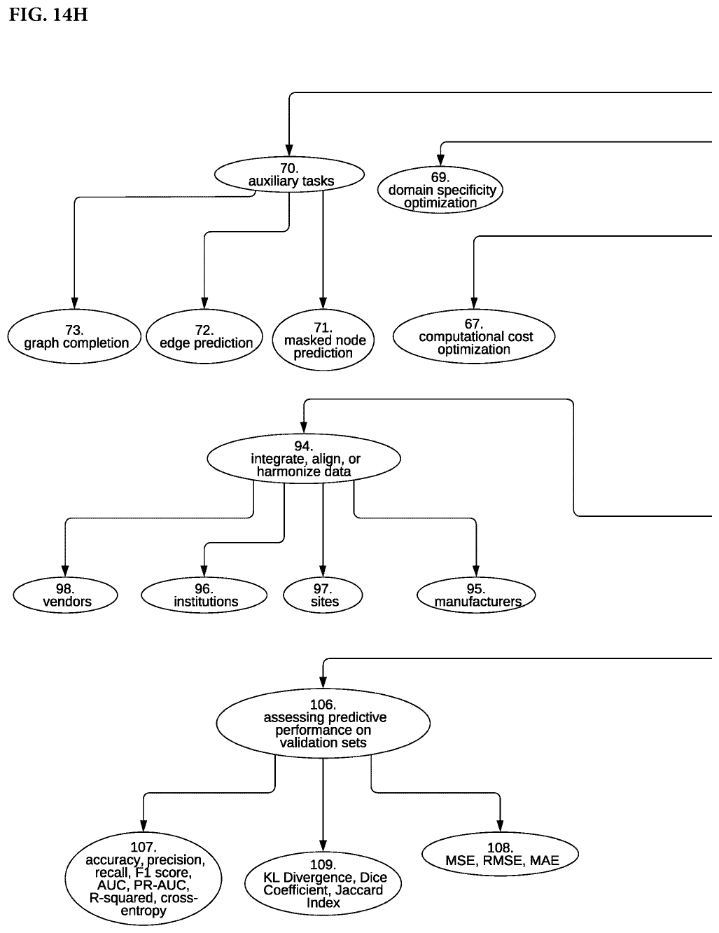

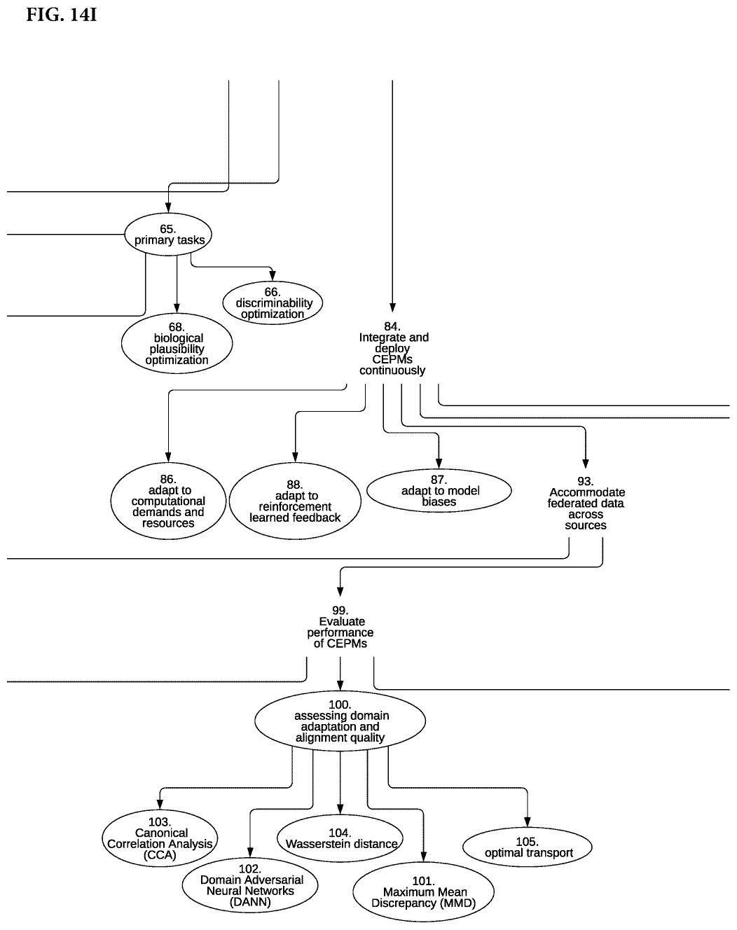

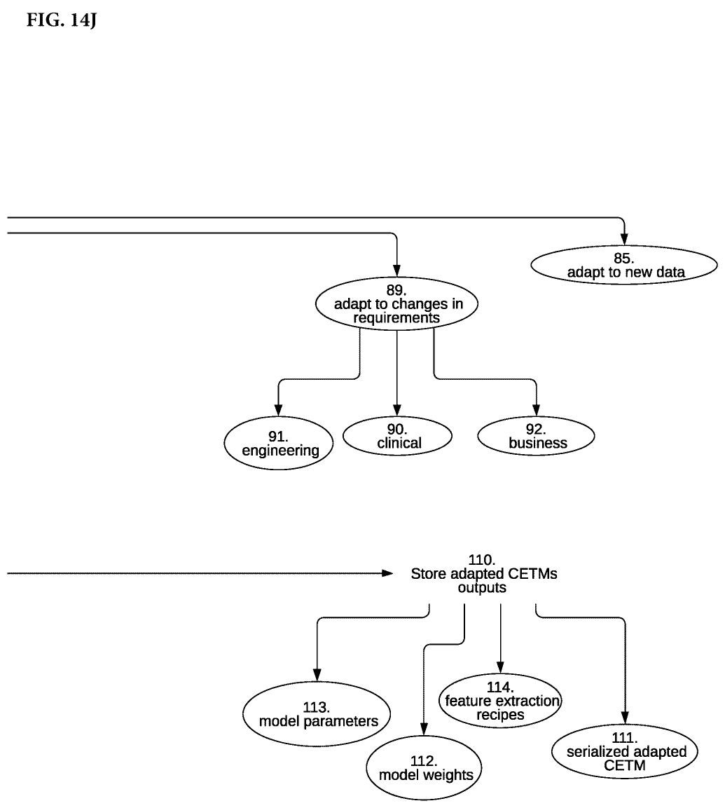

In some embodiments, at 102 in FIG. 1A, the method begins by selecting one or more target domains (1 in FIG. 14C), which comprise one or more neuropsychological phenotypes of interest (16 in FIG. 14C). At 104 in FIG. 1A, the method involves obtaining one or more modalities of target network connectivity data (TNCD) (14 in FIG. 14B) from one or more pluralities of individual subjects (15 in FIG. 14B), which constitute target subjects. In some embodiments, the one or more target neuropsychological phenotypes of interest may comprise one or more: diagnostic categories of neuropsychological disease; prognostic trajectories of neuropsychological disease (3 in FIG. 14B); symptom severity in one or more diagnostic categories of neuropsychological disease; neural, cognitive, behavioral, or emotional traits or states; neuropsychological treatment outcomes, response profiles (6-7 in FIG. 14B), or side-effect profiles (8 in FIG. 14B); biomarkers of neuropsychological disease (9 in FIG. 14C); neurodevelopmental milestones (10 in FIG. 14C); neurodegenerative stages (11 in FIG. 14B); genetic or environmental risk factors for neuropsychological disease; or cognitive or emotional abilities (12-13 in FIG. 14C).

At 106 in FIG. 1A, the method includes selecting one or more source domains (17 in FIG. 14B), which comprise one or more source phenotypes related to the target neuropsychological phenotypes of interest (24 in FIG. 14B). At 108 in FIG. 1A, the method involves obtaining one or more modalities of source network connectivity data (SNCD) from one or more pluralities of individual subjects, which constitute source subjects. In some embodiments, the one or more source phenotypes may comprise one or more: neuropsychological disease or treatment mechanisms (19 in FIG. 14B); risk or protective factors for neuropsychological disease (21 in FIG. 14B); or genetic, epigenetic, neural, molecular, physiological, social, demographic, environmental, developmental, cognitive, emotional, or behavioral traits or states (4-5 in FIG. 14C).

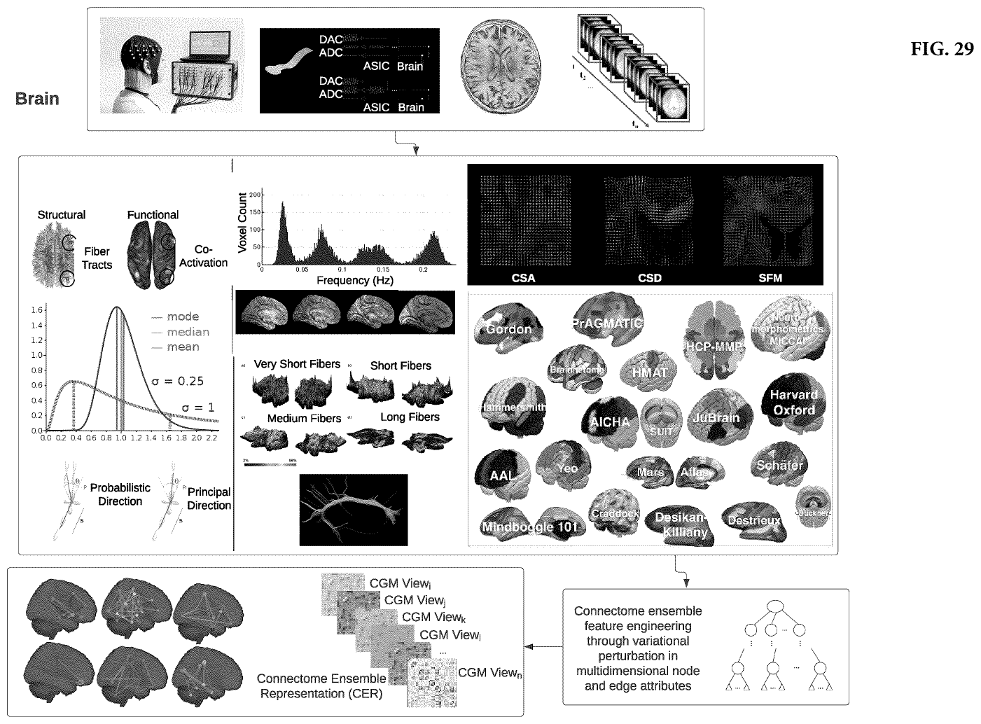

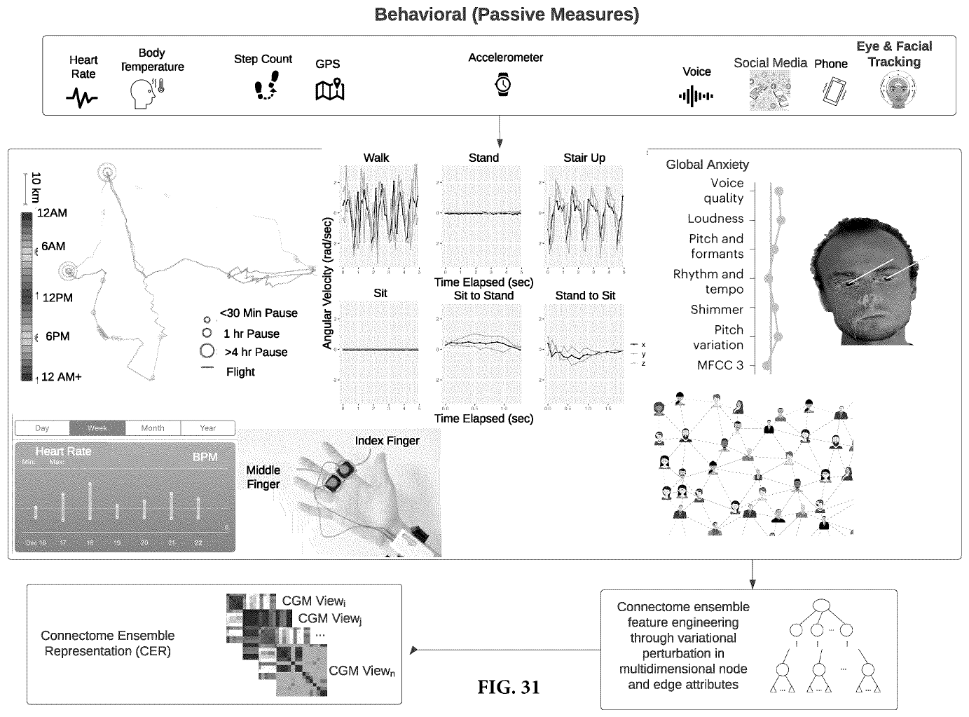

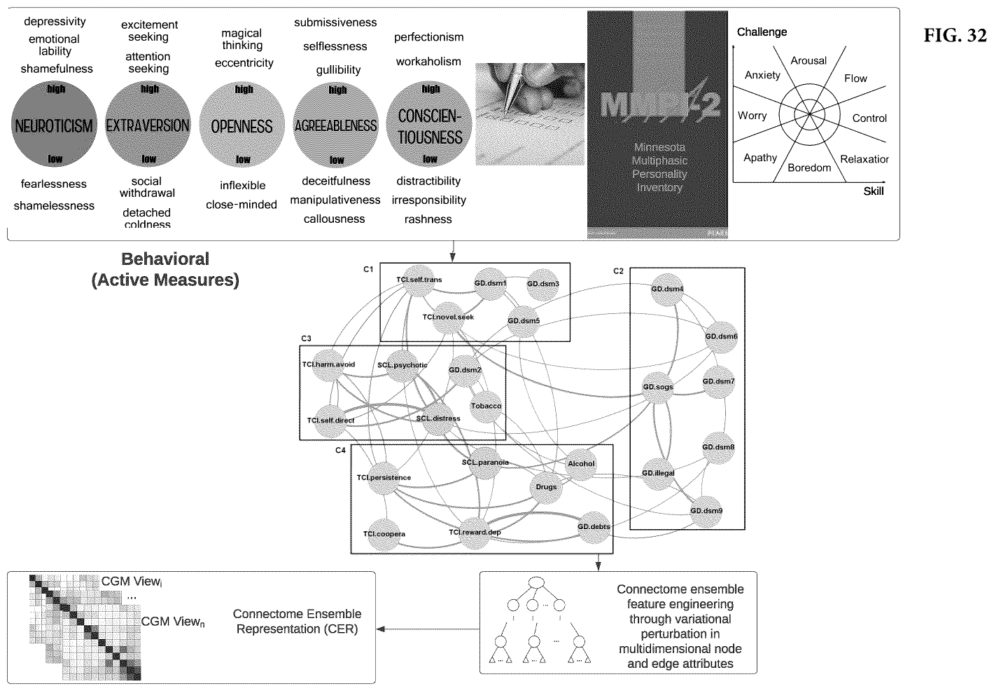

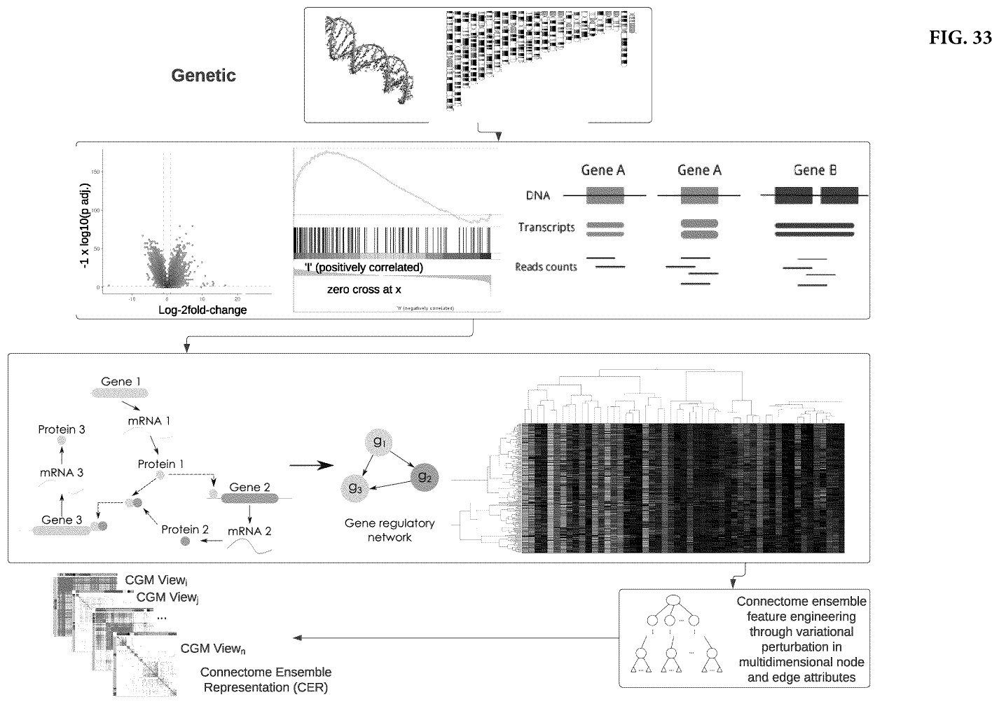

In some embodiments, at 110 in FIG. 1A, the method includes sampling, by a processing device, a plurality of source Connectome Graphical Models (sCGM) from the obtained SNCD for each source subject (25-26 in FIG. 14B). The processing device may comprise desktops or servers, further comprising central processors, graphics processors, tensor processors, or quantum processors. The sampling may involve one or more subprocesses of connectome ensemble feature engineering, which generates one or more pluralities of sCGM views by assigning one or more unique recipes of Network Connectivity Data Attributes (NCDA) (27 in FIG. 14B). The sampled one or more pluralities of sCGM views may constitute one or more source connectome ensemble representations (sCER) (28 in FIG. 14B). At 112 in FIG. 1A, the method includes assigning two or more unique recipes of Network Connectivity Data Attributes (NCDA) to two or more respective sCGMs, wherein each assigned unique recipe of NCDA constitutes a sCGM view. The modality-specific connectome ensemble feature engineering process is depicted for brain-based modality embodiments in FIG. 29, passive behavioral modality embodiments in FIG. 31, active behavioral modality embodiments in FIG. 32, and genetic modality embodiments in FIG. 33. In each embodiment, the raw data modality is depicted, followed by multiple further embodiments of multidimensional network connectivity data attributes, followed by sampling of respective CERs produced by decision-tree perturbation of multidimensional node and edge attributes.

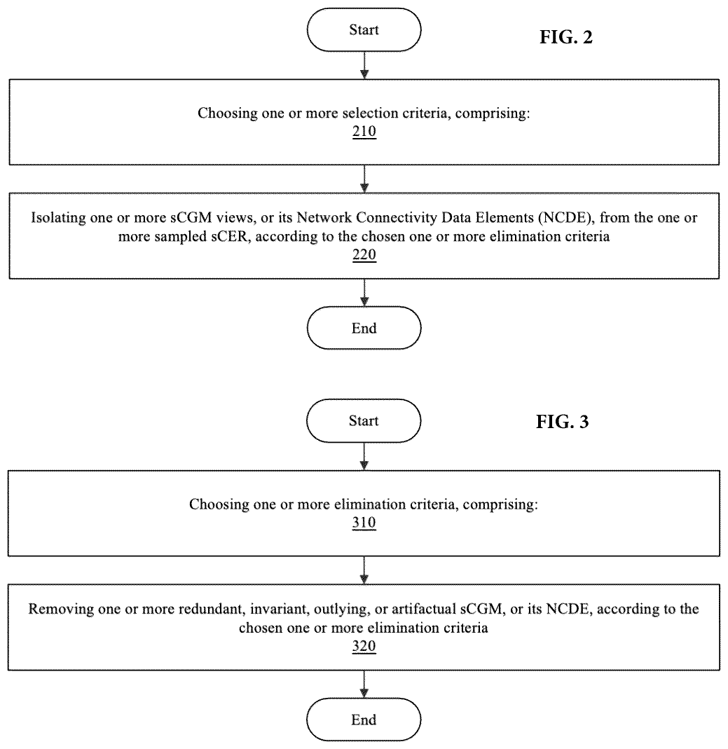

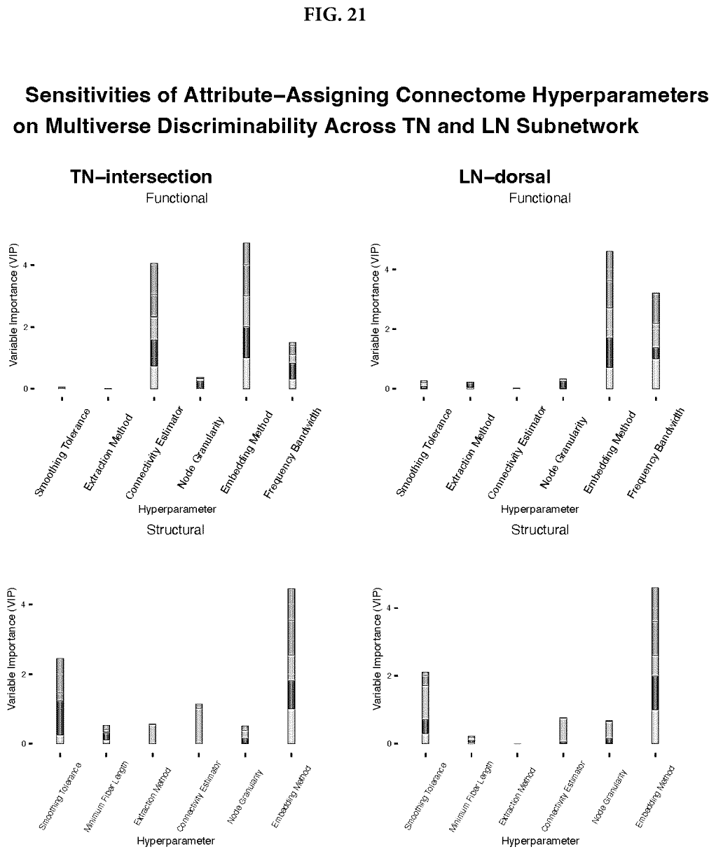

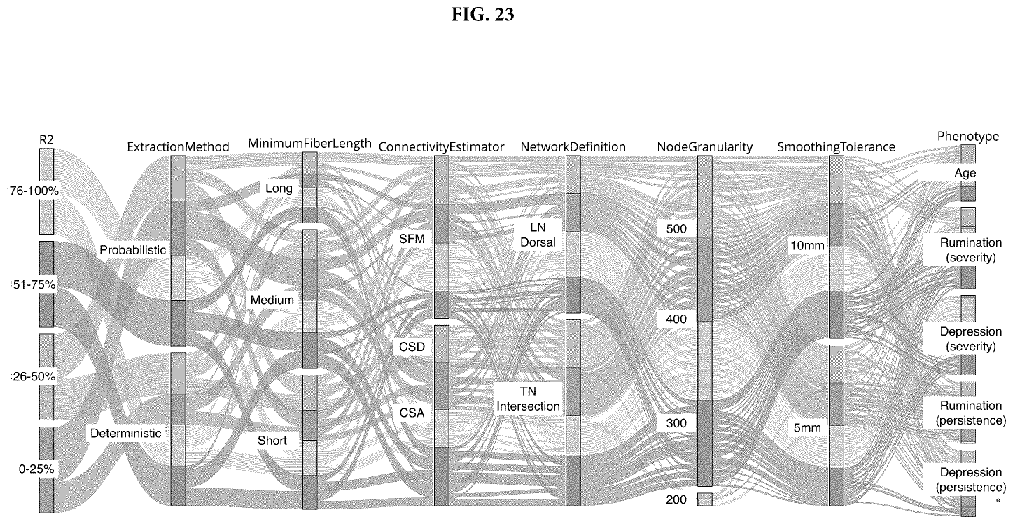

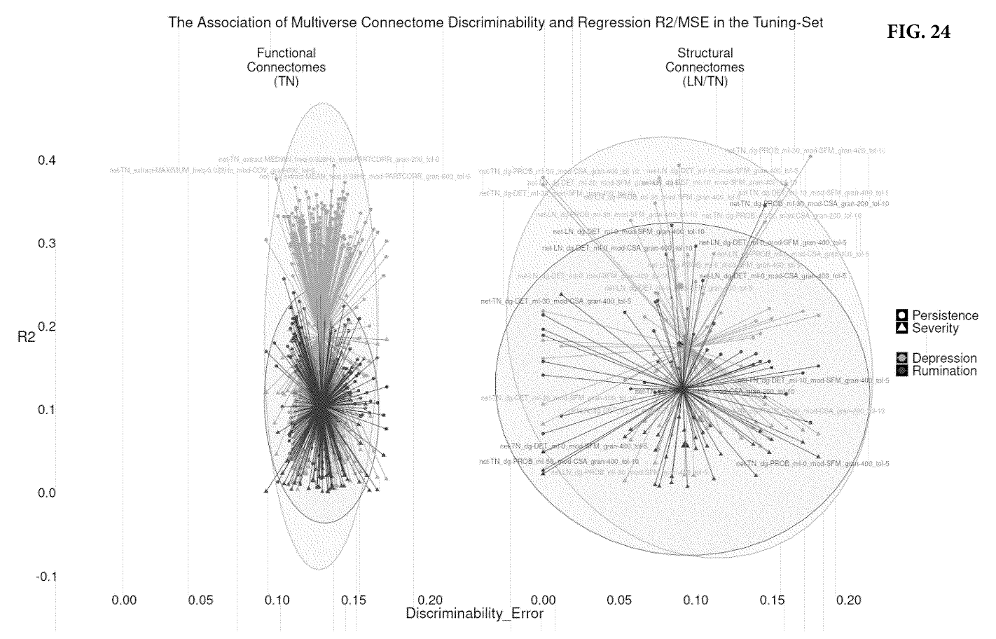

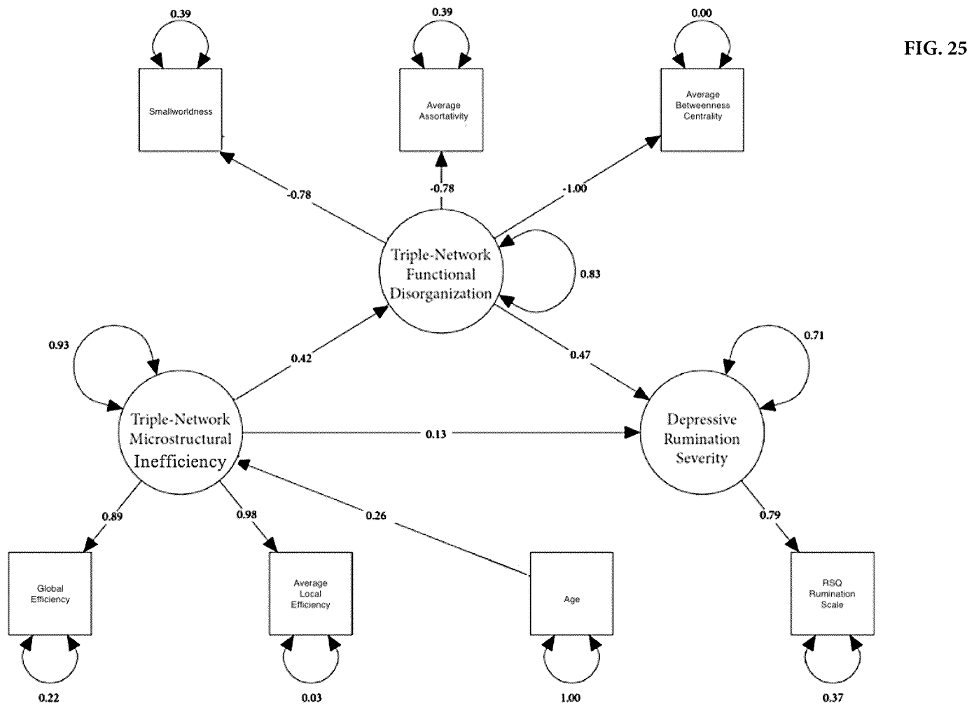

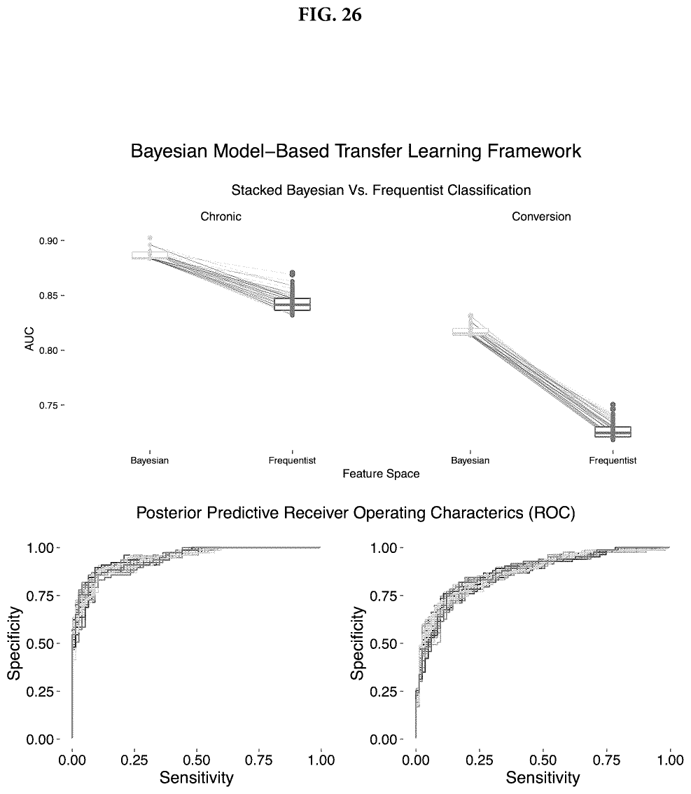

At 114 in FIG. 1A, the method includes pruning the one or more sampled sCER. In some embodiments, at 116 in FIG. 1B, the pruning includes eliminating, selecting, transforming, or otherwise reducing dimensionality of one or more sCGM views from the one or more sCER to produce one or more pruned sCERs (29-31 in FIG. 14E). In some embodiments, the pruning may be achieved through one or more intermediate analyses such as Bayesian analysis, auxiliary or multi-task learning, Multiverse Analysis (FIG. 24), Sensitivity Analysis (FIG. 21-FIG. 23), paired model comparisons (FIG. 26), Structural Equation Modeling (FIG. 25), or Factor Analysis. In some embodiments, pruning by selection further comprises choosing one or more selection criteria, at 210 in FIG. 2, which may include, for example, statistical significance, biological significance, clinical relevance, reproducibility, or computational efficiency; and isolating one or more sCGM views or its Network Connectivity Data Elements (NCDE) from the one or more sampled sCER, according to the chosen one or more selection criteria, at 220 in FIG. 2. In some embodiments, the pruning by elimination further comprises choosing one or more elimination criteria, at 310 in FIG. 3, which may include, for example, redundancy, invariance, outlying behavior, artifacts, or noise; and removing one or more sCGM views or its Network Connectivity Data Elements (NCDE) from the one or more sampled sCER, according to the chosen one or more elimination criteria, at 320 in FIG. 3. Other elimination criteria might include biological, statistical, or theoretical implausibility; domain unspecificity; computational, financial, or practical infeasibility; unreliability, invalidity, or indiscriminability; bias, unfairness, or inequity; unpredictability or unimportance; abnormality, invariance, uncertainty, sparsity, error, noise, or unrepresentativeness; or uninterpretability or unexplainability.

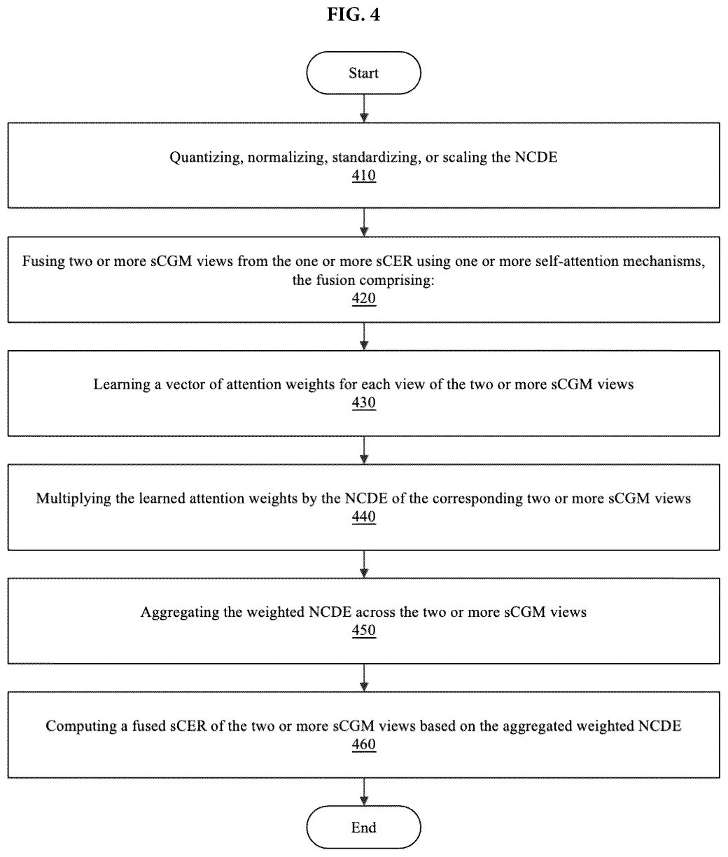

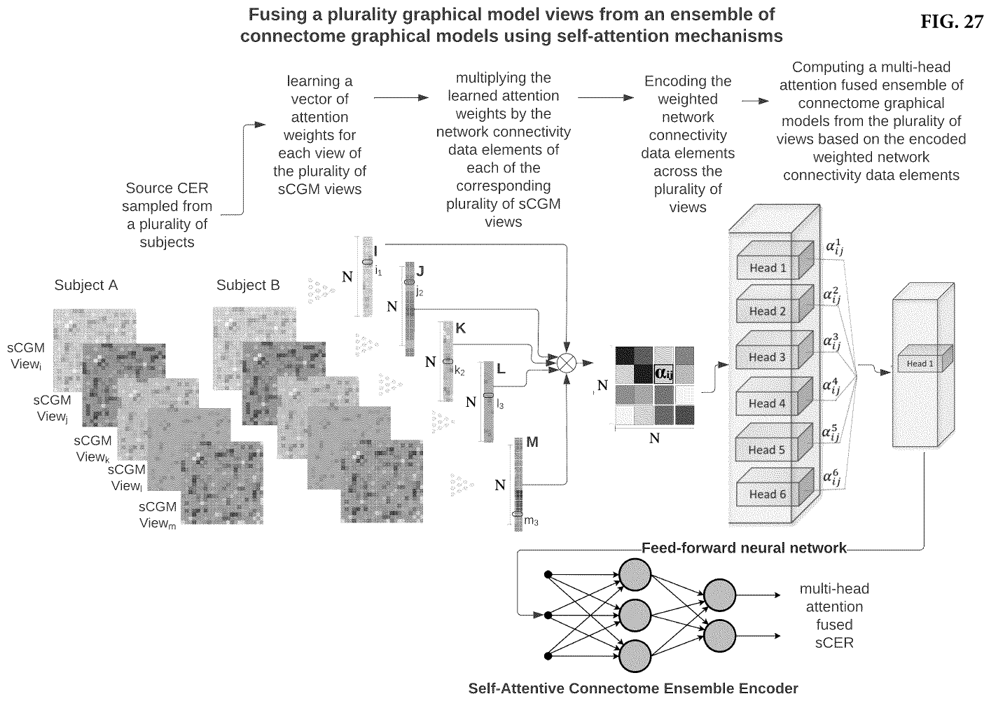

In some embodiments, pruning by transformation further comprises quantizing, normalizing, standardizing, or scaling the NCDE, at 410 in FIG. 4; and fusing two or more sCGM views from the one or more sCER using one or more self-attention mechanisms, the fusion, at 420 in FIG. 4, and in detail in FIG. 27. In some embodiments, the fusion further comprises learning a vector of attention weights for each view of the two or more sCGM views, at 430 in FIG. 4; multiplying the learned attention weights by the NCDE of the corresponding two or more sCGM views, at 440 in FIG. 4; aggregating the weighted NCDE across the two or more sCGM views, at 450 in FIG. 4; and computing a fused multi-head attention sCER of the two or more sCGM views based on the aggregated weighted NCDE, at 460 in FIG. 4.

At 118 in FIG. 1B, the method involves transferring the one or more pruned sCER from the one or more source domains to the one or more target domains (32-33 in FIG. 14D). At 120 in FIG. 1B, the method includes extracting, by the processing device, a plurality of target CGM (tCGM) (34 in FIG. 14D) from the obtained TNCD for each target subject (35 in FIG. 14D). The transferring involves re-assigning NCDA from pruned sCER to tCGMs (36 in FIG. 14D). This extraction may involve one or more subsequent subprocesses of connectome ensemble feature engineering to produce one or more target Connectome Ensemble Representations (tCER) (37 in FIG. 14D). At 122 in FIG. 1B, the method includes selecting one or more graph embedding algorithms (38 in FIG. 14D).

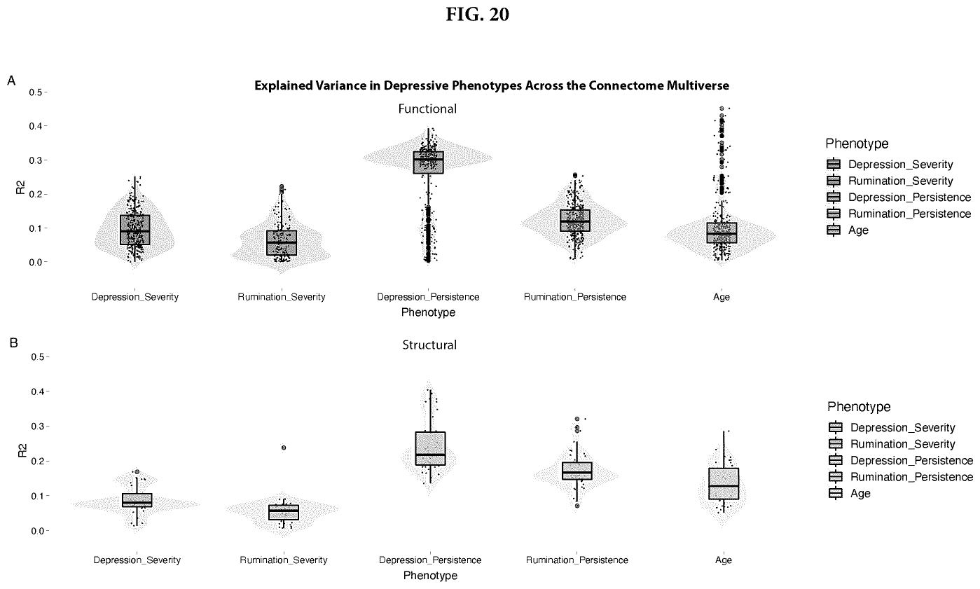

FIG. 9 is a flowchart that further describes the method from FIG. 1A, according to some embodiments of the present disclosure. At 902 in FIG. 9, the method includes evaluating the quality of domain adaptation and alignment (100 in FIG. 14I) by comparing the embedded source Connectome Feature Vectors (sCFVs) with the embedded target Connectome Feature Vectors (tCFVs) using one or more metrics, such as Maximum Mean Discrepancy (MMD), Domain Adversarial Neural Networks (DANN), Canonical Correlation Analysis (CCA), Wasserstein distance, or optimal transport (101-103 in FIG. 14I). At 910 in FIG. 9, the method involves refining the transfer process by adjusting the one or more source or target CERs, the pruning process, the embedding algorithms, the CEPM, or the cost function, based on the evaluated quality of domain adaptation and alignment. This refinement may include optimizing the discriminability (66 in FIG. 14H), computational cost (67 in FIG. 14H), biological plausibility, or domain specificity (FIG. 20) of the CEPM. In some embodiments, the refinement may involve fine-tuning the CEPM (64 in FIG. 14F) by adjusting the model's hyperparameters and performing one or more primary tasks, such as masked node prediction, edge prediction, or graph completion, at 920 in FIGS. 9 and 71-73 in FIG. 14H. The fine-tuning may also incorporate one or more auxiliary tasks (70 in FIG. 14H), which may be related to the primary tasks (66 in FIG. 14I) or provide additional information to improve the performance of the CEPM.

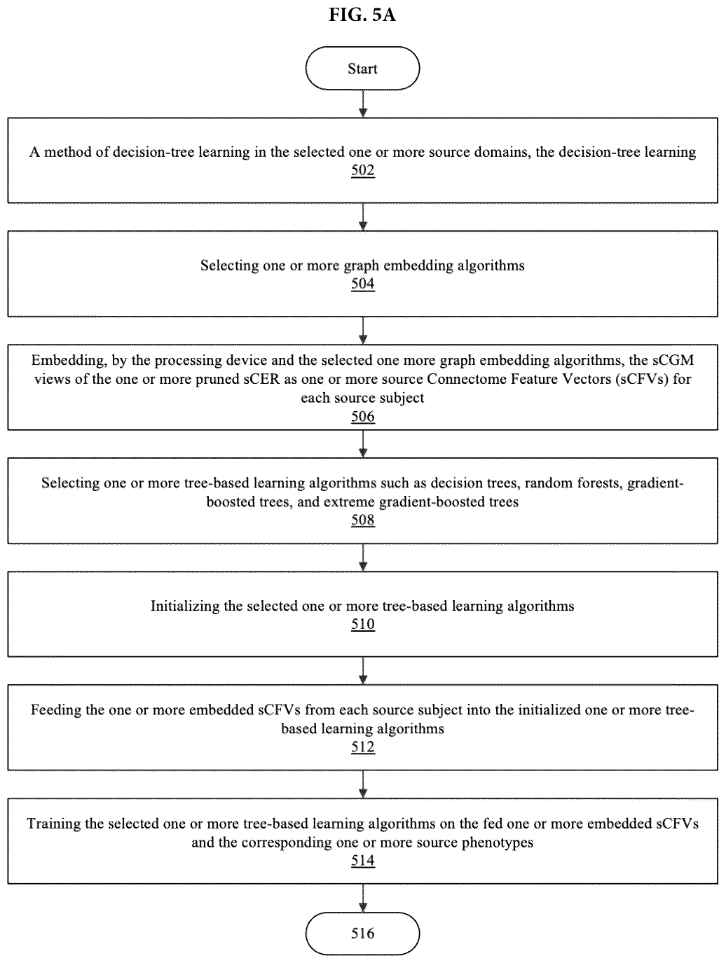

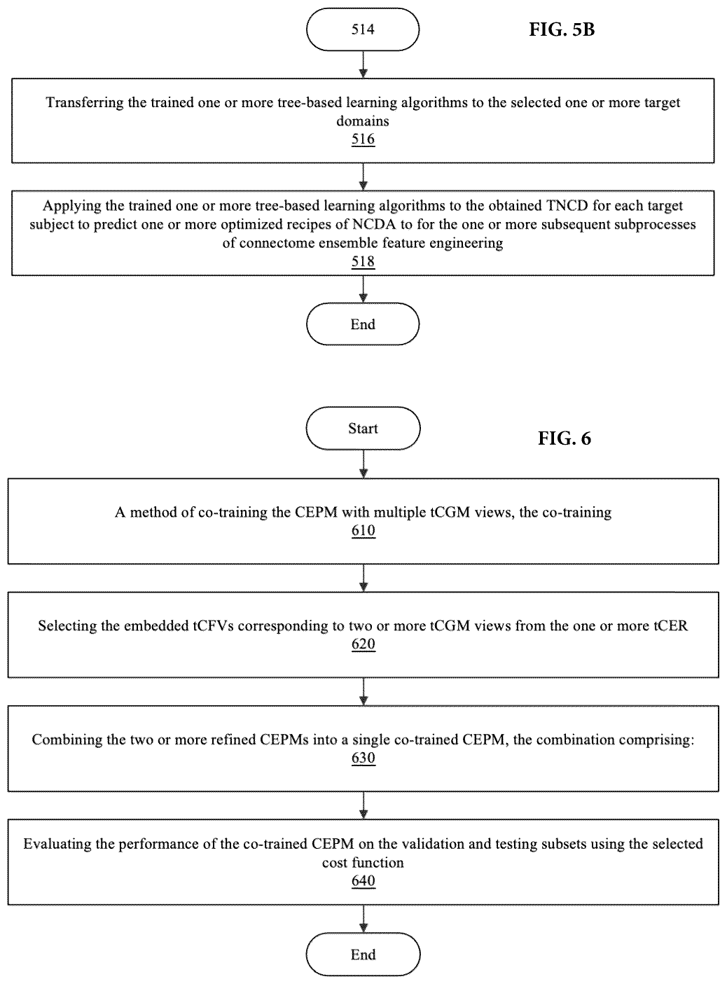

FIG. 5A to FIG. 5B are flowcharts that further describe the method from FIG. 1A, according to an embodiment of the present disclosure whereby the transferring alternatively comprises a method of decision-tree learning in the selected one or more source domains. At 502 in FIG. 5A, the method includes selecting one or more graph embedding algorithms. At 504 in FIG. 5A, the method involves embedding, by the processing device and the selected one more graph embedding algorithms, the sCGM views of the one or more pruned sCER as one or more source Connectome Feature Vectors (sCFVs) for each source subject. At 508 in FIG. 5A, the method includes selecting one or more tree-based learning algorithms such as decision trees, random forests, gradient-boosted trees, and extreme gradient-boosted trees. At 510 in FIG. 5A, the method involves initializing the selected one or more tree-based learning algorithms. At 512 in FIG. 5A, the method includes feeding the one or more embedded sCFVs from each source subject into the initialized one or more tree-based learning algorithms. At 514 in FIG. 5A, the method involves training the selected one or more tree-based learning algorithms on the fed one or more embedded sCFVs and the corresponding one or more source phenotypes. At 516 in FIG. 5B, the method includes transferring the trained one or more tree-based learning algorithms to the selected one or more target domains. At 518 in FIG. 5B, the method involves applying the trained one or more tree-based learning algorithms to the obtained TNCD for each target subject to predict one or more optimized recipes of NCDA for the one or more subsequent subprocesses of connectome ensemble feature engineering.

FIG. 6 is a flowchart that further describes the method from FIG. 1A, according to an embodiment of the present disclosure whereby the transferring alternatively comprises a method of co-training the CEPM with multiple tCGM views. At 610 in FIG. 6, the method includes selecting the embedded tCFVs corresponding to two or more tCGM views from the one or more tCER. At 620 in FIG. 6, the method involves refining the CEPM for each of the two or more tCGM views independently, using the corresponding embedded tCFVs and the corresponding one or more target neuropsychological phenotypes of interest. At 630 in FIG. 6, the method includes combining the two or more refined CEPMs into a single co-trained CEPM. The combination may involve one or more techniques, such as averaging the weights of the two or more refined CEPMs, stacking their outputs, or using an ensemble learning method. At 640 in FIG. 6, the method involves evaluating the performance of the co-trained CEPM on the validation and testing subsets using the selected cost function, wherein a satisfactory performance indicates the effectiveness of the co-trained CEPM in predicting the one or more target neuropsychological phenotypes of interest.

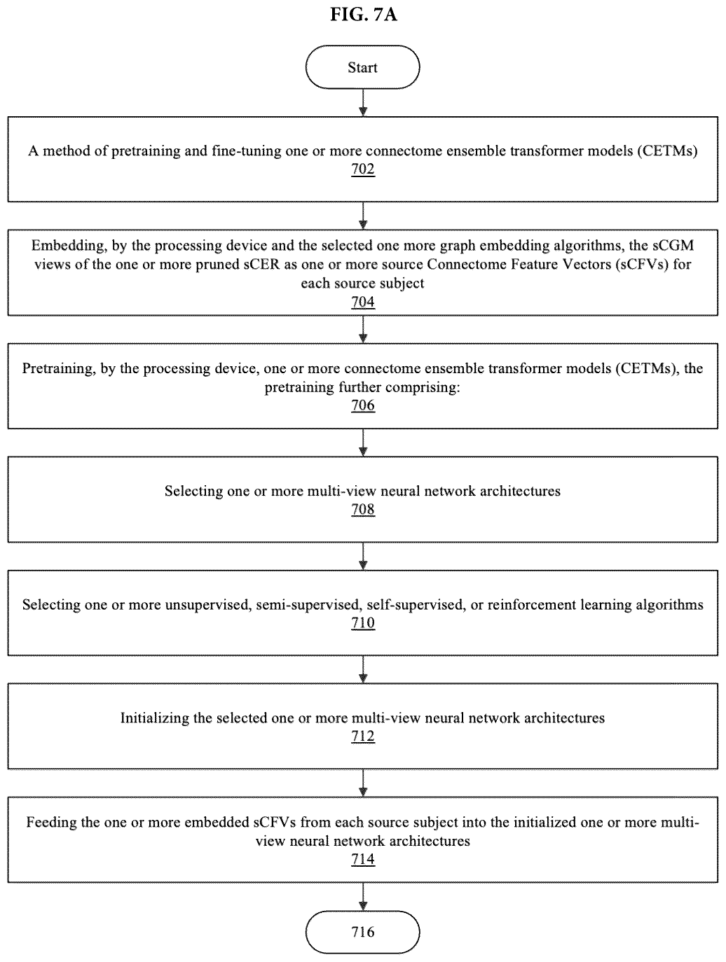

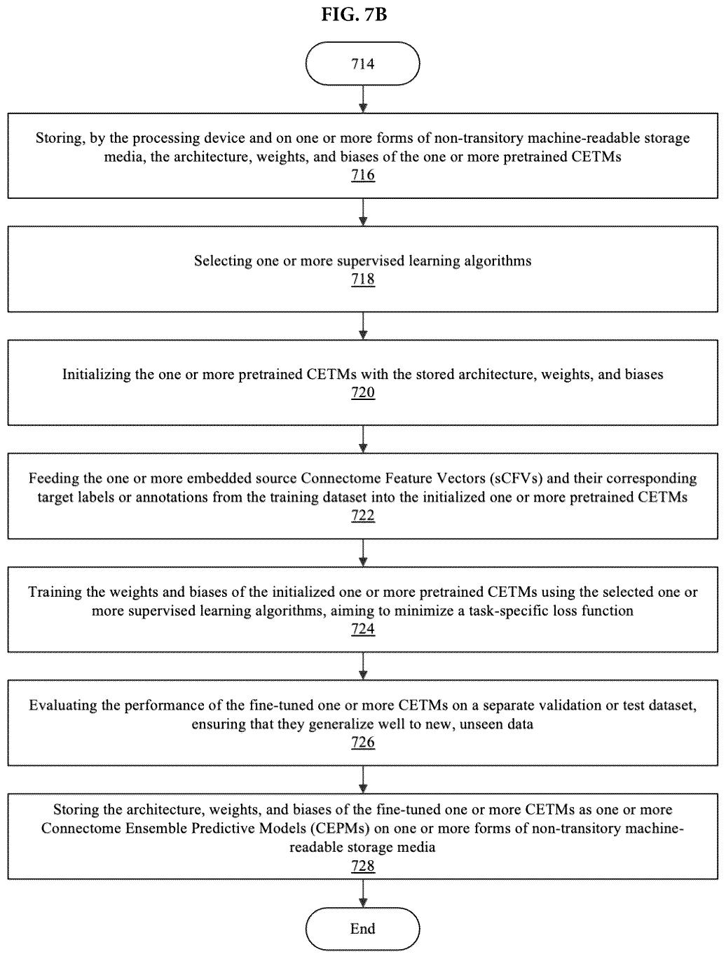

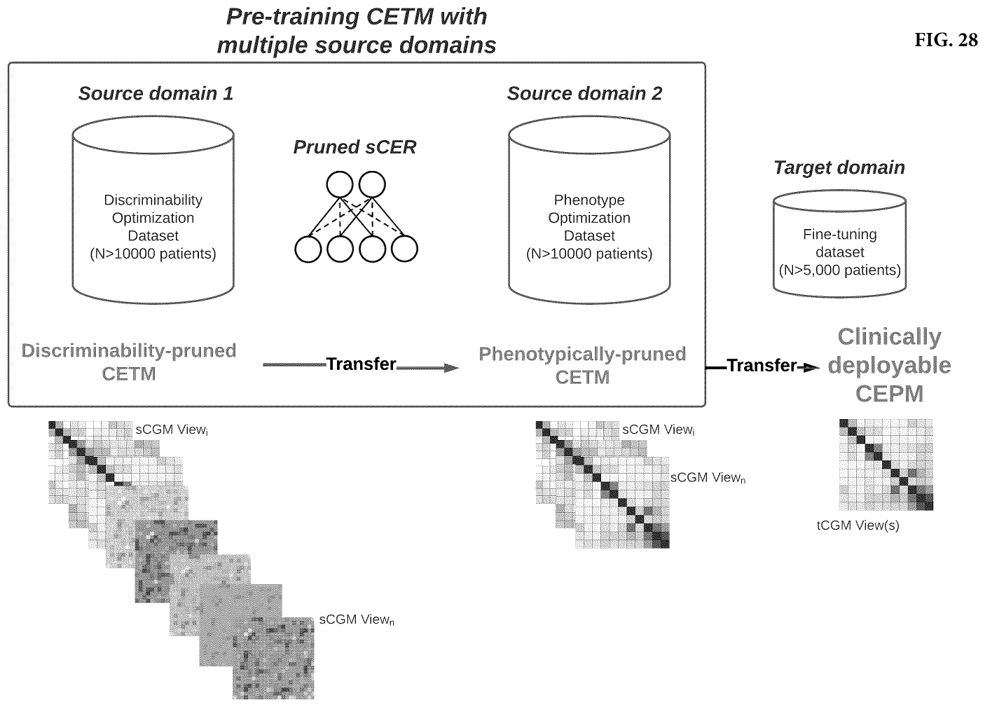

FIG. 7A to FIG. 7B are flowcharts that further describe the method from FIG. 1A, whereby the transferring alternatively comprises a method of pretraining and fine-tuning (50 in FIG. 14D) one or more connectome ensemble transformer models (CETMs) (FIG. 28). At 702 in FIG. 7A, the method includes selecting one or more graph embedding algorithms. At 704 in FIG. 7A, the method involves embedding, by the processing device and the selected one more graph embedding algorithms, the sCGM views of the one or more pruned sCER as one or more source Connectome Feature Vectors (sCFVs) for each source subject. At 706 in FIG. 7A, the method includes pretraining, by the processing device, one or more connectome ensemble transformer models (CETMs), which involves selecting one or more multi-view neural network architectures at 708 in FIG. 7A, and selecting one or more unsupervised, semi-supervised, self-supervised, or reinforcement learning algorithms at 710 in FIG. 7A and 51-56 in FIG. 7A in FIG. 14E. At 712 in FIG. 7A, the method involves initializing the selected one or more multi-view neural network architectures. At 714 in FIG. 7A, the method includes feeding the one or more embedded sCFVs from each source subject into the initialized one or more multi-view neural network architectures. At 716 in FIG. 7B, the method involves storing, by the processing device and on one or more forms of non-transitory machine-readable storage media, the architecture, weights, and biases of the one or more pretrained CETMs (110-111 in FIG. 14J). At 718 in FIG. 7B, the method includes selecting one or more supervised learning algorithms. At 720 in FIG. 7B, the method involves initializing the one or more pretrained CETMs with the stored architecture, weights, and biases. At 722 in FIG. 7B, the method includes feeding the one or more embedded source Connectome Feature Vectors (sCFVs) and their corresponding target labels or annotations from the training dataset into the initialized one or more pretrained CETMs. At 724 in FIG. 7B, the method involves training the weights and biases of the initialized one or more pretrained CETMs using the selected one or more supervised learning algorithms, aiming to minimize a task-specific loss function. The task-specific loss function may comprise one or more objectives related to the prediction of the target neuropsychological phenotypes of interest in the target domain. At 726 in FIG. 7B, the method includes evaluating the performance of the fine-tuned one or more CETMs on a separate validation or test dataset, ensuring that they generalize well to new, unseen data. This evaluation may involve computing one or more performance metrics, such as accuracy, precision, recall, F1 score, AUC, R-squared, or other suitable metrics. At 728 in FIG. 7B, the method involves storing the architecture, weights, and biases of the fine-tuned one or more CETMs as one or more Connectome Ensemble Predictive Models (CEPMs) on one or more forms of non-transitory machine-readable storage media.

In some embodiments, at 124 in FIG. 1B, the method includes embedding, by the processing device and the selected one or more graph embedding algorithms, the transferred one or more tCER from each target subject, as one or more target Connectome Feature Vectors (tCFVs) for each target subject (44 in FIG. 14E). Embeddings can either be performed on each tCGM at a time (39 in FIG. 14D) or on an omnibus grouping of tCGM simultaneously (40 in FIG. 14D), which can include a complete set, subset, or multiple subsets (41-43 in FIG. 14D). The embedding projects the re-assigned NCDA of the extracted plurality of tCGM views into one or more lower-dimensional feature-spaces (45 in FIG. 14E). In some embodiments, the one or more tCER or its embedded tCFV may be represented as one or more respective multigraphical or hypergraphical models. The one or more transferred multigraphical models may comprise a plurality of multiplicatively attributed tCGM views that form separable graph layers, the nodes of which may be aligned or matched. The one or more transferred hypergraphical models may encode one or more pairwise similarity relationships between one or more pairs of tCFV constituents of the one or more tCER. The CEPM may be trained on the one or more multigraphical or hypergraphical models.

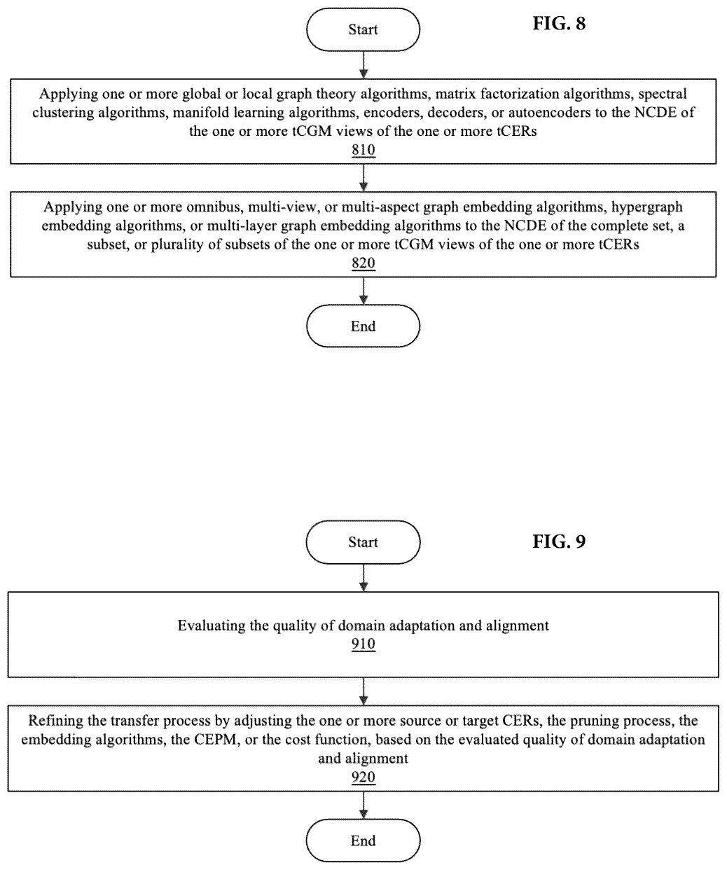

FIG. 8 is a flowchart that further describes the method from FIG. 1A, according to some embodiments of the present disclosure, whereby the embedding comprises selecting one or more graph embedding algorithms that are applicable to single CGM constituents of the CER or omnibus CGM constituents of the CER. At 802 in FIG. 8, the method includes applying one or more global or local graph theory algorithms, matrix factorization algorithms, spectral clustering algorithms, manifold learning algorithms, encoders, decoders, or autoencoders to the NCDE of the one or more tCGM views of the one or more tCERs. At 810 in FIG. 8, the method involves applying one or more omnibus, multi-view, or multi-aspect graph embedding algorithms, hypergraph embedding algorithms, or multi-layer graph embedding algorithms to the NCDE of the complete set, a subset, or plurality of subsets of the one or more tCGM views of the one or more tCERs. The selected one or more graph embedding algorithms may be chosen based on their suitability for the specific types of tCGM views and the target neuropsychological phenotypes of interest.

At 126 in FIG. 1B, the method includes selecting one or more machine learning models in the target domain (44 in FIG. 14E), wherein the selected one or more machine learning models at least partially consume the transferred and embedded one or more tCFVs from each target subject. The selected one or more machine learning models constitute a Connectome Ensemble Predictive Model (CEPM) (46 in FIG. 14E). In some embodiments, these models can be a variety of types (47 in FIG. 14D), broadly including discriminative (48 in FIG. 14D) or generative (49 in FIG. 14D) models.

At 128 in FIG. 1B, the method involves splitting the obtained one or more pluralities of target subjects into training, testing, and validation subsets (61-62 in FIG. 14G). At 130 in FIG. 1C, the method includes selecting a cost function, wherein the selected cost function evaluates the performance of the selected CEPM and is optimized during the training process. At 132 in FIG. 1C, the method involves selecting an optimization algorithm, wherein the selected optimization algorithm updates the parameters of the selected CEPM to minimize the selected cost function.

In some embodiments, at 134 in FIG. 1C, the method includes feeding the training subset of the obtained one or more pluralities of target subjects into the selected CEPM (58 in FIG. 14G). The training subset comprises the embedded tCFVs of each transferred tCGM view (57 in FIG. 14G) and the corresponding one or more target neuropsychological phenotypes of interest. The feeding process further comprises recursively partitioning the training subset. At 136 in FIG. 1C, the method involves initializing the selected CEPM. At 138 in FIG. 1C, the method includes adjusting the parameters of the initialized CEPM based on the selected optimization algorithm and the selected cost function (60 in FIG. 14G).

At 140 in FIG. 1C and 99 in FIG. 14I, the method involves validating, by the processing device and for each tCGM view, the trained CEPM in the target domain. The validation process includes feeding the validation subset of the obtained one or more pluralities of target subjects into the trained CEPM, wherein the validation subset comprises the embedded tCFVs of each transferred tCGM view and the corresponding one or more target neuropsychological phenotypes of interest. At 142 in FIG. 1C, the method includes evaluating the performance of the trained CEPM on the validation subset using the selected cost function, wherein a satisfactory performance indicates that the training performance of the CEPM generalizes to unseen data.

In some embodiments, at 146 in FIG. 1D, the method includes adjusting, when applicable, machine-learning hyperparameters of the selected CEPM, based on the performance on the validation subset. The adjusting process may comprise a grid search, random search, or Bayesian optimization of the machine-learning hyperparameters. At 148 in FIG. 1D, the method involves selecting the one or more tCGM views with the best performance on the validation subset, based on the selected cost function, wherein the selected one or more tCGM views constitute the optimal tCER.

At 150 in FIG. 1D, the method includes feeding the testing subset of the obtained one or more pluralities of target subjects into the trained and validated CEPM, wherein the testing subset comprises the embedded tCFVs and the corresponding one or more target neuropsychological phenotypes of interest. At 152 in FIG. 1D, the method involves evaluating the performance of the trained and validated CEPM on the testing subset using the selected cost function (59 in FIG. 14G), wherein a satisfactory performance indicates the effectiveness of the CEPM in predicting the one or more target neuropsychological phenotypes of interest. Final evaluation is performed with respect to the validation set (106 in FIG. 14H), and can be assessed using performance measures such as accuracy, precision, recall, F1-score, AUC, PR-AUC, R-squared, MSE, MAE, or cross entropy for discriminative models (107 in FIG. 14H), or KL Divergence, Dice Coefficient, Jaccard Index for generative models (109 in FIG. 14H).

In some embodiments, at 154 in FIG. 1D, the method includes storing, on one or more forms of non-transitory machine-readable storage media, the one or more embedded and trained outputs. The outputs comprise the weights, biases/parameters of the one or more trained CEPM (78 in FIG. 14G), the unique recipes of NCDA from the selected optimal tCER, and the selected one or more embedding algorithms. At 156 in FIG. 1D, the method involves deploying the stored one or more stored outputs in the target domain to predict the one or more neuropsychological phenotypes of interest. In some embodiments, the stored outputs may be provided to a user interface or user experience that allows the user to interact with the CEPM, monitor the training, and visualize the results of the evaluation (83 in FIG. 14G).

In some embodiments, the trained, validated, and stored CEPM may be integrated and deployed continuously (84 in FIG. 14I) within a health care, research, or industry setting. This integration may involve updating the CEPM with new data, monitoring the performance of the CEPM, and adjusting the CEPM based on feedback from users and experts. The CEPM may be integrated with various data sources, such as electronic health records, research databases, and imaging repositories, to enhance its predictive capabilities and align it with the specific needs of the health care, research, or industry setting. The integration process may involve collaborating with vendors (98 in FIG. 14H), institutions (96 in FIG. 14H), sites (97 in FIG. 14H), and manufacturers (95 in FIG. 14H) to ensure seamless and efficient deployment of the CEPM.

The deployed CEPM may adapt to computational demands and resources (86 in FIG. 14I) within the health care, research, or industry setting, enabling it to scale and perform efficiently in various computing environments. This may involve optimizing the CEPM for different types of processing devices, such as central processors, graphics processors, tensor processors, or quantum processors, and adjusting the CEPM to accommodate different levels of computational power or storage capacity.

The deployed CEPM may also adapt to reinforcement learned feedback (88 in FIG. 14I) provided by users, experts, or automated systems, allowing it to continuously improve its performance and align with the specific needs and objectives of the health care, research, or industry setting. This may involve incorporating feedback into the cost function or training process, adjusting the weights and biases of the CEPM, and updating the CEPM with new data or features.

Furthermore, the deployed CEPM may adapt to model biases (87 in FIG. 14I), ensuring that the predictions it generates are fair, unbiased, and representative of the target population. This may involve identifying and addressing potential sources of bias in the training data, the CEPM architecture, or the optimization process, and implementing techniques to mitigate or eliminate these biases.

The deployed CEPM may also accommodate federated data across sources (93 in FIG. 14I), enabling it to learn from diverse, distributed datasets while preserving the privacy and security of individual data points. This may involve implementing federated learning, secure multi-party computation, or homomorphic encryption techniques to train and update the CEPM in a decentralized manner, without requiring direct access to raw data from each source.

Additionally, the deployed CEPM may adapt to changes in requirements (89 in FIG. 14J), such as shifts in the target neuropsychological phenotypes of interest, new data sources, or evolving regulatory or ethical guidelines. This may involve updating the CEPM to incorporate new data, features, or objectives, and ensuring that it remains compliant with the relevant standards and best practices within the health care, research, or industry setting.

Finally, the deployed CEPM may adapt to new data (85 in FIG. 14J) as it becomes available, allowing it to stay up-to-date and maintain its predictive accuracy over time. This may involve incorporating new data into the training, validation, or testing subsets, retraining the CEPM as needed, and continuously evaluating its performance using the selected cost function and performance metrics. The CEPM may also be adapted to predict clinical (90 in FIG. 14J) or business-related outcomes (92 in FIG. 14J), depending on the specific needs and objectives of the health care, research, or industry setting.

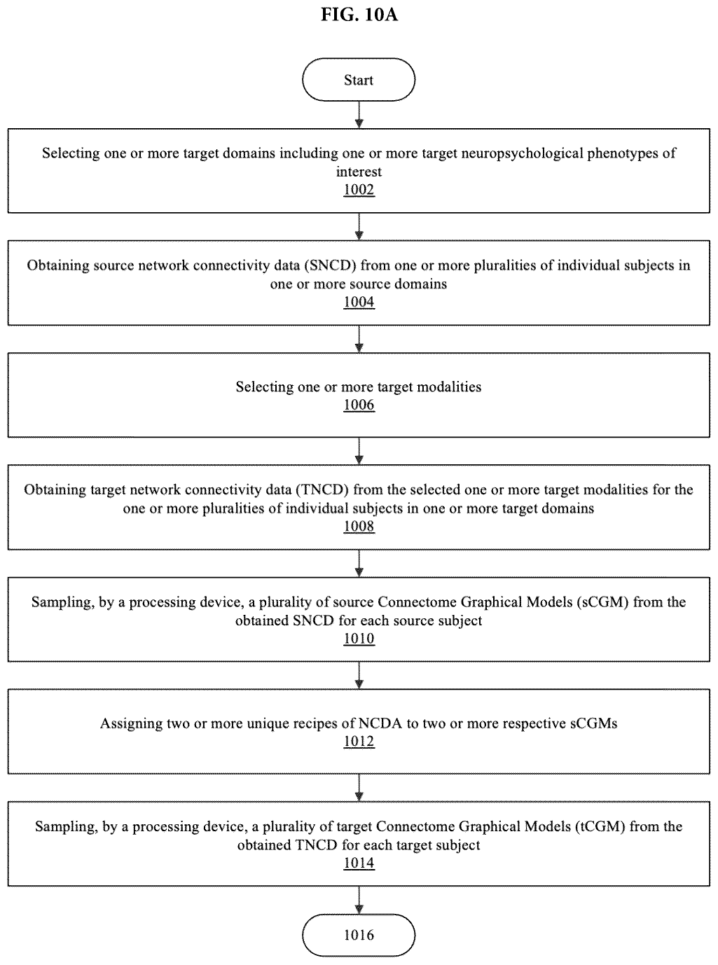

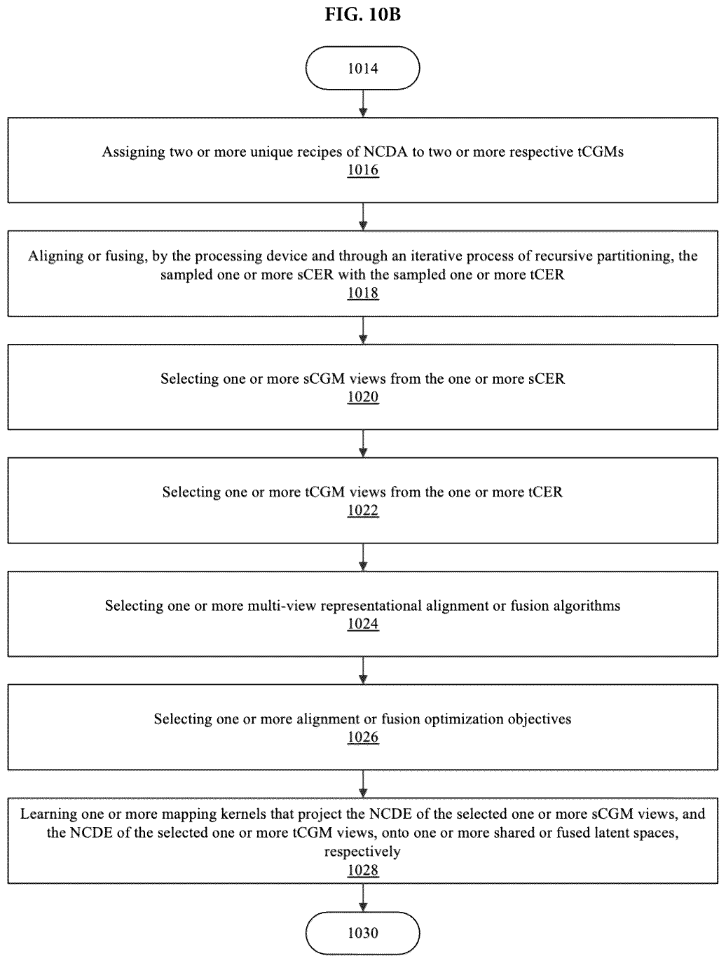

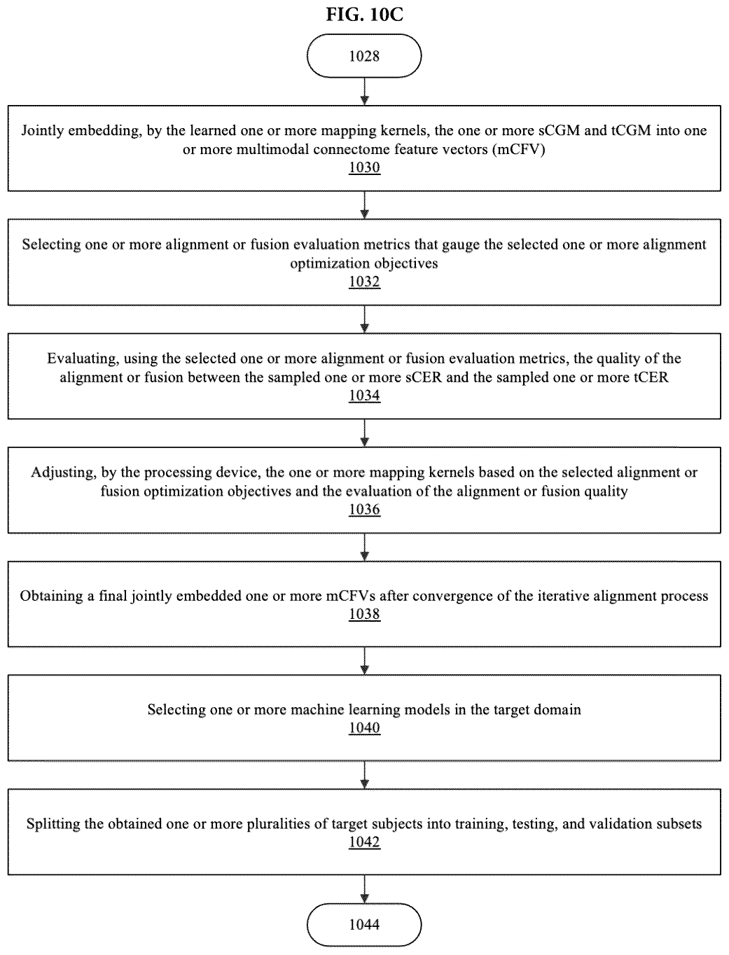

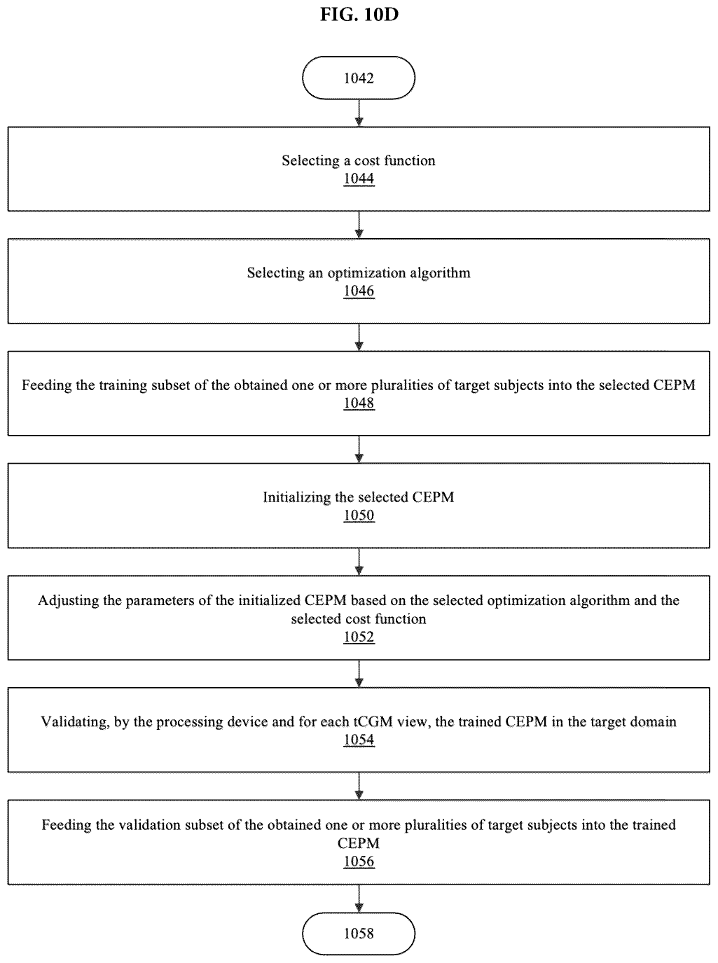

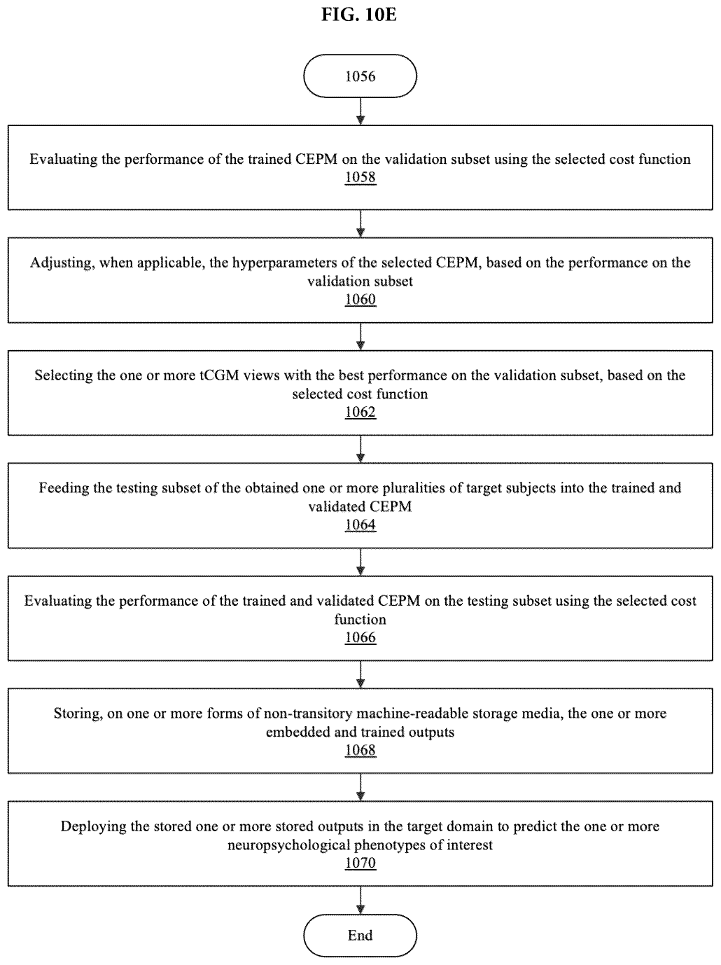

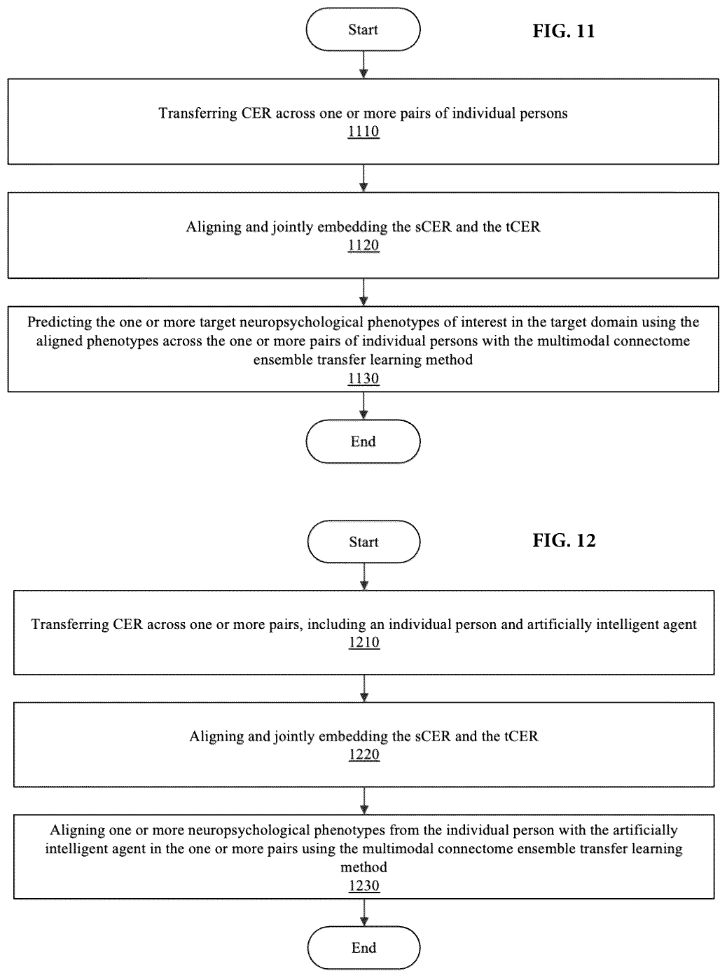

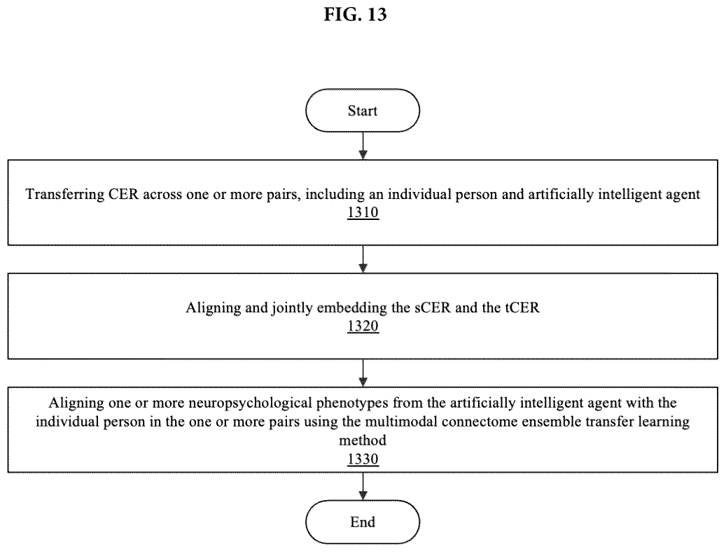

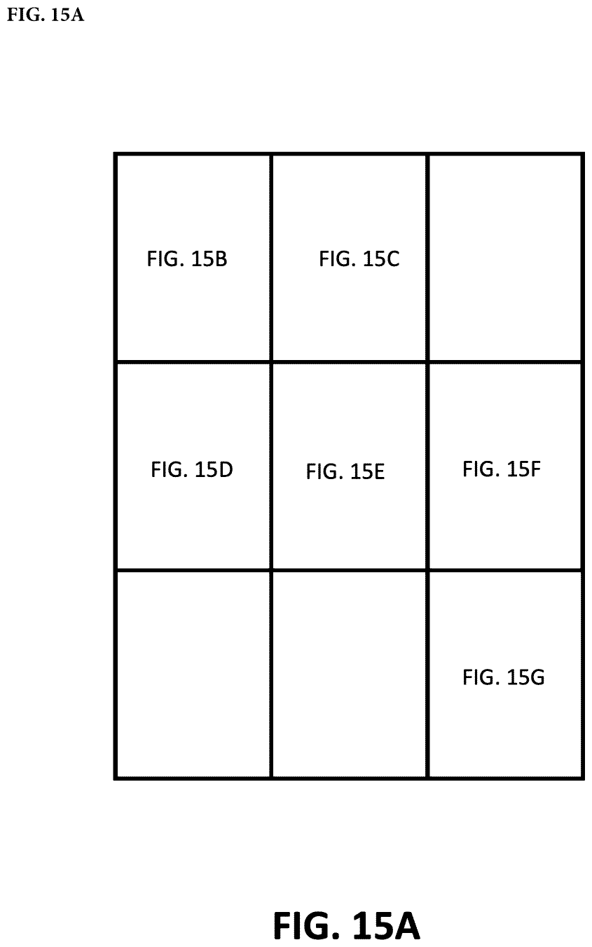

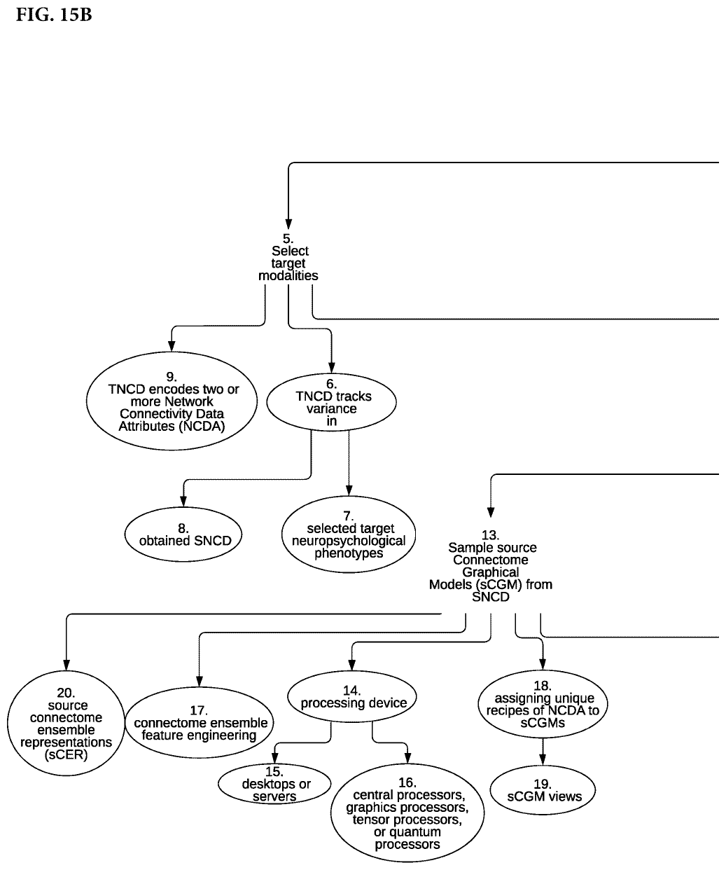

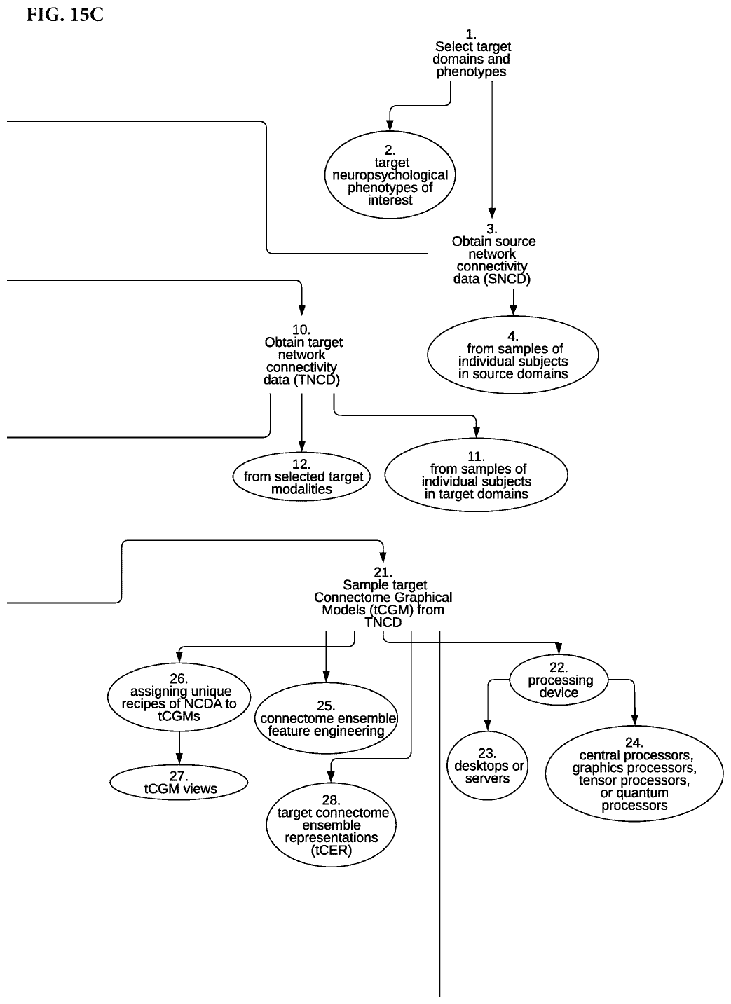

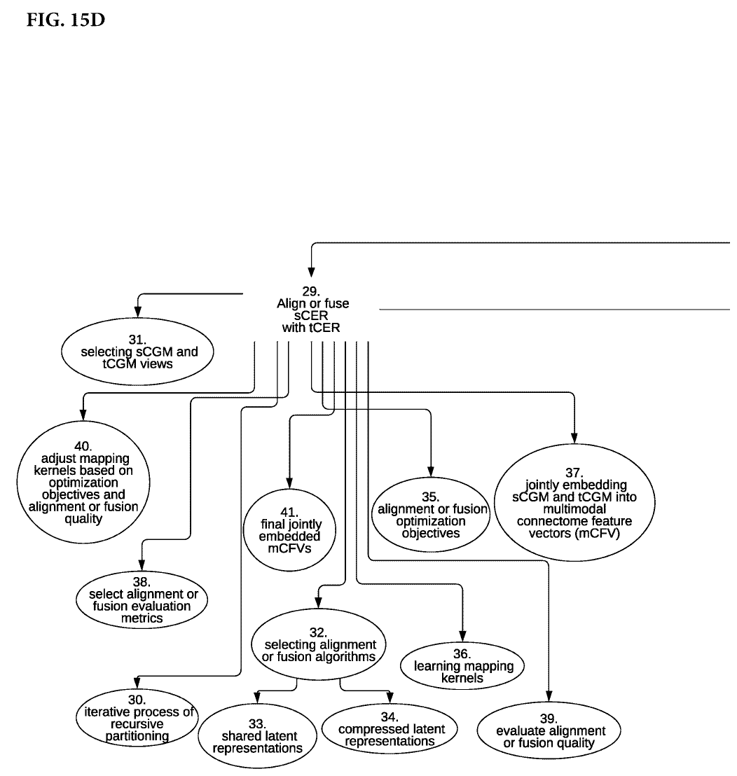

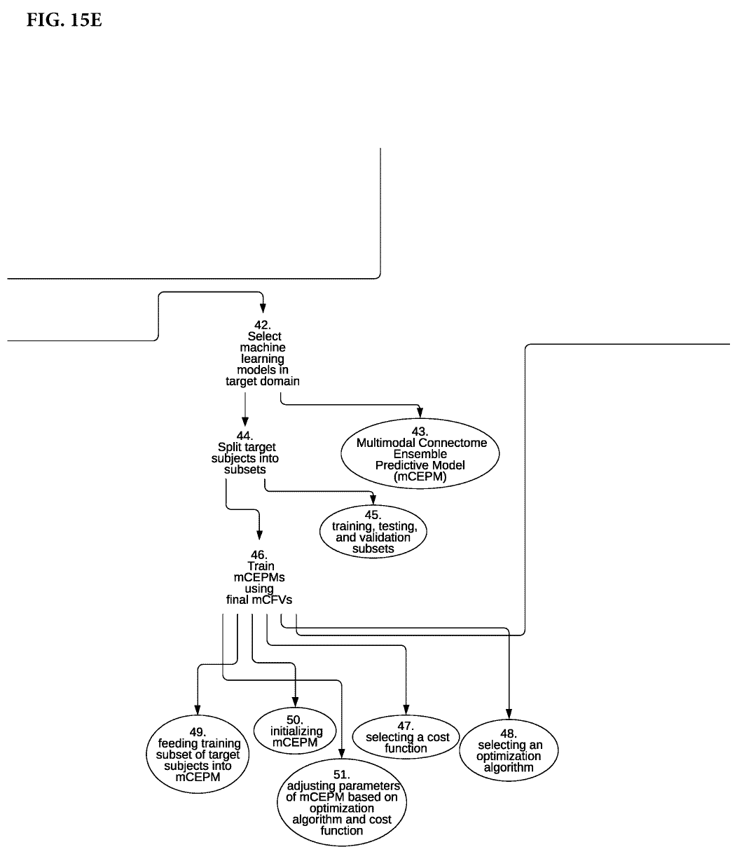

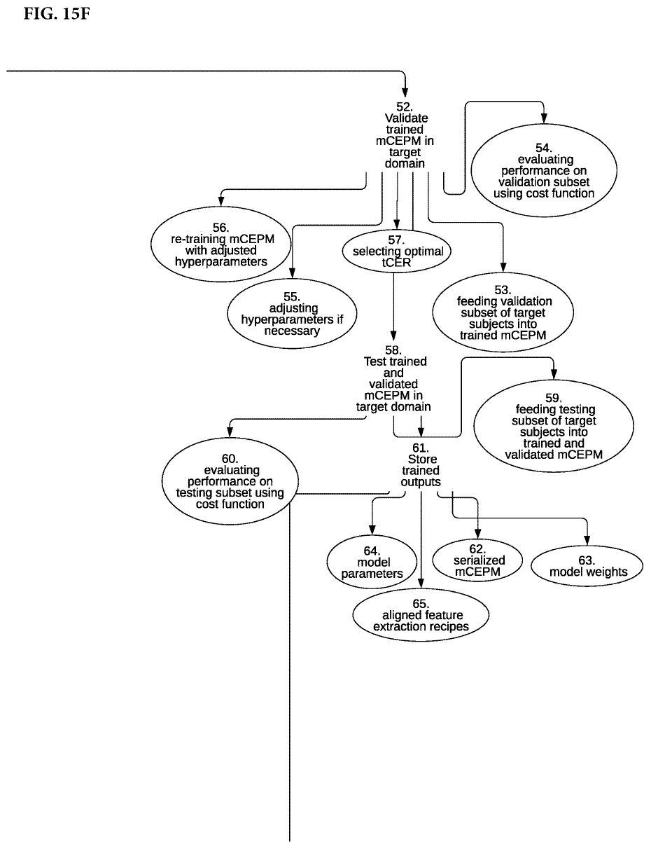

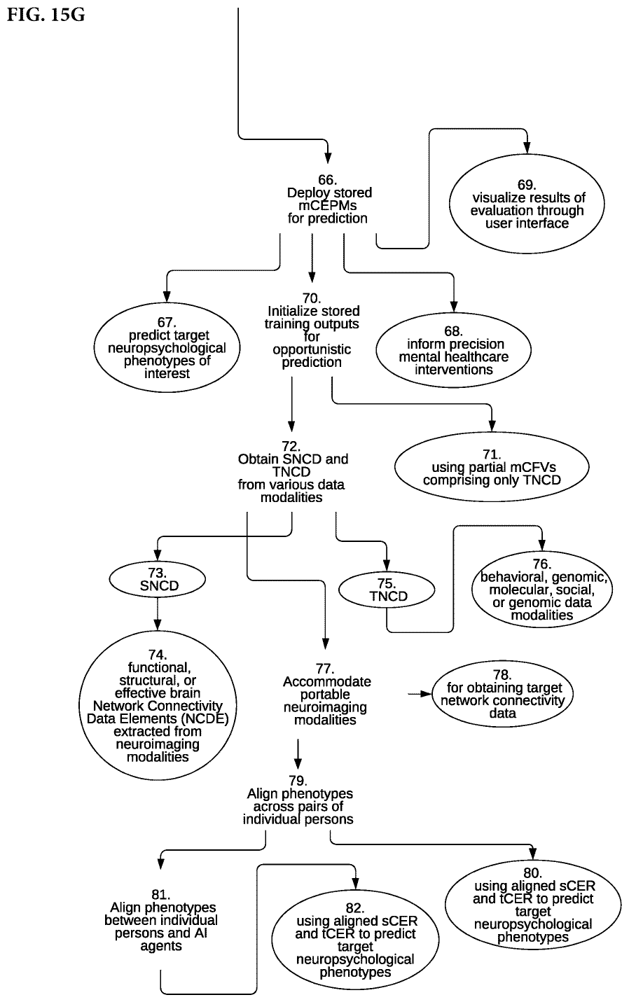

FIG. 10A to FIG. 10E are flowcharts that describe an adjacent, multimodal CETL method, according to some embodiments of the present disclosure that involves selecting one or more target domains (1 in FIG. 15C) including one or more target neuropsychological phenotypes of interest (1002 in FIG. 10A and 2 in FIG. 15C), and obtaining source network connectivity data (SNCD) (3 in FIG. 15C) from one or more pluralities of individual subjects in one or more source domains (1004 in FIG. 10A and 4 in FIG. 15C). At 1006 in FIG. 10A, the method may include selecting one or more target modalities (5 in FIG. 15B), and at 1008 in FIG. 10A, the method may include obtaining target network connectivity data (TNCD) (10 in FIG. 15C) from the selected one or more target modalities to sample data from pluralities of individual subjects in the selected one or more target domains (11-12 in FIG. 15C). In some embodiments, the obtained TNCD tracks variance in (6 in FIG. 15B) the selected target neuropsychological phenotypes (7 in FIG. 15B) and the obtained SNCD (8 in FIG. 15B), and the TNCD encodes two or more Network Connectivity Data Attributes (NCDA) (9 in FIG. 15B).

At 1010 in FIG. 10A, the method involves sampling, by a processing device, a plurality of source Connectome Graphical Models (sCGM) from the obtained SNCD for each source subject (13 in FIG. 15B). The processing device may comprise desktops or servers (14-15 in FIG. 15B), further comprising central processors, graphics processors, tensor processors, or quantum processors (16 in FIG. 15B). The sampling process may include one or more subprocesses of connectome ensemble feature engineering (17 in FIG. 15B). At 1012 in FIG. 10A, the method includes assigning two or more unique recipes of NCDA to two or more respective sCGMs (18-19 in FIG. 15B), wherein each assigned unique recipe of NCDA constitutes a sCGM view, and the sampled one or more pluralities of sCGM views make up one or more source connectome ensemble representations (sCER) (20 in FIG. 15B).

At 1014 in FIG. 10A, the method involves sampling, by a processing device, a plurality of target Connectome Graphical Models (tCGM) from the obtained TNCD for each target subject (21 in FIG. 15C). The processing device may comprise desktops or servers (22-23 in FIG. 15C), further comprising central processors, graphics processors, tensor processors, or quantum processors (24 in FIG. 15C). The sampling process may include one or more subprocesses of connectome ensemble feature engineering (25 in FIG. 15C) The sampling process may include one or more subprocesses of connectome ensemble feature engineering. At 1016 in FIG. 10B, the method includes assigning two or more unique recipes of NCDA to two or more respective tCGMs (26-27 in FIG. 15C), wherein each assigned unique recipe of NCDA constitutes a tCGM view, and the sampled one or more pluralities of tCGM views make up one or more target connectome ensemble representations (tCER) (28 in FIG. 15C).

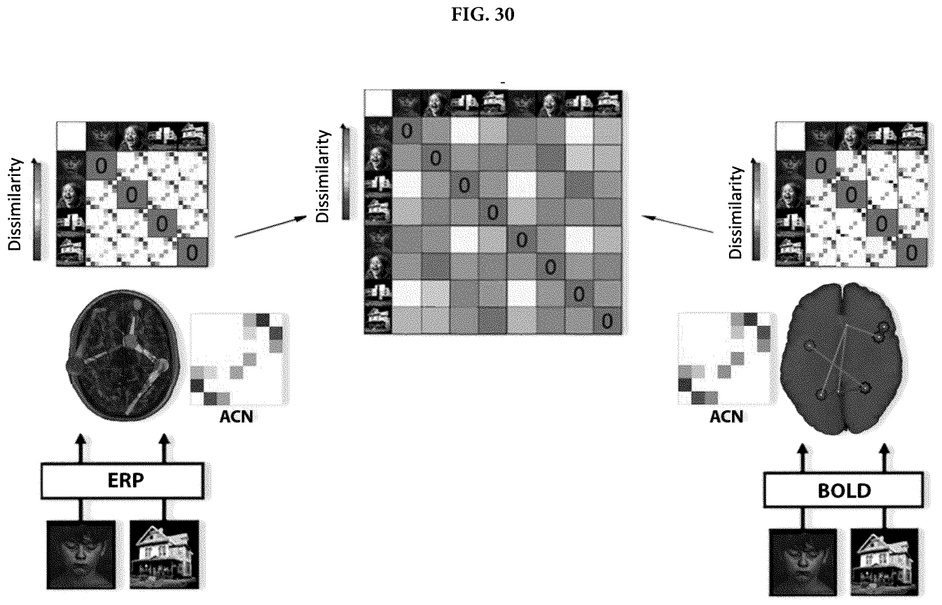

At 1018 in FIG. 10B, the method involves aligning or fusing (29 in FIG. 15D), by the processing device and through an iterative process of recursive partitioning (30 in FIG. 15D), the sampled one or more sCER with the sampled one or more tCER. The alignment or fusion process may include selecting one or more sCGM views from the one or more sCER (1020 in FIG. 10B and 31 in FIG. 15D), selecting one or more tCGM views from the one or more tCER (1022 in FIG. 10B and 31 in FIG. 15D), and selecting one or more multi-view representational alignment or fusion algorithms (1024 in FIG. 10B and 32-34 in FIG. 15D). In some embodiments, the selected one or more alignment algorithms may include distance or similarity-based alignment, such as Cross-Modal Ranking, Partial Least Squares, Cross-Modal Hashing, or Deep Cross-View Embedding Models; correlation-based alignment such as Canonical Correlation Models (CCM), Sparse CCM, Kernel CCM, or deep CCM; graphical model-based fusion such as multi-modal topic learning, multi-view sparse coding, multi-view latent space Markov networks, or multi-modal deep Boltzmann machines; Neural Network-based fusion such as multi-modal autoencoders, multi-view convolutional neural networks, multi-layer graph neural networks, hypergraph neural networks, Domain Adversarial Neural Networks, or multi-modal recurrent neural networks. The method may also involve selecting one or more alignment or fusion optimization objectives (1026 in FIG. 10B and 35 in FIG. 15D). In some embodiments, the selected one or more alignment or fusion optimization objectives may include minimizing domain discrepancy, maximizing relevance to the one or more target neuropsychological phenotypes, maximizing biological or functional significance, maximizing statistical significance, maximizing information content, maximizing contribution to explained variance, minimizing representational dissimilarity (FIG. 30), or maximizing compatibility with the one or more statistical learning models.

The method may also involve learning one or more mapping kernels (36 in FIG. 15D) that project the Network Connectivity Data Elements (NCDE) of the selected one or more sCGM views, and the NCDE of the selected one or more tCGM views, onto one or more shared or fused latent spaces (33 in FIG. 15D), respectively (1028 in FIG. 10B). The method then includes jointly embedding, by the learned one or more mapping kernels, the one or more sCGM and tCGM into one or more multimodal connectome feature vectors (mCFV) (1030 in FIG. 10C and 37 in FIG. 15D). At 1032 in FIG. 10C, the method involves selecting one or more alignment or fusion evaluation metrics (38 in FIG. 15D) with respect to the selected one or more alignment optimization objectives (38 in FIG. 15D), evaluating the quality of the alignment or fusion between the sampled one or more sCER and the sampled one or more tCER using the selected one or more alignment or fusion evaluation metrics (1034 in FIG. 10C and 39 in FIG. 15D), and adjusting the one or more mapping kernels based on the selected alignment or fusion optimization objectives (40 in FIG. 15D) and the evaluation of the alignment or fusion quality (1036 in FIG. 10C). The method concludes with obtaining a final jointly embedded one or more mCFVs (41 in FIG. 15D) after the convergence of the iterative alignment process (1038 in FIG. 10C).

The multimodal CETL method may also comprise additional steps for training, testing, and validating one or more Multimodal Connectome Ensemble Predictive Models (mCEPMs) (43 in FIG. 15E) using the final jointly embedded one or more mCFVs (45-46 in FIG. 15E). At 1040 in FIG. 10C, the method may include selecting one or more Multimodal Connectome Ensemble Predictive Models (mCEPMs) machine learning models in the target domain (42 in FIG. 15E). In some embodiments, at 1042 in FIG. 10C, the method may include splitting the obtained one or more pluralities of target subjects into training, testing, and validation subsets (44 in FIG. 15E). At 1044 in FIG. 10D, the method may include selecting a cost function (47 in FIG. 15E). At 1046 in FIG. 10D, the method may include selecting an optimization algorithm (48 in FIG. 15E). At 1048 in FIG. 10D, the method may include feeding the training subset of the obtained one or more pluralities of target subjects into the selected mCEPM (49 in FIG. 15E).

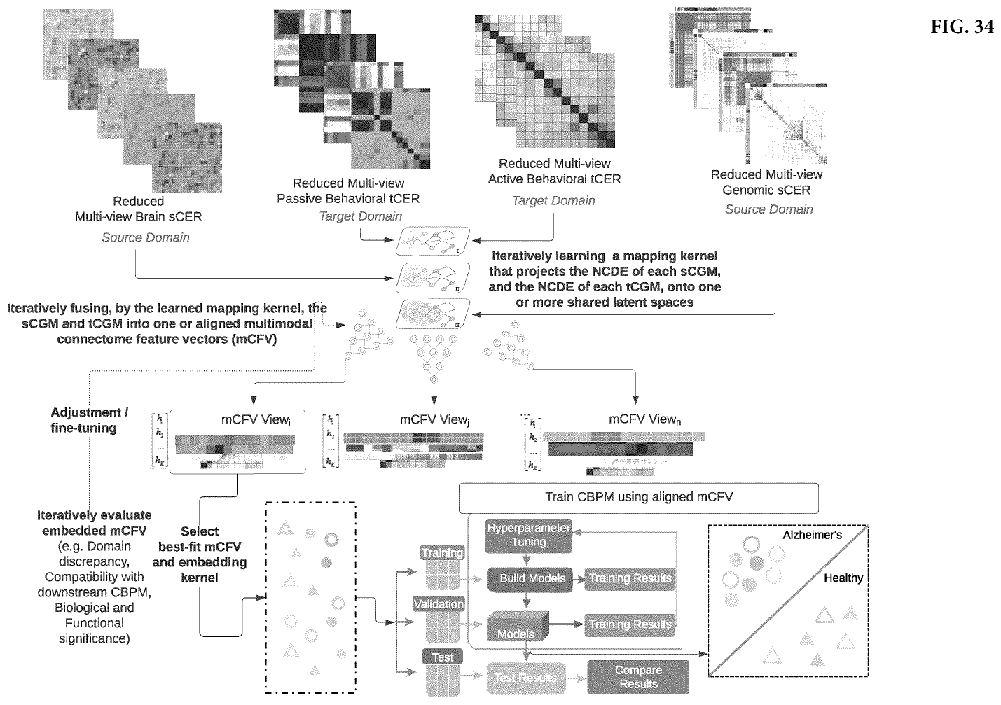

In an embodiment, the flow of data objects in this method is depicted in greater detail in the flow diagram of FIG. 34, wherein reduced (i.e. pruned) Brain sCER and genetic sCER are fused with reduced passive behavioral tCER and active behavioral tCER by the learned mapping kernel into one or more aligned multimodal connectome feature vectors (mCFV). The method then involves adjusting or fine-tuning the mCFV and the embedding kernel to optimize the alignment based on domain discrepancy, compatibility with downstream Connectome-Based Predictive Models (CBPM), biological and functional significance, and other selected optimization objectives. The best-fit mCFV and embedding kernel are then selected for further processing.

In some embodiments, at 1050 in FIG. 10D, the method may include initializing the selected mCEPM (50 in FIG. 15E). At 1052 in FIG. 10D, the method may include adjusting the parameters of the initialized mCEPM based on the selected optimization algorithm and the selected cost function (51 in FIG. 15E). At 1054 in FIG. 10D, the method may include validating, by the processing device and for each tCGM view, the trained mCEPM in the target domain (52 in FIG. 15F). At 1056 to 1066 in FIG. 10D-FIG. 10E, this involves feeding the validation subset of the obtained one or more pluralities of target subjects into the trained mCEPM (53 in FIG. 15F), evaluating the performance of the trained mCEPM on the validation subset using the selected cost function (54 in FIG. 15F), adjusting the hyperparameters of the selected mCEPM when applicable based on the performance on the validation subset (55 in FIG. 15F), re-training the selected mCEPM with the adjusted hyperparameters (56 in FIG. 15F), and selecting the one or more tCGM views with the best performance on the validation subset based on the selected cost function (57 in FIG. 15F). The validation subset may comprise the final jointly embedded one or more mCFVs of each transferred tCGM view and the corresponding one or more target neuropsychological phenotypes of interest (82 in FIG. 15G). A satisfactory performance may indicate that training performance of the mCEPM may generalize to unseen data. The adjusting may comprise a grid search, random search, or Bayesian optimization of the hyperparameters. In some embodiments, the stored outputs may be provided to a user interface or user experience that allows the user to interact with the CEPM, monitor the training, and visualize the results of the evaluation (69 in FIG. 15G). At 1068 in FIG. 10E, the method may include testing, by the processing device and for each tCGM view, the trained and validated mCEPM in the target domain (58 in FIG. 15F). This involves feeding the testing subset of the obtained one or more pluralities of target subjects into the trained and validated mCEPM (59 in FIG. 15F), and evaluating the performance of the trained and validated mCEPM on the testing subset using the selected cost function (60 in FIG. 15F). The testing subset may comprise the final jointly embedded one or more mCFVs of each transferred tCGM view and the corresponding one or more target neuropsychological phenotypes of interest (78 in FIG. 15G).

At 1068 in FIG. 10E, the method may include storing, on one or more forms of non-transitory machine-readable storage media, the one or more embedded and trained outputs (61 in FIG. 15F). The outputs may comprise the weights of the one or more trained mCEPM, the unique recipes of NCDA from the selected optimal tCER, and the selected one or more embedding algorithms (62-65 in FIG. 15F). At 1070 in FIG. 10E, the method may include deploying the stored one or more stored outputs in the target domain to predict the one or more neuropsychological phenotypes of interest (66 in FIG. 15G). In some embodiments, the user may initialize, by the processing device, the one or more stored training outputs to opportunistically predict (70 in FIG. 15G) the one or more target neuropsychological phenotypes of interest (67 in FIG. 15G). In this scenario, removing the SNCD from the aligned one or more mCFVs (71 in FIG. 15G) preserves the predictive performance of the trained one or more mCEPMs when relying only on the TNCD. The one or more target neuropsychological phenotypes of interest are predicted using only the obtained TNCD and the one or more aligned mCFVs.

In certain embodiments, the one or more modalities of SNCD (73 in FIG. 15G) may comprise structural, functional, or effective brain connectivity data extracted from one or more neuroimaging data samples (74 in FIG. 15G), or Single nucleotide polymorphism (SNP) genotype, gene co-expression profiles, Gene Regulatory Networks (GRN), or DNA methylation connectivity data extracted from one or more genomic data samples (76 in FIG. 15G). A first embodiment of the one or more modalities of TNCD (75 in FIG. 15G) may comprise behavioral, molecular, social, or portable neuroimaging data modalities (77 in FIG. 15G), such as digital connectivity data obtained from physical movement patterns, social interactions, or usage patterns of electronic devices data samples; speech, language, knowledge, or text connectivity data obtained from interviews, questionnaires, or natural language processing analysis of written or verbal communication data samples; physiological connectivity data obtained from wearable sensors, such as heart rate, galvanic skin conductance, or body temperature monitoring data samples; metabolomic or proteomic profiles obtained from blood, cerebrospinal fluid, or other molecular concentration array data samples; social network connectivity data obtained from online platforms or offline interactions data samples; or portable neuroimaging connectivity data obtained from Electroencephalography (EEG), Magnetoencephalography (MEG), Functional Near-Infrared Spectroscopy (fNIRS), or Application-Specific Integrated Circuits (ASIC). The modality-specific connectome ensemble feature engineering process is depicted for brain-based modality embodiments in FIG. 29, passive behavioral modality embodiments in FIG. 31, active behavioral modality embodiments in FIG. 32, and genetic modality embodiments in FIG. 33. In each embodiment, the raw data modality is depicted, followed by multiple further embodiments of multidimensional network connectivity data attributes, followed by sampling of respective CERs produced by decision-tree perturbation of multidimensional node and edge attributes.

Brain-based network connectivity embodiments can assume of multidimensional attributes (FIG. 29). Multidimensional node attributes can comprise: cytoarchitecture and histology, parcellation, region-of-interest, granularity, resting vs. task-evoked activity, smoothing, signal composition (electrocortical vs. hemodynamic vs. metabolic signal), signal distillation distillation, activation vs. deactivation, neuronal subtypes. Multidimensional edge attributes can comprise: structural vs. functional vs. effective, fiber type, fiber range, fiber traversal, fiber orientation, intrinsic frequency band, connectivity model, sparsity, connectivity threshold, connection strength, connection length, connection density, synchronization, information transfer, causality, effective connectivity, dynamic connectivity, topological organization, modularity, integration, segregation, network resilience, inter-hemispheric connections, intra-hemispheric connections, cross-frequency interactions.

Behavioral (Passive Measures) network connectivity embodiments can assume a variety of multidimensional attributes (FIG. 31). Multidimensional node attributes, for example, can comprise: GPS coordinates, locations visited, frequency of visits, heart-rate variability at location, weather at location, sound at location, phone usage at location, body temperature at location, date-time, entropy/time spent at location, eye gaze, saccade count, facial expressions, interactions with friends, interactions with strangers, time alone. Multidimensional edge attributes, for example, can comprise: mode of transportation, accelerometry, radius of gyration, maximum distance from home, distance travelled, travel time, physiological responses to emotions (e.g., heart rate variability, skin conductance). Behavioral (Passive Measures) network connectivity embodiments can assume a variety of multidimensional attributes (FIG. 31). Multidimensional node attributes, for example, can comprise: GPS coordinates, locations visited, frequency of visits, heart-rate variability at location, weather at location, sound at location, phone usage at location, body temperature at location, date-time, entropy/time spent at location, eye gaze, saccade count, facial expressions, interactions with friends, interactions with strangers, time alone. Multidimensional edge attributes, for example, can comprise: mode of transportation, accelerometry, radius of gyration, maximum distance from home, distance travelled, travel time, physiological responses to emotions (e.g., heart rate variability, skin conductance).

Behavioral (Active Measures) network connectivity embodiments can assume a variety of multidimensional attributes (FIG. 32). Multidimensional node attributes, for example, can comprise: reaction time, attention span, memory recall, cognitive flexibility, problem-solving skills, emotional regulation, mood fluctuations, anxiety levels, depression symptoms, sleep patterns, appetite, social interactions, motivation levels, stress response, impulsivity, aggression, empathy, self-esteem, self-awareness, language processing, pain perception, hallucinations, delusions. Multidimensional edge attributes, for example, can comprise: cognitive task performance, emotional response to stimuli, sentiment analysis in speech and writing, social behavior patterns, frequency and severity of symptoms, changes in cognitive and emotional states over time, coping mechanisms, adaptation to stressors, medication effects, therapy progress, comorbidities, genetic predisposition to cognitive, sentiment, or symptom development.

Genetic network connectivity embodiments can assume a variety of multidimensional attributes (FIG. 33). Multidimensional node attributes, for example, can comprise: gene symbols, gene identifiers, chromosome location, gene biotype, gene expression levels, gene ontology terms, molecular functions, biological processes, cellular components, protein domains, protein-protein interactions, transcription factor binding sites, epigenetic marks, single nucleotide polymorphisms (SNPs), copy number variations (CNVs), gene-disease associations, gene-drug interactions. Multidimensional edge attributes, for example, can comprise: co-expression correlation, regulatory interactions, genetic interactions, epistatic effects, protein-protein interactions, gene fusion events, shared transcription factor binding sites, shared microRNA target sites, shared pathways, shared gene ontology terms, shared gene-disease associations, shared gene-drug interactions, evolutionary conservation, genomic proximity.

The CETL method may further comprise transferring Connectome Ensemble Representations (CER) across various pairs. In one embodiment, the method involves transferring CER across one or more pairs of individual persons, wherein the tCER constitutes the CER of the other individual person in the one or more pairs of individual persons. The method includes aligning and jointly embedding the sCER and the tCER (79 in FIG. 15G), wherein the alignment and joint embedding constitute the alignment of phenotypes across the one or more pairs of individual persons. The method then predicts the one or more target neuropsychological phenotypes of interest (80 in FIG. 15G) in the target domain using the aligned phenotypes across the one or more pairs of individual persons with the multimodal connectome ensemble transfer learning method.

In another embodiment, the method involves transferring CER across one or more pairs, including an individual person and an artificially intelligent agent, wherein the tCER constitutes the CER of the artificially intelligent agent (81 in FIG. 15G). The method includes aligning and jointly embedding the sCER and the tCER, wherein the alignment and joint embedding constitute the alignment of phenotypes across the one or more pairs. The method then aligns one or more neuropsychological phenotypes (82 in FIG. 15G) from the individual person with the artificially intelligent agent in the one or more pairs using the multimodal connectome ensemble transfer learning method.

In a further embodiment, the method involves transferring CER across one or more pairs, including an individual person and an artificially intelligent agent, wherein the tCER constitutes the CER of the individual person (81 in FIG. 15G). The method includes aligning and jointly embedding the sCER and the tCER, wherein the alignment and joint embedding constitute the alignment of phenotypes across the one or more pairs. The method then aligns one or more neuropsychological phenotypes (82 in FIG. 15G) from the artificially intelligent agent with the individual person in the one or more pairs using the multimodal connectome ensemble transfer learning method.

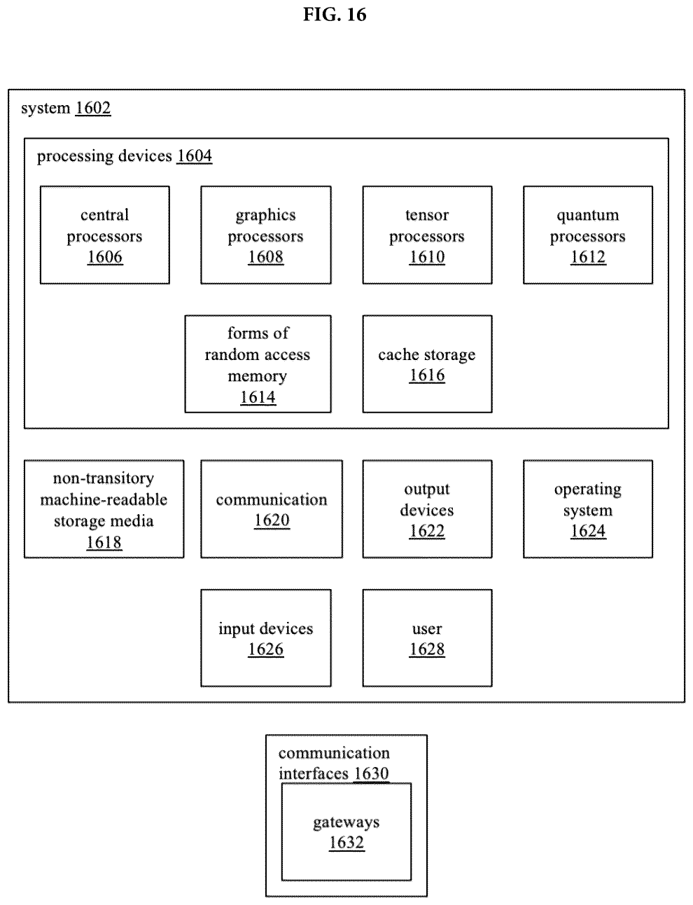

FIG. 16 is a block diagram that describes a system (1602 from FIG. 16), according to some embodiments of the present disclosure. In some embodiments, the system (1602 from FIG. 16) may include one or more communication (1620 from FIG. 16) interfaces for facilitating the transfer of data and information between the one or more processing devices (1604 from FIG. 16) and the storage media (1618 from FIG. 16). The system (1602 from FIG. 16) may also include one or more processing devices 1604 that performs automated feature engineering of connectome ensembles, one or more forms of CER pruning analysis, embeds CERs into Connectome Feature Vectors (CFVs), trains Connectome Ensemble Predictive Models (CEPMs), or transfers CERs across disparate connectivity data modalities.

In some embodiments, the system (1602 from FIG. 16) may also include one or more non-transitory machine-readable storage media 1618 for storing the obtained target and source network connectivity data, the sampled Connectome Graphical Models (CGMs), the connectome ensemble representations (CERs), the Connectome Feature Vectors (CFVs), the trained CEPMs, and their associated parameters. The system (1602 from FIG. 16) may also include one or more input devices (1626 from FIG. 16) for enabling users to interact with the system (1602 from FIG. 16) and provide input for selecting target and source domains, obtaining and preprocessing network connectivity data, and monitoring CEPM training.

In some embodiments, the system (1602 from FIG. 16) may also include one or more output devices (1622 from FIG. 16) for displaying the results of the evaluations, visualizations, and predictions generated by the CEPM, enabling users to make informed decisions about precision mental healthcare interventions. The system (1602 from FIG. 16) may also include one or more user interfaces (1628 from FIG. 16) for providing a user-friendly environment to access and interact with the system 1602, input data, configure settings, and view the results. The system (1602 from FIG. 16) may also include an operating system (1624 from FIG. 16) configured to manage the system resources, execute the processes, and coordinate the activities of the one or more processing devices (1604 from FIG. 16), communication interfaces (1630 from FIG. 16), input devices, output devices, and user interfaces.

In some embodiments the system (1602 from FIG. 16) may include one or more processing devices (1604 from FIG. 16) that include central processors (1606 from FIG. 16), graphics processors (1608 from FIG. 16), tensor processors (1610 from FIG. 16), quantum processors (1612 from FIG. 16), one or more forms of random access memory (1614 from FIG. 16), and cache storage (1616 from FIG. 16). The one or more processing devices (1604 from FIG. 16) may be configured using a distributed or parallel computing architecture that initialize and execute Directed Acyclic Graphs, and facilitating efficient processing of large-scale, high-dimensional, or multi-modal network connectivity and phenotypic data.

In some embodiments the system (1602 from FIG. 16) may include one or more operating systems may be configured to adapt its processes and workflows dynamically based on available computational resources, data quality, and user-defined constraints or requirements. The one or more communication interfaces (1630 from FIG. 16) may also include one or more gateways (1632 from FIG. 16) configured to receive and process data from a wide variety of network connectivity data modalities, data formats, and hardware configurations.

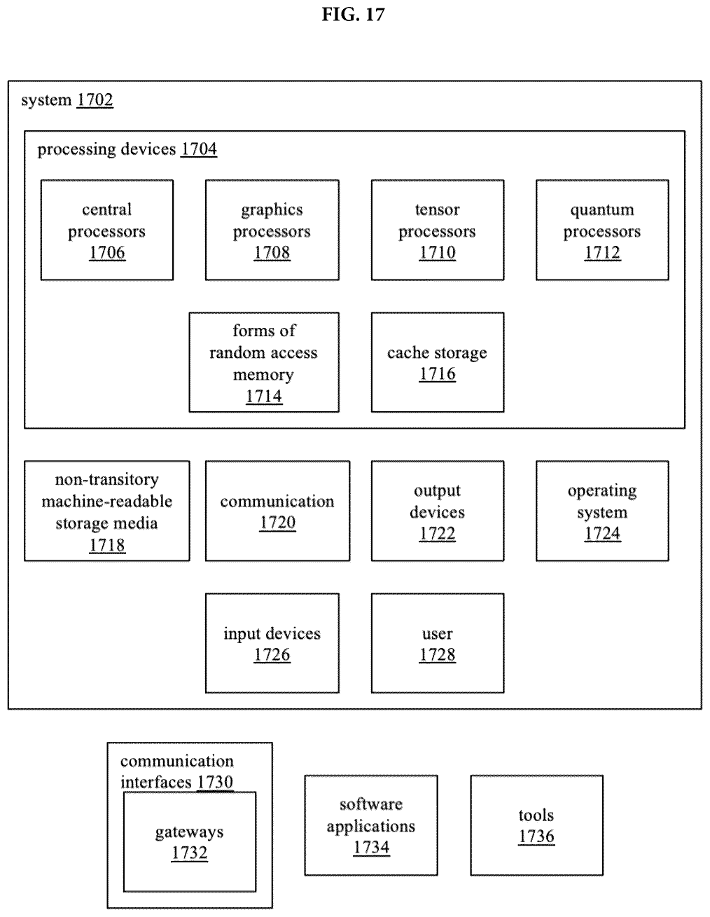

In some embodiments the system (1702 from FIG. 17) may include one or more communication interfaces (1730 from FIG. 17) may be connected to or integrated with one or one or more electronic health record systems, clinical decision support systems, or health information exchange (HIE) platforms for seamless data exchange, interoperability, and real-time deployment of the CEPMs in clinical settings. In some embodiments, an embodiment of the system (1702 from FIG. 17) of synchronized computer hardware. In some embodiments, an embodiment of the one or more output devices.

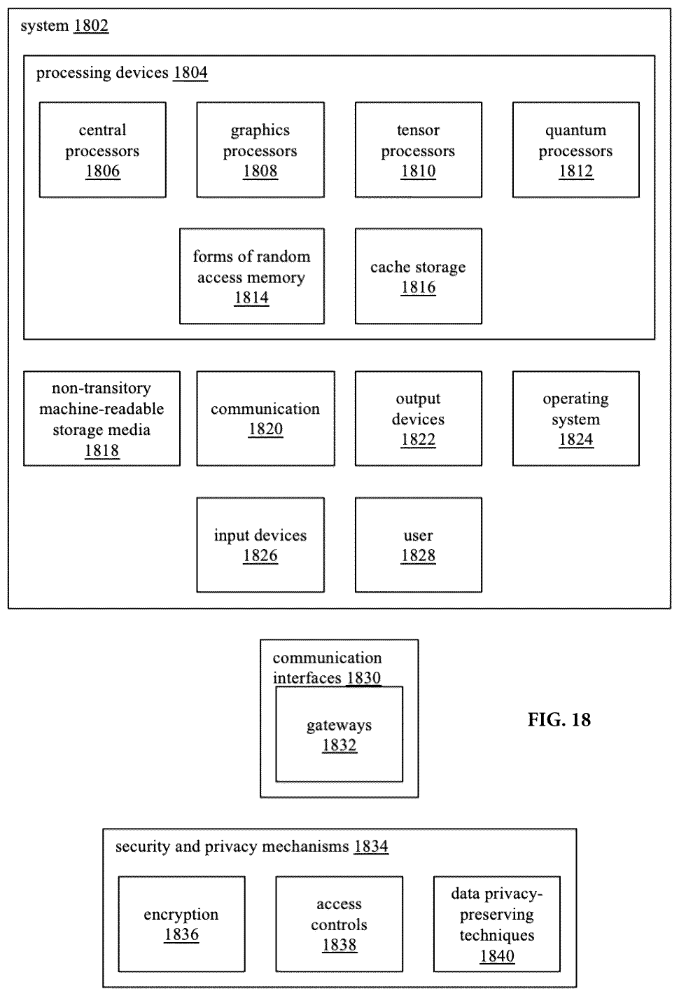

FIG. 18 is a block diagram that further describes the system (1802 from FIG. 18), according to some embodiments of the present disclosure. Similarly, FIG. 18 from FIG. 18 is a block diagram that further describes the system 1802 from FIG. 18, according to some embodiments of the present disclosure. In some embodiments, the security and privacy mechanisms (1834 from FIG. 18) may include encryption (1836 from FIG. 18), access controls (1838 from FIG. 18), and data privacy-preserving techniques (1840 from FIG. 18).

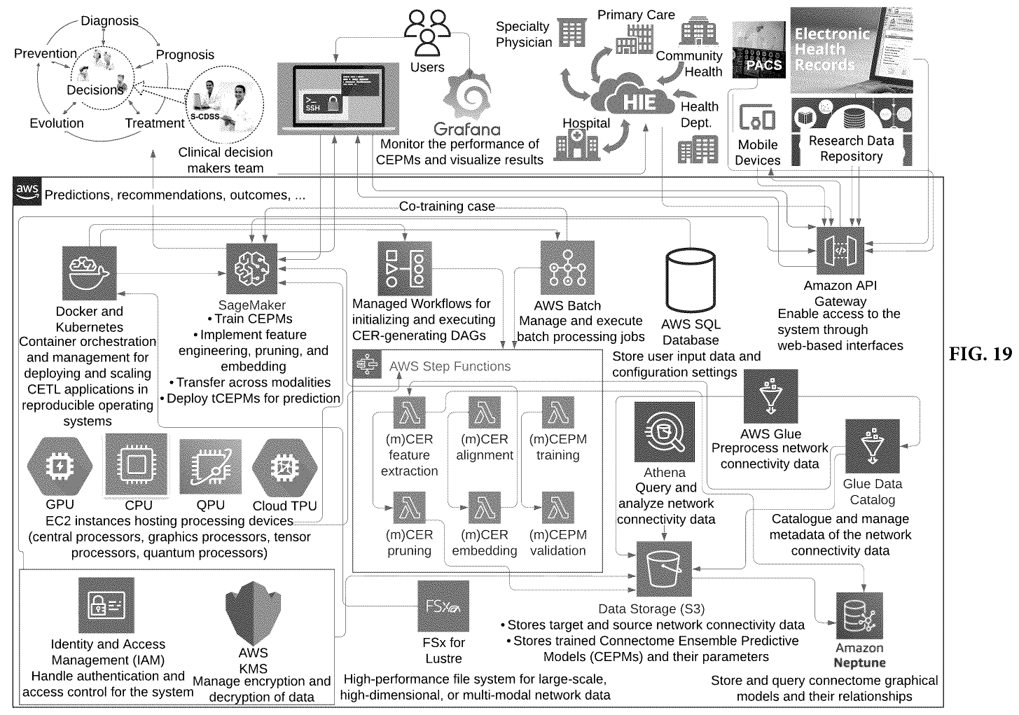

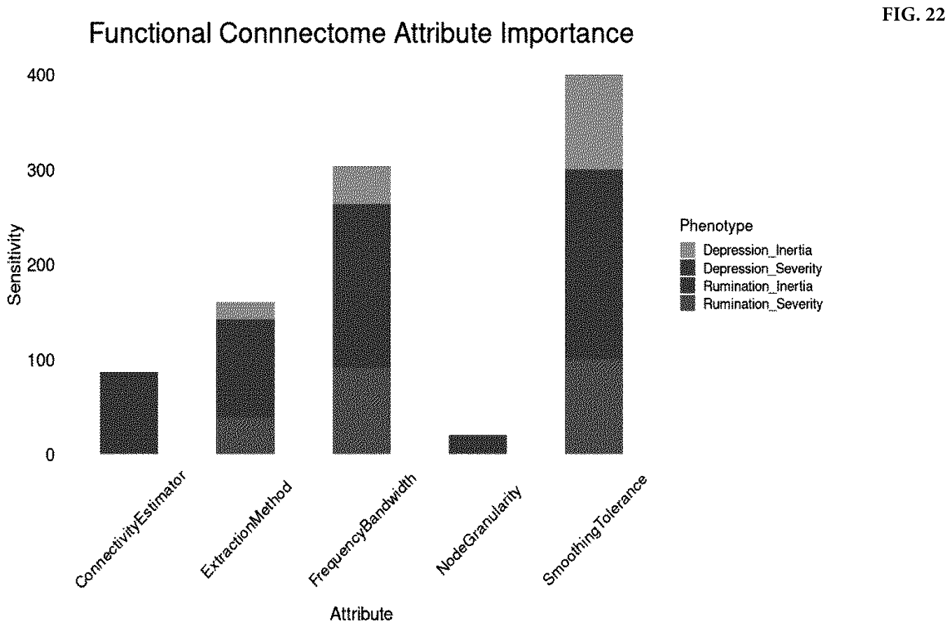

FIG. 19-FIG. 23 are example embodiments demonstrating the dimensionality reduction step of brain-based connectome ensemble transfer learning, whereby a large set of sampled source CER are iteratively reduced by eliminating unreliable and indiscriminable connectome views, and selecting and/or aggregating further subsets of the ensuing discriminable recipes that co-vary with candidate maintaining mechanisms of chronic depression (e.g. rumination, executive dysfunction, anhedonia) and at various temporal resolutions (i.e. severity and persistence). Rumination persistence might be simply defined on the basis of shared variance in rumination severity across multiple longitudinal time-points, for instance. Since it remains unclear whether anhedonia is a stable construct over time in depressed patients (trait) or a symptom that fluctuates depending on severity or even antidepressant mechanisms (state), however, more sophisticated latent variable models (particularly longitudinal Structural Equation Models (SEM) FIG. 22) that are iteratively trained on CGM features might be used to select subsets of CGM attributions from one or more candidate CGM views. Regardless of whether classification, regression, or SEM are used, biased manual tuning and/or model fitting performed differentially across different CGM attributions poses a grave threat to transfer learning. Namely, it confounds the researcher's ability to precisely gauge the predictive value of a feature extraction recipe of CGM attribution in a true, unbiased multiverse analysis (FIG. 21). In addition to homogeneous handing of CEPM fitting where a secondary phenotype is used as the outcome variable, it is similarly important to control the number of feature inputs to avoid introducing biases due to inconsistent feature dimensionality across the DAG. To mitigate predictive variance at this stage, it may further be advantageous to learn CGM views following a multi-task learning framework that deliberately optimizes for heterogeneous variance, such as across multiple etiological mechanisms when the target domain is diagnosis, or across one or more maintaining mechanisms spanning multiple time-intervals when the target domain involves prognosis.

Citations are Herein Incorporated by Reference

### REFERENCES

[1] Agnar Aamodt and Enric Plaza. “Case-Based Reasoning: Foundational Issues, Methodological Variations, and System Approaches”. In: Ai Commun. 7.1 (1994), pp. 39-59. ISSN: 0921-7126. DOI: 10.3233/aic-1994-7104.
[2] Tuomas Alakorkko et al. “Effects of spatial smoothing on functional brain networks”. In: Eur J Neurosci 46.9 (2017), pp. 2471-2480. ISSN: 0953-816X. DOI: 10.1111/ejn.13717.
[3] Zaniar Ardalan and Vignesh Subbian. “Transfer Learning Approaches for Neuroimaging Analysis: A Scoping Review”. In: Frontiers in Artificial Intelligence 5 (February 2022). DOI: 10.3389/frai.2022.780405. URL: https://doi.org/10.3389/frai.2022.780405.
[4] Peter W. Battaglia et al. “Relational Inductive Biases, Deep Learning, and Graph Networks”. In: arXiv preprint arXiv:1806.01261 (2018), pp. 1-40.
[5] Robert Becker and Alexis Hervais-Adelman. “Resolving the Connectome, Spectrally-Specific Functional Connectivity Networks and Their Distinct Contributions to Behavior”. In: eNeuro 7.5 (2020), ENEURO.0101-20.2020. ISSN: 2373-2822. DOI: 10.1523/eneuro.0101-20.2020.
[6] Shai Ben-David et al. “A theory of learning from different domains”. In: Machine Learning 79.1-2 (October 2009), pp. 151-175. DOI: 10.1007/s10994-009-5152-4. URL: https://doi.org/10.1007/s10994-009-5152-4.
[7] Richard F. Betzel et al. “Optimally controlling the human connectome: The role of network topology”. In: Sci Rep 6.1 (2016), p. 30770. ISSN: 2045-2322. DOI: 10.1038/srep30770.
[8] Janine Bijsterbosch et al. “Challenges and future directions for representations of functional brain organization”. In: Nat Neurosci 23.12 (2020), pp. 1484-1495. ISSN: 1097-6256, 1546-1726. DOI: 10.1038/s41593-020-00726-z.
[9] Leonardo Bonilha et al. “Reproducibility of the Structural Brain Connectome Derived from Diffusion Tensor Imaging”. In: PLoS ONE 10.9 (2015), e0135247. ISSN: 1932-6203. DOI: 10.1371/journal.pone.0135247.
[10] Rotem Botvinik-Nezer et al. “fMRI data of mixed gambles from the Neuroimaging Analysis Replication and Prediction Study”. In: Sci Data 6.1 (2019), pp. 84-88. ISSN: 2052-4463. DOI: 10.1038/s41597-019-0113-7.
[11] S Bozinovski and A Fulgosi. “The influence of pattern similarity and transfer learning upon training of a base perceptron b2”. In: Proceedings of Symposium Informatica. 1976, pp. 3-121.
[12] Felix Brandl et al. “The Role of Brain Connectome Imaging in the Estimation of Depressive Relapse Risk”. In: Fortschr Röntgenstr 190.11 (2018), pp. 1036-1043. ISSN: 1438-9029, 1438-9010. DOI: 10.1055/a-0628-7260.
[13] Eric W. Bridgeford et al. “Optimal Experimental Design for Big Data: Applications in Brain Imaging”. In: bioRxiv (2019), pp. 1-18. DOI: 10.1101/802629.
[14] Joel Buishas, Ian G. Gould, and Andreas A. Linninger. “A computational model of cerebrospinal fluid production and reabsorption driven by Starling forces”. In: Croatian Medical Journal 55.5 (October 2014), pp. 481-497. DOI: 10.3325/cmj.2014.55.481. URL: https://doi.org/10.3325/cmj.2014.55.481.
[15] Cesar F. Caiafa and Franco Pestilli. “Multidimensional encoding of brain connectomes”. In: Sci Rep 7.1 (2017). ISSN: 2045-2322. DOI: 10.1038/s41598-017-09250-w.
[16] F. Campolongo, A. Saltelli, and S. Tarantola. “Sensitivity Analysis as an Ingredient of Modeling”. In: Statist. Sci. 15.4 (2000), pp. 377-395. ISSN: 0883-4237. DOI: 10.1214/ss/1009213004.
[17] Jaime G Carbonell. “Learning by analogy: Formulating and generalizing plans from past experience”. In: Machine Learning: An Artificial Intelligence Approach. Ed. by Ryszard S. Michalski, Jaime G. Carbonell, and Tom M. Mitchell. Springer Berlin Heidelberg, 1983, pp. 137-161. DOI: 10.1007/978-3-662-12405-5_5.
[18] Xiao Chen et al. “The subsystem mechanism of default mode network underlying rumination: A reproducible neuroimaging study”. In: Neuroimage 221 (2020), p. 117185. ISSN: 1053-8119. DOI: 10.1016/j.neuroimage.2020.117185.
[19] Zhe Sage Chen et al. “Modern views of machine learning for precision psychiatry”. In: Patterns 3.11 (November 2022), p. 100602. DOI: 10.1016/j.patter.2022.100602. URL: https://doi.org/10.1016/j.patter. 2022.100602.
[20] Allegra Conti et al. “Variability and Reproducibility of Directed and Undirected Functional MRI Connectomes in the Human Brain”. In: Entropy-switz. 21.7 (2019), p. 661. ISSN: 1099-4300. DOI: 10.3390/e21070661.
[21] Marc-Alexandre COte et al. “Tractometer: Towards validation of tractography pipelines”. In: Med. Image Anal. 17.7 (2013), pp. 844-857. ISSN: 1361-8415. DOI: 10.1016/j.media. 2013.03.009.
[22] R. Cameron Craddock et al. “A whole brain fMRI atlas generated via spatially constrained spectral clustering”. In: Hum. Brain Mapp. 33.8 (2011), pp. 1914-1928. ISSN: 1065-9471. DOI: 10.1002/hbm.21333.
[23] Marco Cremonini and Francesca Casamassima. “Controllability of social networks and the strategic use of random information”. In: Computational social networks 4.1 (2017), pp. 1-22.
[24] Kamalaker Dadi et al. “Benchmarking Functional Connectome-Based Predictive Models for Resting-State fMRI.” In: Neuroimage (2018).
[25] Kamalaker Dadi et al. “Fine-grain atlases of functional modes for fMRI analysis”. In: Neuroimage 221 (2020), p. 117126. ISSN: 1053-8119. DOI: 10.1016/j.neuroimage. 2020.117126.
[26] Jessica Dafflon et al. “A guided multiverse study of neuroimaging analyses”. In: Nature Communications 13.1 (June 2022). DOI: 10.1038/s41467-022-31347-8. URL: https://doi.org/10.1038/s41467-022-31347-8.
[27] Bryce S DeWitt. The Many Worlds Interpretation of Quantum Mechanics. Princeton University Press, 2015. ISBN: 9781400868056. DOI: 10.1515/9781400868056.
[28] M. Drakesmith et al. “Overcoming the effects of false positives and threshold bias in graph theoretical analyses of neuroimaging data”. In: Neuroimage 118 (2015), pp. 313-333. ISSN: 1053-8119. DOI: 10.1016/j.neuroimage. 2015.05.011.
[29] Henry C Ellis. “The transfer of learning.” In: Journal of Experimental Psychology (1964).
[30] Emily S Finn et al. “Functional connectome fingerprinting: Identifying individuals using patterns of brain connectivity”. In: Nat Neurosci 18.11 (2015), pp. 1664-1671. ISSN: 1097-6256, 1546-1726. DOI: 10.1038/nn.4135.
[31] Emily S. Finn et al. “Can brain state be manipulated to emphasize individual differences in functional connectivity?” In: Neuroimage 160 (2017), pp. 140-151. ISSN: 1053-8119. DOI: 10.1016/j.neuroimage. 2017.03.064.
[32] Eloy Garcia-Cabello et al. “The Cognitive Connectome in Healthy Aging”. In: Frontiers in Aging Neuroscience 13 (August 2021). DOI: 10.3389/fnagi. 2021.694254. URL: https://doi.org/10.3389/fnagi.2021.694254.
[33] Cedric E. Ginestet et al. “Brain Network Analysis: Separating Cost from Topology Using Cost-Integration”. In: PLoS ONE 6.7 (2011), e21570. ISSN: 1932-6203. DOI: 10.1371/journal.pone. 0021570.
[34] Arthur Gretton et al. “A Kernel Two-Sample Test”. In: 13 (2012), pp. 723-773.
[35] P Hagmann et al. “Diffusion Spectrum Imaging Tractography in Complex Cerebral White Matter: An Investigation of the Centrum Semiovale”. In: Biology. International Society for Magnetic Resonance in Medicine (2004), p. 623.
[36] Trevor Hastie, Robert Tibshirani, and Jerome Friedman. “The Elements of Statistical Learning”. In: Elements 1 (2009), pp. 337-387. DOI: 10.1007/b94608.
[37] Adrian Hernandez and Jose M. Amiga. “Attention Mechanisms and Their Applications to Complex Systems”. In: Entropy 23.3 (February 2021), p. 283. DOI: 10.3390/e23030283. URL: https://doi.org/10.3390/e23030283.
[38] Mahan Hosseini et al. “I tried a bunch of things: The dangers of unexpected overfitting in classification of brain data”. In: Neuroscience & Biobehavioral Reviews 119 (2020), pp. 456-467. ISSN: 0149-7634. DOI: 10.1016/j.neubiorev.2020.09.036.
[39] Carbonell J. G. “Toward a Theory of Knowledge Reuse: Types of Knowledge Reuse Situations and Factors in Reuse Success”. In: J. Manage. Inform. Syst. 18.1 (2001), pp. 57-93. ISSN: 0742-1222, 1557-928X. DOI: 10.1080/07421222.2001.11045671.
[40] Iain G. Johnston et al. “Precision identification of high-risk phenotypes and progression pathways in severe malaria without requiring longitudinal data”. In: npj Digit. Med. 2.1 (2019). ISSN: 2398-6352. DOI: 10.1038/s41746-019-0140-y.
[41] Daniel Kahneman, Paul Slovic, and Amos Tversky, eds. Judgment under Uncertainty, Heuristics and Biases. Cambridge University Press, 1982. DOI: 10.1017/cbo9780511809477.
[42] Myunghwan Kim and Jure Leskovec. Multiplicative Attribute Graph Model of Real-World Networks. 2010. DOI: 10.1007/978-3-642-18009-5\\_7.
[43] Nicolas Langer, Andreas Pedroni, and Lutz Jäncke. “The Problem of Thresholding in Small-World Network Analysis”. In: PLoS ONE 8.1 (2013), e53199. ISSN: 1932-6203. DOI: 10.1371/journal.pone.0053199.
[44] Etai Littwin and Lior Wolf. Complexity of multiverse networks and their multilayer generalization. 2016. DOI: 10.1109/icpr.2016.7899662.
[45] Etai Littwin and Lior Wolf. “The Multiverse Loss for Robust Transfer Learning”. In: 2016 IEEE Conference on Computer Vision and Pattern Recognition (CVPR) (2016), pp. 3957-3966.
[46] Xinyi Liu et al. “Alterations of core structural network connectome associated with suicidal ideation in major depressive disorder patients”. In: Transl Psychiatry 11.1 (2021). ISSN: 2158-3188. DOI: 10.1038/s41398-021-01353-3.
[47] Hehuan Ma et al. “Multi-view graph neural networks for molecular property prediction”. In: arXiv preprint arXiv:2005.13607 (2020).
[48] Nikos Makris et al. “Segmentation of Subcomponents within the Superior Longitudinal Fascicle in Humans: A Quantitative, In Vivo, DT-MRI Study”. In: Cereb. Cortex 15.6 (2004), pp. 854-869. ISSN: 1460-2199, 1047-3211. DOI: 10.1093/cercor/bhh186.
[49] Itzik Malkiel and Lior Wolf. “Maximal Multiverse Learning for Promoting Cross-Task Generalization of Fine-Tuned Language Models”. In: Proceedings of the 16th Conference of the European Chapter of the Association for Computational Linguistics: Main Volume. Association for Computational Linguistics, 2021. DOI: 10.18653/v1/2021.eacl-main.14.
[50] Sreevalsan S. Menon and K. Krishnamurthy. “Multimodal Ensemble Deep Learning to Predict Disruptive Behavior Disorders in Children”. In: Frontiers in Neuroinformatics 15 (November 2021). DOI: 10.3389/fninf.2021.742807. URL: https://doi.org/10.3389/fninf.2021.742807.
[51] Eirini Messaritaki, Stavros I. Dimitriadis, and Derek K. Jones. “Optimization of graph construction can significantly increase the power of structural brain network studies”. In: Neuroimage 199 (April 2019), pp. 495-511. ISSN: 1053-8119. DOI: 10.1016/j.neuroimage.2019.05.052.
[52] Lisa D. Nickerson. “Replication of Resting State-Task Network Correspondence and Novel Findings on Brain Network Activation During Task fMRI in the Human Connectome Project Study”. In: Sci Rep 8.1 (2018). ISSN: 2045-2322. DOI: 10.1038/s41598-018-35209-6.
[53] Aki Nikolaidis et al. “Suboptimal phenotypic reliability impedes reproducible human neuroscience”. In: bioRxiv (2022).
[54] Gherman Novakovsky et al. “Biologically relevant transfer learning improves transcription factor binding prediction”. In: Genome Biol 22.1 (2021). ISSN: 1474-760X. DOI: 10.1186/s13059-021-02499-5.
[55] David O'Connor et al. “Resample aggregating improves the generalizability of connectome predictive modeling”. In: Neuroimage 236 (2021), p. 118044. ISSN: 1053-8119. DOI: 10.1016/j.neuroimage.2021.118044.
[56] Stuart Oldham et al. “The efficacy of different preprocessing steps in reducing motion-related confounds in diffusion MRI connectomics”. In: Neurolmage 222 (2020), p. 117252. DOI: 10.1016/j.neuroimage.2020.117252.
[57] D. Opitz and R. Maclin. “Popular Ensemble Methods: An Empirical Study”. In: jair 11 (1999), pp. 169-198. ISSN: 1076-9757. DOI: 10.1613/jair.614.
[58] Shraddha Pai and Gary D. Bader. “Patient Similarity Networks for Precision Medicine”. In: J. Mol. Biol. 430.18 (2018), pp. 2924-2938. ISSN: 0022-2836. DOI: 10.1016/j.jmb.2018.05.037.
[59] Usama Pervaiz et al. “Optimising network modelling methods for fMRI”. In: Neuroimage 211 (2020), p. 116604. ISSN: 1053-8119. DOI: 10.1016/j.neuroimage.2020.116604.
[60] Steven M Peterson et al. “Generalized neural decoders for transfer learning across participants and recording modalities”. In: J. Neural Eng. 18.2 (2021), p. 026014. ISSN: 1741-2560, 1741-2552. DOI: 10.1088/1741-2552/abda0b.
[61] Lorien Y Pratt. “Discriminability-Based Transfer between Neural Networks”. In: Adv Neural Inf Process Syst (1993), pp. 204-211.
[62] Pranjal Ranjan, Sarvesh Patil, and Faruk Kazi. “Improved Generalizability of Deep-Fakes Detection using Transfer Learning Based CNN Framework”. In: 2020 3rd International Conference on Information and Computer Technologies (ICICT). IEEE, 2020, pp. 86-90. DOI: 10.1109/icict50521.2020.00021.
[63] Jonas Richiardi et al. “Correlated gene expression supports synchronous activity in brain networks”. In: Science 348.6240 (June 2015), pp. 1241-1244. DOI: 10.1126/science.1255905. URL: https://doi org/10.1126/science.1255905.
[64] Raiff Rodriguez-Cruces, Boris C. Bernhardt, and Luis Concha. “Multidimensional associations between cognition and connectome organization in temporal lobe epilepsy”. In: Neuroimage 213 (2020), p. 116706. ISSN: 1053-8119. DOI: 10.1016/j.neuroimage.2020.116706.
[65] Ingrid A. C. Romme et al. “Connectome Disconnectivity and Cortical Gene Expression in Patients With Schizophrenia”. In: Biological Psychiatry 81.6 (March 2017), pp. 495-502. DOI: 10.1016/j.biopsych.2016.07.012. URL: https://doi.org/10.1016/j.biopsych.2016.07.012.
[66] James K. Ruffle et al. “The autonomic brain: Multi-dimensional generative hierarchical modelling of the autonomic connectome”. In: Cortex 143 (October 2021), pp. 164-179. DOI: 10.1016/j.cortex.2021.06.012. URL: https://doi.org/10.1016/j.cortex.2021.06.012.
[67] A. Saltelli, S. Tarantola, and K. P.-S. Chan. “A Quantitative Model-Independent Method for Global Sensitivity Analysis of Model Output”. In: Technometrics 41.1 (1999), pp. 39-56. ISSN: 0040-1706, 1537-2723. DOI: 10.1080/00401706.1999.10485594.
[68] Aravind Sankar et al. “DySAT”. In: Proceedings of the 13th International Conference on Web Search and Data Mining. ACM, January 2020. DOI: 10.1145/3336191.3371845. URL:https://doi.org/10.1145/3336191.3371845.
[69] Tabinda Sarwar, Kotagiri Ramamohanarao, and Andrew Zalesky. “Mapping
connectomes with diffusion MRI: Deterministic or probabilistic tractography?” In: Magn Reson Med 81.2 (2018), pp. 1368-1384. ISSN: 0740-3194, 1522-2594. DOI: 10.1002/mrm.27471.
[70] Artur Schumacher-Schuh et al. “Advances in Proteomic and Metabolomic Profiling of Neurodegenerative Diseases”. In: Frontiers in Neurology 12 (January 2022). DOI: 10.3389/fneur.2021.792227. URL: https://doi.org/10.3389/fneur.2021.792227.
[71] B. Seijo-Pardo et al. “Ensemble feature selection: Homogeneous and heterogeneous approaches”. In: Knowl-based. Syst. 118 (2017), pp. 124-139. ISSN: 0950-7051. DOI: 10.1016/j.knosys.2016.11.017.
[72] Anthony Sicilia, Xingchen Zhao, and Seong Jae Hwang. “Domain adversarial neural networks for domain generalization: when it works and how to improve”. In: Machine Learning (April 2023). DOI: 10.1007/s10994-023-06324-x. URL: https://doi.org/10.1007/s10994-023-06324-x.
[73] Joseph P. Simmons, Leif D. Nelson, and Uri Simonsohn. “False-Positive Psychology. Undisclosed Flexibility in Data Collection and Analysis Allows Presenting Anything as Significant”. In: Psychol Sci 22.11 (2011), pp. 1359-1366. ISSN: 0956-7976, 1467-9280. DOI: 10.1177/0956797611417632.
[74] Michel R. T. Sinke et al. “Diffusion MRI-based cortical connectome reconstruction: Dependency on tractography procedures and neuroanatomical characteristics”. In: Brain Struct Funct 223.5 (2018), pp. 2269-2285. ISSN: 1863-2653, 1863-2661. DOI: 10.1007/s00429-018-1628-y.
[75] Olaf Sporns and Jonathan D. Zwi. “The Small World of the Cerebral Cortex”. In: NI 2.2 (2004), pp. 145-162. ISSN: 1539-2791. DOI: 10.1385/n1:2:2:145.
[76] Sara Steegen et al. “Increasing Transparency Through a Multiverse Analysis”. In: Perspect Psychol Sci 11.5 (2016), pp. 702-712. ISSN: 1745-6916, 1745-6924. DOI: 10.1177/1745691616658637.
[77] Shiliang Sun, Honglei Shi, and Yuanbin Wu. “A survey of multi-source domain adaptation”. In: Inform. Fusion 24 (2015), pp. 84-92. ISSN: 1566-2535. DOI: 10.1016/j.inffus.2014.12.003.
[78] Hiromasa Takemura et al. “Ensemble Tractography”. In: PLoS Comput Biol 12.2 (2016), e1004692. ISSN: 1553-7358. DOI: 10.1371/journal.pcbi.1004692.
[79] Eric Tzeng et al. “Adversarial discriminative domain adaptation”. In: Proceedings of the IEEE conference on computer vision and pattern recognition. 2017, pp. 7167-7176.
[80] Mohammed Uddin, Yujiang Wang, and Marc Woodbury-Smith. “Artificial intelligence for precision medicine in neurodevelopmental disorders”. In: npj Digit. Med. 2.1 (2019). ISSN: 2398-6352. DOI: 10.1038/s41746-019-0191-0.
[81] Gaël Varoquaux and R. Cameron Craddock. “Learning and comparing functional connectomes across subjects”. In: Neuroimage 80 (2013), pp. 405-415. ISSN: 1053-8119. DOI: 10.1016/j.neuroimage.2013.04.007.
[82] Gaël Varoquaux et al. “Atlases of cognition with large-scale human brain mapping”. In: PLoS Comput Biol 14.11 (October 2018), e1006565. ISSN: 1553-7358. DOI: 10.1371/journal.pcbi.1006565.
[83] Petar Veliekovie et al. “Graph Attention Networks”. In: International Conference on Learning Representations (2018). URL: https://openreyiew.net/forum?id=rJXMpikCZ.
[84] Joel Veness et al. “Context Tree Switching”. In: 2012 Data Compression Conference. Ed. by H. Larochelle et al. Vol. 33. IEEE, 2012, pp. 918-929. DOI: 10.1109/dcc.2012.39.
[85] Diego Vidaurre, Stephen M. Smith, and Mark W. Woolrich. “Brain network dynamics are hierarchically organized in time”. In: Proc Natl Acad Sci USA 114.48 (2017), pp. 12827-12832. ISSN: 0027-8424, 1091-6490. DOI: 10.1073/pnas.1705120114.
[86] Theo Vos et al. “The Burden of Major Depression Avoidable by Longer-term Treatment Strategies”. In: Arch Gen Psychiatry 61.11 (2004), p. 1097. ISSN: 0003-990X. DOI: 10.1001/archpsyc.61.11.1097.
[87] Jin-Hui Wang et al. “Graph Theoretical Analysis of Functional Brain Networks: Test-retest Evaluation on Short- and Long-Term Resting-State Functional MRI Data”. In: PLoS ONE 6.7 (2011), e21976. ISSN: 1932-6203. DOI: 10.1371/journal.pone.0021976.
[88] Jinhui Wang et al. “GRETNA: A graph theoretical network analysis toolbox for imaging connectomics”. In: Front. Hum. Neurosci. 9 (2015), pp. 1-16. ISSN: 1662-5161. DOI: 10.3389/fnhum.2015.00386.
[89] Zhihao Wang et al. “Connectome-Based Predictive Modeling of Individual Anxiety”. In: Cereb. Cortex 31.6 (2021), pp. 3006-3020. ISSN: 1047-3211, 1460-2199. DOI: 10.1093/cercor/bhaa407.
[90] Bernadette C. M. van Wijk, Cornelis J. Stam, and Andreas Daffertshofer. “Comparing Brain Networks of Different Size and Connectivity Density Using Graph Theory”. In: PLoS ONE 5.10 (2010), e13701. ISSN: 1932-6203. DOI: 10.1371/journal.pone.0013701.
[91] R. S. Woodworth and E. L. Thorndike. “The influence of improvement in one mental function upon the efficiency of other functions. (I).” In: Psychol. Rev. 8.3 (1901), pp. 247-261. ISSN: 1939-1471, 0033-295X. DOI: 10.1037/h0074898.
[92] Cedric Huchuan Xia et al. “Mobile footprinting: linking individual distinctiveness in mobility patterns to mood, sleep, and brain functional connectivity”. In: Neuropsychopharmacology 47.9 (June 2022), pp. 1662-1671. DOI: 10.1038/s41386-022-01351-z. URL: https://doi.org/10.1038/s41386-022-01351-z.
[93] Qiang Yang et al. Transfer Learning. Cambridge University Press, 2020. DOI: 10.1017/9781139061773.
[94] Tal Yarkoni. “The generalizability crisis”. In: Behav Brain Sci (2020), pp. 1-37. ISSN: 0140-525X, 1469-1825. DOI: 10.1017/s0140525x20001685.
[95] Tal Yarkoni and Jacob Westfall. “Choosing Prediction Over Explanation in Psychology: Lessons From Machine Learning”. In: Perspect Psychol Sci 12.6 (2017), pp. 1100-1122. ISSN: 1745-6916, 1745-6924. DOI: 10.1177/1745691617693393.
[96] Nicole H. Yuen, Nathaniel Osachoff, and J. Jean Chen. “Intrinsic Frequencies of the Resting-State fMRI Signal: The Frequency Dependence of Functional Connectivity and the Effect of Mode Mixing”. In: Front. Neurosci. 13 (2019). ISSN: 1662-453X. DOI: 10.3389/fnins.2019.00900.
[97] David H. Zald et al. “Meta-Analytic Connectivity Modeling Reveals Differential Functional Connectivity of the Medial and Lateral Orbitofrontal Cortex”. In: Cereb. Cortex 24.1 (2012), pp. 232-248. ISSN: 1460-2199, 1047-3211. DOI: 10.1093/cercor/bhs308.
[99] Teresa Zawadzka et al. “Graph Representation Integrating Signals for Emotion Recognition and Analysis”. In: Sensors 21.12 (June 2021), p. 4035. DOI: 10.3390/s21124035. URL: https://doi.org/10.3390/s21124035.
[99] Ruibin Zhang et al. “Rumination network dysfunction in major depression: A brain connectome study”. In: Prog. Neuropsychopharmacol. Biol. Psychiatry 98 (March 2019 2020), p. 109819. ISSN: 0278-5846. DOI: 10.1016/j.pnpbp.2019.109819.
[100] Hong Zhu et al. “Nodal Memberships to Communities of Functional Brain Networks Reveal Functional Flexibility and Individualized Connectome”. In: Cereb. Cortex 31.11 (2021), pp. 5090-5106. ISSN: 1047-3211, 1460-2199. DOI: 10.1093/cercor/bhab144.
[101] Yitan Zhu et al. “Ensemble transfer learning for the prediction of anti-cancer drug response”. In: Scientific Reports 10.1 (October 2020). DOI: 10.1038/s41598-020-74921-0. URL: https://doi.org/10.1038/s41598-020-74921-0.

1. A method of connectome ensemble transfer learning, comprising:
selecting one or more target domains,
wherein the selected one or more target domains comprises one or more neuropsychological phenotypes of interest;
obtaining one or more modalities of target network connectivity data (TNCD) from one or more pluralities of individual subjects, wherein the one or more pluralities of individual subjects constitute target subjects;
selecting one or more source domains,
wherein the selected one or more source domains comprises one or more source phenotypes related to the one or more target neuropsychological phenotypes of interest;
obtaining one or more modalities of source network connectivity data (SNCD) from one or more pluralities of individual subjects, wherein the one or more pluralities of individual subjects constitute source subjects;
sampling, by a processing device, a plurality of source Connectome Graphical Models (sCGM) from the obtained SNCD for each source subject,
wherein the processing device comprises desktops or servers, further comprising central processors, graphics processors, tensor processors, or quantum processors; and
wherein said sampling comprises one or more subprocesses of connectome ensemble feature engineering, further comprising:
assigning two or more unique recipes of Network Connectivity Data Attributes (NCDA) to two or more respective sCGMs,
wherein each assigned unique recipe of NCDA constitutes a sCGM view; and
wherein the sampled one or more pluralities of sCGM views constitutes one or more source connectome ensemble representations (sCER);
pruning the one or more sampled sCER, whereby said pruning comprises:
eliminating, selecting, transforming, or otherwise reducing dimensionality of one or more sCGM views from the one or more sCER to produce one or more pruned sCERs;
transferring the one or more pruned sCER from the one or more source domains to the one or more target domains, said transfer further comprising:
extracting, by the processing device, a plurality of target CGM (tCGM) from the obtained TNCD for each target subject,
wherein said extraction comprises one or more subsequent subprocesses of connectome ensemble feature engineering, further comprising:
re-assigning the unique recipes of NCDA from each pruned sCER to one or more respective tCGMs,
wherein each re-assigned unique recipe of NCDA constitutes a tCGM view; and
wherein the extracted plurality of tCGM views constitutes one or more target connectome ensemble representations (tCER);
selecting one or more graph embedding algorithms;
embedding, by the processing device and the selected one more graph embedding algorithms, the transferred one or more tCER from each target subject, as one or more target Connectome Feature Vectors (tCFVs) for each target subject,
whereby said embedding projects the re-assigned NCDA of the extracted plurality of tCGM views into one or more lower-dimensional feature-spaces;
selecting one or more machine learning models in the target domain,
wherein the selected one or more machine learning models at least partially consumes the transferred and embedded one or more tCFVs from each target subject; and
wherein the selected one or more machine learning models constitutes a Connectome Ensemble Predictive Model (CEPM);
splitting the obtained one or more pluralities of target subjects into training, testing, and validation subsets;
training, by the processing device, the selected CEPM in the target domain, whereby said training comprises:
selecting a cost function,
wherein the selected cost function evaluates the performance of the selected CEPM; and
wherein the selected cost function is optimized during the training process;
selecting an optimization algorithm,
wherein the selected optimization algorithm updates the parameters of the selected CEPM to minimize the selected cost function;
feeding the training subset of the obtained one or more pluralities of target subjects into the selected CEPM,
wherein the training subset comprises the embedded tCFVs of each transferred tCGM view and the corresponding one or more target neuropsychological phenotypes of interest; and
whereby said feeding further comprises recursively partitioning the training subset;
initializing the selected CEPM; and
adjusting the parameters of the initialized CEPM based on the selected optimization algorithm and the selected cost function;
validating, by the processing device and for each tCGM view, the trained CEPM in the target domain, whereby said validating comprises:
feeding the validation subset of the obtained one or more pluralities of target subjects into the trained CEPM,
wherein the validation subset comprises the embedded tCFVs of each transferred tCGM view and the corresponding one or more target neuropsychological phenotypes of interest;
evaluating the performance of the trained CEPM on the validation subset using the selected cost function,
wherein a satisfactory performance indicates that training performance of the CEPM generalizes to unseen data;
adjusting, when applicable, the machine-learning hyperparameters of the selected CEPM, based on the performance on the validation subset,
wherein said adjusting comprises a grid search, random search, or Bayesian optimization of the machine-learning hyperparameters;
re-training, when applicable, the selected CEPM with the adjusted machine-learning hyperparameters; and
selecting the one or more tCGM views with the best performance on the validation subset, based on the selected cost function,
wherein the selected one or more tCGM views constitute the optimal tCER;
testing, by the processing device, the trained and validated CEPM in the target domain, whereby said testing comprises:
feeding the testing subset of the obtained one or more pluralities of target subjects into the trained and validated CEPM,
wherein the testing subset comprises the embedded tCFVs and the corresponding one or more target neuropsychological phenotypes of interest; and
evaluating the performance of the trained and validated CEPM on the testing subset using the selected cost function,
wherein a satisfactory performance indicates the effectiveness of the CEPM in predicting the one or more target neuropsychological phenotypes of interest;
storing, on one or more forms of non-transitory machine-readable storage media, the one or more embedded and trained outputs, wherein the output comprise: the weights of the one or more trained CEPM, the unique recipes of NCDA from the selected optimal tCER, and the selected one or more embedding algorithms; and
deploying the stored one or more stored outputs in the target domain to predict the one or more neuropsychological phenotypes of interest.

2. The method of claim 1, wherein the one or more target neuropsychological phenotypes of interest comprises one or more:
(i) diagnostic categories of neuropsychological disease,
(ii) prognostic trajectories of neuropsychological disease,
(iii) symptom severity in one or more diagnostic categories of neuropsychological disease,
(iv) neural, cognitive, behavioral, or emotional traits or states,
(v) neuropsychological treatment outcomes, response profiles, or side-effect profiles,
(vi) biomarkers of neuropsychological disease,
(vii) neurodevelopmental milestones,
(viii) neurodegenerative stages,
(ix) genetic or environmental risk factors for neuropsychological disease, or
(x) cognitive or emotional abilities.

3. The method of claim 1, wherein the one or more source phenotypes comprises one or more:
(i) neuropsychological disease or treatment mechanisms,
(ii) risk or protective factors for neuropsychological disease; or
(iii) genetic, epigenetic, neural, molecular, physiological, social, demographic, environmental, developmental, cognitive, emotional, or behavioral traits or states.

4. The method of claim 1, whereby said pruning by selection further comprises:
choosing one or more selection criteria, comprising:
(i) biological, functional, statistical, clinical, or practical significance,
(ii) explanation of variance, importance, influence, or predictability,
(iii) interpretability or explainability,
(iv) reliability, validity, or discriminability,
(v) covariance or mutual information, or
(vi) domain expertise or prior beliefs.
isolating one or more sCGM views, or its Network Connectivity Data Elements (NCDE), from the one or more sampled sCER, according to said chosen one or more elimination criteria.

5. The method of claim 1, whereby said pruning by elimination further comprises:
choosing one or more elimination criteria, comprising:
(i) biological, statistical, or theoretical implausibility,
(ii) domain unspecificity,
(iii) computational, financial, or practical infeasibility,
(iv) unreliability, invalidity, or indiscriminability,
(v) bias, unfairness, or inequity,
(vi) unpredictability or unimportance,
(vii) abnormality, invariance, uncertainty, sparsity, error, noise, or unrepresentativeness, or
(viii) uninterpretability or unexplainability.
removing one or more redundant, invariant, outlying, or artifactual sCGM, or its NCDE, according to said chosen one or more elimination criteria.

6. The method of claim 1, whereby said pruning by transformation comprises:
quantizing, normalizing, standardizing, or scaling the NCDE; or
fusing two or more sCGM views from the one or more sCER using one or more self-attention mechanisms, said fusion comprising:
learning a vector of attention weights for each view of the two or more sCGM views;
multiplying the learned attention weights by the NCDE of the corresponding two or more sCGM views;
encoding the weighted NCDE across the two or more sCGM views; and
computing a multi-head attention fused sCER of the two or more sCGM views from the aggregated weighted NCDE.

7. The method of claim 1, whereby said transferring alternatively comprises a method of decision-tree learning in the selected one or more source domains, said decision-tree learning further comprising:
selecting one or more graph embedding algorithms;
embedding, by the processing device and the selected one more graph embedding algorithms, the sCGM views of the one or more pruned sCER as one or more source Connectome Feature Vectors (sCFVs) for each source subject,
whereby said embedding projects the assigned NCDA of the sCGM views into one or more lower-dimensional feature-spaces;
selecting one or more tree-based learning algorithms such as decision trees, random forests, gradient-boosted trees, and extreme gradient-boosted trees;
initializing the selected one or more tree-based learning algorithms;
feeding the one or more embedded sCFVs from each source subject into the initialized one or more tree-based learning algorithms,
wherein the feeding comprises the embedded sCFVs and the corresponding one or more source phenotypes;
training the selected one or more tree-based learning algorithms on the fed one or more embedded sCFVs and the corresponding one or more source phenotypes; and
transferring the trained one or more tree-based learning algorithms to the selected one or more target domains, whereby said transfer further comprises:
applying the trained one or more tree-based learning algorithms to the obtained TNCD for each target subject to predict one or more optimized recipes of NCDA to for the one or more subsequent subprocesses of connectome ensemble feature engineering.

8. The method of claim 1, whereby said transferring alternatively comprises a method of co-training the CEPM with multiple tCGM views, said co-training further comprising:
selecting the embedded tCFVs corresponding to two or more tCGM views from the one or more tCER;
training, by the processing device, two or more CEPMs in the target domain, each CEPM corresponding to one of the selected two or more tCGM views;
wherein each CEPM is trained on the embedded tCFVs and the corresponding one or more target neuropsychological phenotypes of interest for its respective tCGM view;
iteratively and alternately refining the two or more trained CEPMs by training each CEPM on the most confidently predicted target subjects from the other CEPM,
wherein the confidence of the prediction is determined by the selected cost function or one or more additional cost functions;
combining the two or more refined CEPMs into a single co-trained CEPM, said combination comprising:
averaging, voting, stacking, or fusing the predictions of the two or more refined CEPMs for each target subject; and
evaluating the performance of the co-trained CEPM on the validation and testing subsets using the selected cost function.

9. The method of claim 1, whereby said transferring alternatively comprises a method of pretraining and fine-tuning one or more connectome ensemble transformer models (CETMs), said method further comprising:
embedding, by the processing device and the selected one more graph embedding algorithms, the sCGM views of the one or more pruned sCER as one or more source Connectome Feature Vectors (sCFVs) for each source subject,
whereby said embedding projects the assigned NCDA of the sCGM views into one or more lower-dimensional feature-spaces;
pretraining, by the processing device, one or more connectome ensemble transformer models (CETMs), said pretraining further comprising:
selecting one or more multi-view neural network architectures,
wherein the selected one or more architectures comprises one or more layers whose connections are defined by one or more sets of weights and bias;
selecting one or more unsupervised, semi-supervised, self-supervised, or reinforcement learning algorithms;
initializing the selected one or more multi-view neural network architectures;
feeding the one or more embedded sCFVs from each source subject into the initialized one or more multi-view neural network architectures;
training, by the selected one or more learning algorithms, the weights and biases of the initialized one or more multi-view neural network architectures,
wherein the training process aims to capture generalized patterns of graph topology of the sCER; and
wherein the trained one or more multi-view neural network architectures constitutes one or more CETMs;
storing, by the processing device and on one or more forms of non-transitory machine-readable storage media, the architecture, weights, and biases of the one or more pretrained CETMs;
fine-tuning, by the processing device and the splitting, training, validating, testing, storing, and deploying steps of claim 1, the one or more pretrained CETMs to the selected target domain, whereby said fine-tuning further comprises:
selecting one or more supervised learning algorithms;
initializing the one or more pretrained CETMs with the stored architecture, weights, and biases;
feeding the one or more embedded source Connectome Feature Vectors (sCFVs) and their corresponding target labels or annotations from the training dataset into the initialized one or more pretrained CETMs;
training the weights and biases of the initialized one or more pretrained CETMs using the selected one or more supervised learning algorithms, aiming to minimize a task-specific loss function;
wherein the training process focuses on adapting the pretrained CETMs to the specific target domain or task;
evaluating the performance of the fine-tuned one or more CETMs on a separate validation or test dataset, ensuring that they generalize well to new, unseen data; and
storing the architecture, weights, and biases of the fine-tuned one or more CETMs as one or more Connectome Ensemble Predictive Models (CEPMs) on one or more forms of non-transitory machine-readable storage media.

10. The method of claim 1, wherein said pruning is achieved through one or more intermediate analyses, further comprising:
(i) Bayesian analysis,
(ii) auxiliary or multi-task learning,
(iii) Multiverse Analysis,
(iv) Sensitivity Analysis,
(v) paired model comparisons,
(vi) Structural Equation Modeling, or
(vii) Factor Analysis.

11. The method of claim 1, whereby said embedding of the transferred one or more tCER into one or more respective tCFVs further comprises:
applying one or more global or local graph theory algorithms, matrix factorization algorithms, spectral clustering algorithms, manifold learning algorithms, encoders, decoders, or autoencoders to the NCDE of the one or more tCGM views of the one or more tCERs; or
applying one or more omnibus, multi-view, or multi-aspect graph embedding algorithms, hypergraph embedding algorithms, or multi-layer graph embedding algorithms to the NCDE of the complete set, a subset, or plurality of subsets of the one or more tCGM views of the one or more tCERs.

12. The method of claim 1, wherein the one or more tCER or its embedded tCFV is represented as one or more respective multigraphical or hypergraphical models,
wherein the one or more transferred multigraphical models comprises a plurality of multiplicatively attributed tCGM views that form distinct graphical layers, the nodes of which are aligned or matched;
wherein the one or more transferred hypergraphical models encodes one or more pairwise similarity relationships between one or more pairs of tCFV constituents of the one or more tCER; and
wherein the CEPM are trained on the one or more multigraphical or hypergraphical models.

13. The method of claim 1, wherein said transferring further comprises:
evaluating the quality of domain adaptation and alignment; and
refining the transfer process by adjusting the one or more source or target CERs, the pruning process, the embedding algorithms, the CEPM, or the cost function, based on the evaluated quality of domain adaptation and alignment.

14. The method of claim 1, whereby the stored outputs are provided to a user interface or user experience that allows the user to interact with the CEPM, monitor the training, and visualize the results of the evaluation.

15. A method of multimodal connectome ensemble domain adaptation, said method comprising:
selecting one or more target domains including one or more target neuropsychological phenotypes of interest;
obtaining source network connectivity data (SNCD) from one or more pluralities of individual subjects in one or more source domains;
selecting one or more target modalities,
wherein the one more target modalities of TNCD tracks variance in the selected one or more target neuropsychological phenotypes of interest;
wherein the one more target modalities of TNCD tracks variance in the obtained SNCD; and
wherein the one more modalities of TNCD encodes two or more Network Connectivity Data Attributes (NCDA);
obtaining target network connectivity data (TNCD) from the selected one or more target modalities for the one or more pluralities of individual subjects in one or more target domains;
sampling, by a processing device, a plurality of source Connectome Graphical Models (sCGM) from the obtained SNCD for each source subject,
wherein the processing device comprises desktops or servers, further comprising central processors, graphics processors, tensor processors, or quantum processors; and
wherein said sampling comprises one or more subprocesses of connectome ensemble feature engineering, further comprising:
assigning two or more unique recipes of NCDA to two or more respective sCGMs,
wherein each assigned unique recipe of NCDA constitutes a sCGM view; and
wherein the sampled one or more pluralities of sCGM views constitutes one or more source connectome ensemble representations (sCER);
sampling, by a processing device, a plurality of target Connectome Graphical Models (tCGM) from the obtained TNCD for each target subject,
wherein the processing device comprises desktops or servers, further comprising central processors, graphics processors, tensor processors, or quantum processors; and
wherein said sampling comprises one or more subprocesses of connectome ensemble feature engineering, further comprising:
assigning two or more unique recipes of NCDA to two or more respective tCGMs,
wherein each assigned unique recipe of NCDA constitutes a tCGM view; and
wherein the sampled one or more pluralities of tCGM views constitutes one or more target connectome ensemble representations (tCER);
aligning or fusing, by the processing device and through an iterative process of recursive partitioning, the sampled one or more sCER with the sampled one or more tCER, wherein said alignment or fusion further comprises:
selecting one or more sCGM views from the one or more sCER;
selecting one or more tCGM views from the one or more tCER;
selecting one or more multi-view representational alignment or fusion algorithms,
wherein said alignment isolates one or more shared latent representations between the selected one or more sCGM views from the sampled one or more sCER, and the selected one or more tCGM views from the sampled one or more tCER; and
wherein said fusion distills the selected one or more sCGM views from the sampled one or more sCER, and the selected one or more tCGM views from the sampled one or more tCER into one or more compressed latent representations;
selecting one or more alignment or fusion optimization objectives,
wherein the selected one or more alignment or fusion optimization objectives minimize latent distance between the NCDE of the selected one or more sCGM and the NCDE of the selected one or more tCGM;
learning one or more mapping kernels that project the NCDE of the selected one or more sCGM views, and the NCDE of the selected one or more tCGM views, onto one or more shared or fused latent spaces, respectively,
wherein the learned one or more mapping kernels optimize the selected one or more alignment or fusion optimization objectives;
jointly embedding, by the learned one or more mapping kernels, the one or more sCGM and tCGM into one or more multimodal connectome feature vectors (mCFV);
selecting one or more alignment or fusion evaluation metrics that gauge the selected one or more alignment optimization objectives;
evaluating, using the selected one or more alignment or fusion evaluation metrics, the quality of the alignment or fusion between the sampled one or more sCER and the sampled one or more tCER;
adjusting, by the processing device, the one or more mapping kernels based on the selected alignment or fusion optimization objectives and the evaluation of the alignment or fusion quality; and
obtaining a final jointly embedded one or more mCFVs after convergence of the iterative alignment process;
selecting one or more machine learning models in the target domain,
wherein the selected one or more machine learning models at least partially consumes the transferred and embedded one or more tCFVs from each target subject; and
wherein the selected one or more machine learning models constitutes a Multimodal Connectome Ensemble Predictive Model (mCEPM);
splitting the obtained one or more pluralities of target subjects into training, testing, and validation subsets;
training, by the processing device, the selected one or more mCEPMs using the final jointly embedded one or more mCFVs, whereby said training comprises:
selecting a cost function,
wherein the selected cost function evaluates the performance of the selected one or more mCEPM; and
wherein the selected cost function is optimized during the training process;
selecting an optimization algorithm,
wherein the selected optimization algorithm updates the parameters of the selected one or more mCEPM to minimize the selected cost function;
feeding the training subset of the obtained one or more pluralities of target subjects into the selected one or more mCEPM,
wherein the training subset comprises the final jointly embedded one or more mCFVs of each transferred tCGM view and the corresponding one or more target neuropsychological phenotypes of interest; and
whereby said feeding further comprises recursively partitioning the training subset;
initializing the trained one or more mCEPM; and
adjusting the parameters of the initialized mCEPM based on the selected optimization algorithm and the selected cost function;
validating, by the processing device and for each tCGM view, the trained one or more mCEPM in the target domain, whereby said validating comprises:
feeding the validation subset of the obtained one or more pluralities of target subjects into the trained one or more mCEPM,
wherein the validation subset comprises the final jointly embedded one or more mCFVs of each transferred tCGM view and the corresponding one or more target neuropsychological phenotypes of interest;
evaluating the performance of the trained one or more mCEPM on the validation subset using the selected cost function,
wherein a satisfactory performance indicates that training performance of the one or more trained mCEPM generalizes to unseen data;
adjusting, when applicable, the machine-learning hyperparameters of the trained one or more mCEPM, based on the performance on the validation subset,
wherein said adjusting comprises a grid search, random search, or Bayesian optimization of the machine-learning hyperparameters;
re-training, when applicable, the trained one or more mCEPM with the adjusted machine-learning hyperparameters; and
selecting the one or more tCGM views with the best performance on the validation subset, based on the selected cost function,
wherein the selected one or more tCGM views constitute the optimal tCER;
testing, by the processing device, the trained and validated one or more mCEPM in the target domain, whereby said testing comprises:
feeding the testing subset of the obtained one or more pluralities of target subjects into the trained and validated one or more mCEPM,
wherein the testing subset comprises the final jointly embedded one or more mCFVs and the corresponding one or more target neuropsychological phenotypes of interest; and
evaluating the performance of the trained and validated one or more mCEPM on the testing subset using the selected cost function,
wherein a satisfactory performance indicates the effectiveness of the trained and validated one or more mCEPM in predicting the one or more target neuropsychological phenotypes of interest;
storing, on one or more forms of non-transitory machine-readable storage media, the one or more embedded and trained outputs, wherein the output comprise: the weights of the trained and validated one or more mCEPM, the unique recipes of NCDA from the selected optimal tCER, and the selected one or more embedding algorithms; and
deploying the stored one or more stored outputs in the target domain to predict the one or more neuropsychological phenotypes of interest.

16. The method of claim 15, wherein the selected one or more alignment or fusion optimization objectives comprises:
(i) minimization of domain discrepancy,
(ii) relevance to the one or more target neuropsychological phenotypes,
(iii) biological or functional significance,
(iv) statistical significance,
(v) information content,
(vi) contribution to explained variance, or
(vii) compatibility with the one or more statistical learning models.

17. The method of claim 15, wherein the selected one or more alignment algorithms comprises:
(i) Distance or similarity-based alignment such as Cross-Modal Ranking, Partial Least Squares, Cross-Modal Hashing, and Deep Cross-View Embedding Models; or
(ii) Correlation-based alignment such as Canonical Correlation Models, Sparse CCA, Kernel CCA, and deep CCA.

18. The method of claim 15, wherein the selected one or more fusion algorithms comprises:
(i) graphical fusion such as multi-modal topic learning, multi-view sparse coding, multi-view latent space Markov networks, and multi-modal deep Boltzmann machines; or
(ii) neural network fusion such as multi-modal autoencoders, multi-view convolutional neural networks, multi-layer graph neural networks, hypergraph neural networks, Domain Adversarial Neural Networks, and multi-modal recurrent neural networks.

19. The method of claim 15, whereby the user initializes, by the processing device, the one or more stored training outputs to opportunistically predict the one or more target neuropsychological phenotypes of interest,
whereby removing the SNCD from the aligned one or more mCFVs preserves the predictive performance of the trained one or more CEPMs, when relying only on the TNCD; and
whereby the one or more target neuropsychological phenotypes of interest are predicted using only the obtained TNCD and the one or more aligned mCFVs.

20. The method of claim 15, wherein a first embodiment of the one or more modalities of SNCD comprises:
(i) structural, functional, or effective brain connectivity data extracted from one or more neuroimaging data samples, or
(ii) single nucleotide polymorphism (SNP) genotype, gene co-expression profiles, Gene Regulatory Networks (GRN), or DNA methylation connectivity data extracted from one or more genomic data samples.

21. The method of claim 15, wherein a first embodiment of the one or more modalities of TNCD comprises behavioral, molecular, social, or portable neuroimaging data modalities:
(i) digital connectivity data obtained from physical movement patterns, social interactions, or usage patterns of electronic devices data samples,
(ii) speech, language, knowledge, or text connectivity data obtained from interviews, questionnaires, or natural language processing analysis of written or verbal communication data samples,
(iii) physiological connectivity data obtained from wearable sensors, such as heart rate, galvanic skin conductance, or body temperature monitoring data samples;
(iv) metabolomic or proteomic profiles obtained from blood, cerebrospinal fluid, or other molecular concentration array data samples;
(v) social network connectivity data obtained from online platforms or offline interactions data samples; or
(vi) portable neuroimaging connectivity data obtained from Electroencephalography (EEG), Magnetoencephalography (MEG), Functional Near-Infrared Spectroscopy (fNIRS), or Application-Specific Integrated Circuits (ASIC).

22. The method of claim 15, wherein transferring CER further comprises:
transferring CER across one or more pairs of individual persons, wherein the tCER constitutes the CER of the other individual person in the one or more pairs of individual persons;
aligning and jointly embedding the sCER and the tCER, wherein the alignment and joint embedding constitute the alignment of phenotypes across the one or more pairs of individual persons; and
predicting the one or more target neuropsychological phenotypes of interest in the target domain using the aligned phenotypes across the one or more pairs of individual persons with the multimodal connectome ensemble transfer learning method.

23. The method of claim 15, wherein transferring CER further comprises:
transferring CER across one or more pairs, including an individual person and artificially intelligent agent, wherein the tCER constitutes the CER of the artificially intelligent agent;
aligning and jointly embedding the sCER and the tCER, wherein the alignment and joint embedding constitute the alignment of phenotypes across the one or more pairs; and
aligning one or more neuropsychological phenotypes from the individual person with the artificially intelligent agent in the one or more pairs using the multimodal connectome ensemble transfer learning method.

24. The method of claim 15, wherein transferring CER further comprises:
transferring CER across one or more pairs, including an individual person and artificially intelligent agent, wherein the tCER constitutes the CER of the individual person;
aligning and jointly embedding the sCER and the tCER, wherein the alignment and joint embedding constitute the alignment of phenotypes across the one or more pairs; and
aligning one or more neuropsychological phenotypes from the artificially intelligent agent with the individual person in the one or more pairs using the multimodal connectome ensemble transfer learning method.

25. A system of synchronized computer hardware that implements connectome ensemble transfer learning, said system comprising:
one or more processing devices,
wherein the one or more processing devices comprise central processors, graphics processors, tensor processors, or quantum processors;
wherein the one or more processing devices comprise one or more forms of random access memory and cache storage;
wherein the one or more processing devices support the various methods of automated feature engineering of connectome ensembles, pruning of connectome ensemble representations, embedding of connectome ensemble representations, multimodal connectome ensemble fusion, domain adaptation and alignment, training of connectome ensemble transfer learning machine models, and deployment of the trained models for clinical use; and
wherein the one or more processing devices comprise a distributed or parallel computing architecture that initializes and executes one or more Directed Acyclic Graphs, facilitating efficient processing of unimodal or multimodal multiplicatively attributed network connectivity data;
one or more non-transitory machine-readable storage media for storing the obtained target and source network connectivity data, the sampled Connectome Graphical Models (CGMs), the connectome ensemble representations (CERs), the Connectome Feature Vectors (CFVs), the trained Connectome Ensemble Predictive Models (CEPMs), and their associated parameters;
one or more communication interfaces for facilitating the transfer of data and information between the one or more processing devices and the storage media,
wherein the one or more communication interfaces comprise one or more gateways configured to receive and process data from a wide variety of network connectivity data modalities, data formats, and hardware configurations;
one or more input devices for enabling users to interact with the system and provide input for selecting target and source domains, obtaining and preprocessing network connectivity data, and monitoring CEPM training;
one or more output devices for displaying the results of the evaluations, visualizations, and predictions generated by the CEPM, enabling users to make informed decisions about precision mental healthcare interventions;
one or more user interfaces for providing a user-friendly environment to access and interact with the system, input data, configure settings, and view the results; and
an operating system configured to manage the system resources, execute the processes, and coordinate the activities of the one or more processing devices, communication interfaces, input devices, output devices, and user interfaces,
wherein the one or more operating systems are configured to adapt its processes and workflows dynamically based on available computational resources, data quality, and user-defined constraints or requirements.

26. The system of claim 25, wherein an embodiment further comprises software applications or tools that enable users to monitor the performance of the CEPMs, visualize the results, and generate reports or summaries of the evaluations and predictions for use in clinical decision-making or research.

27. The system of claim 25, wherein an embodiment further comprises security and privacy mechanisms to protect the confidentiality, integrity, and availability of the data, models, and results, including encryption, access controls, and data privacy-preserving protocols.

28. The system of claim 25, wherein the one or more communication interfaces are connected to or integrated with one or one or more electronic health record (EHR) systems, clinical decision support systems (CDSS), or health information exchange (HIE) platforms to facilitate data exchange, interoperability, or real-time deployment of the CEPMs in clinics.

29. The system of claim 25, wherein an embodiment of the system of synchronized computer hardware comprises one or more cloud-based services that provide access to the connectome ensemble transfer learning methods and CEPMs through web-based interfaces, APIs, or other remote access methods.

30. The system of claim 25, wherein an embodiment of the one or more output devices comprises one or more configurations that provides real-time, personalized predictions for precision mental healthcare interventions based on the CEPMs, supporting the delivery of high-precision risk scores, diagnoses, prognoses, and treatments for individual patients or populations.
